(19) United States
(12) Patent Application Publication
(10) Pub. No.: US 2017/0145381 A1
(43) Pub. Date: May 25, 2017

(54) METHOD OF GENERATING MULTILINEAGE POTENTIAL CELLS FROM LYMPHOCYTES

(71) Applicant: Fuwan Pty Ltd, Eight Mile Plains (AU)

(72) Inventors: Shou-hsiung PAI, Eight Mile Plains (AU); Yi-jen LEE, Eight Mile Plains (AU); Jah-yao LIU, Eight MIle Plaind (AU)

(21) Appl. No.: 15/316,473

(22) PCT Filed: Jun. 4, 2015
(86) PCT No.: PCT/AU2015/050306

§ 371 Date: Dec. 5, 2016

(30) Foreign Application Priority Data
Jun. 6, 2014 (AU) 2014902175

(51) Int. Cl. C12N 5/074; G01N 33/50; A61K 35/545

(52) U.S. Cl. CPC C12N 5/0607; A61K 35/545; G01N 33/5073; G01N 33/502; C12N 2506/11

(57) ABSTRACT

The present invention relates generally to a method of generating cells exhibiting multilineage potential and to cells generated thereby. More particularly, the present invention is directed to an in vitro method of generating mammalian stem cells from CD4* mononuclear cells, CD8* mononuclear cells, CD25* mononuclear cells, CD19* mononuclear cells or CD20* mononuclear cells and to cells generated thereby. This finding has now facilitated the design of means for reliably and efficiently generating populations of multilineage potential cells, such as stem cells, for use in a wide variety of clinical and research settings. These uses include, inter alia, the directed differentiation, either in vitro or in vivo, of the subject multilineage potential cells and the therapeutic or prophylactic treatment of a range of conditions either via the administration of the multilineage potential cells of the invention or the more fully differentiated cellular populations derived therefrom. Also facilitated is the design of in vitro based screening systems for testing the therapeutic impact and/or toxicity of potential treatment or culture regimes to which these cells may be exposed.

### FIELD OF THE INVENTION

The present invention relates generally to a method of generating cells exhibiting multilineage potential and to cells generated thereby. More particularly, the present invention is directed to an in vitro method of generating mammalian stem cells from CD4⁺ mononuclear cells, CD8⁺ mononuclear cells, CD25⁺ mononuclear cells, CD19⁺ mononuclear cells or CD20⁺ mononuclear cells and to cells generated thereby. This finding has now facilitated the design of means for reliably and efficiently generating populations of multilineage potential cells, such as stem cells, for use in a wide variety of clinical and research settings. These uses include, inter alia, the directed differentiation, either in vitro or in vivo, of the subject multilineage potential cells and the therapeutic or prophylactic treatment of a range of conditions either via the administration of the multilineage potential cells of the invention or the more fully differentiated cellular populations derived therefrom. Also facilitated is the design of in vitro based screening systems for testing the therapeutic impact and/or toxicity of potential treatment or culture regimes to which these cells may be exposed.

### BACKGROUND OF THE INVENTION

Bibliographic details of the publications referred to by author in this specification are collected alphabetically at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

There is considerable interest in the identification, isolation and generation of mammalian stem and progenitor cells. Reference to “stem cells” and “progenitor cells” is generally understood to encompass a wide variety of cell types including both totipotent cells which can generate any cell type (including germ cells) and pluripotent precursor cells which are capable of generating a more limited variety of mature cell lineages. Some precursor cell types are still more differentiated and correspond to precursors capable of generating cells of specific cell lineages. These abilities serve as the basis for all the cellular differentiation and specialisation necessary for complete organ and tissue development.

In terms of reproducing, in vitro, selected aspects of this developmental pathway, there has been much focus on the isolation and culturing of stem cells. Embryonic stem cells, for example, can be established by culturing the blastocyst inner cell mass derived cells and frequently repeating dissociation and subculturing. Under appropriate conditions, in vitro culturing can be maintained while maintaining both the normal karyotype and the totipotency of the stem cells. Significant progress has also been made in terms of facilitating the differentiation of stem cells along a particular lineage. Although ES cells have been isolated from humans, their use in research and therapy is hampered by ethical considerations.

Adult tissues also contain populations of stem cells that can self-replicate and give rise to daughter cells that undergo an irreversible terminal differentiation (Science, 287, 1442-1446, 2000). The best-characterized are hematopoietic stem cells and their progeny, but stem cells are identified in most of the tissues, including mesenchymal, neuron, and hemotopoietic cells (Science, 284, 143-147, 1999; Science, 287, 1433-1438, 2000; J. Hepatol., 29, 676-682, 1998). Mesenchymal stem cells are identified as adherent fibroblast-like cells in the bone marrow with differentiation potential into mesenchymal tissues, including bone, cartilage, fat, muscle, and bone marrow stroma (Science, 284, 143-147, 1999). Mesenchymal progenitors having morphologic and phenotypic features and differentiation potentials similar to mesenchymal stem cells and have been reported at extremely low frequencies in umbilical cord blood (Br. J. Haematol., 109, 235-242, 2000), fetal (Blood, 98, 2396-2402, 2001) and adult peripheral blood (Arthritis Res., 2, 477-488, 2000).

To this end, differentiation has always been assumed to take the form of a linear progression of the stem cell through the regulation of many genes to ultimately attain the phenotype of a terminally differentiated somatic cell, whose function is clearly defined and whose lifespan is limited. Examples of such cells include red blood cells, osteoclasts, islet cells and platelets. The stem cell is thought to divide, renew itself and produce daughter cells for commitment to a specific somatic lineage (asymmetrical division). It is also thought that under appropriate environmental conditions, the stem cell can divide symmetrically to produce the doubling of the stem cell pool.

Nevertheless, the fact remains that the efficient and reliable isolation, maintenance and, particularly, expansion of stem cells continues to be elusive. Accordingly, there remains an ongoing need to develop new means for efficiently and reproducibly facilitating the isolation, maintenance and differentiation of stem cells.

In work leading up to the present invention, it has been determined that stem cell expansion does not necessarily need to occur by virtue of asymmetric stem cell division to provide both stem cell renewal and linear differentiation of the relevant daughter cell along a specific lineage through to terminal differentiation. Rather, expansion can be achieved by virtue of the transition of a mature cell back to a cell with multilineage potential. This finding has now facilitated the development of means for reliably and efficiently generating cells which exhibit multilineage potential, thereby providing a valuable mechanism by which stem cell populations and/or somatic cells differentiated therefrom can be made available for clinical and research use.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

One aspect of the present invention is directed to a method of generating mammalian multilineage potential cells, said method comprising establishing an in vitro cell culture which proportionally comprises:
(i) 10-40% v/v, or functionally equivalent proportion thereof, of a mononuclear cell suspension, which mononuclear cells express CD4, CD8, CD25, CD19 or CD20;
(ii) 5-40% v/v, or functionally equivalent proportion thereof, of an approximately 5%-85% albumin solution; and
(iii) 30-80% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In another aspect there is provided a method of generating mammalian multilineage potential cells, said method comprising establishing an in vitro cell culture which proportionally comprises:
(i) 10-40% v/v, or functionally equivalent proportion thereof, of a lymphocyte suspension, which lymphocytes express CD4, CD8, CD25, CD19 or CD20;
(ii) 5-40% v/v, or functionally equivalent proportion thereof, of an approximately 5%-85% albumin solution; and
(iii) 30-80% v/v, or functionally equivalent proportion thereof, of a cell culture medium
   wherein said cell culture is maintained for a time and under conditions sufficient to induce the transition of said monocytes cells to a cell exhibiting multilineage differentiative potential.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In still another aspect there is provided a method of generating mammalian multilineage potential cells, said method comprising establishing an in vitro cell culture which proportionally comprises:
(i) 10-40% v/v, or functionally equivalent proportion thereof, of a peripheral blood derived monocyte suspension, which mononuclear cells express CD4, CD8, CD25, CD19 or CD20;
(ii) 5-40% v/v, or functionally equivalent proportion thereof, of an approximately 5%-85% albumin solution; and
(iii) 30-80% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In yet another aspect there is provided a method of generating mammalian multilineage potential cells, said method comprising establishing an in vitro cell culture which proportionally comprises:
(i) 10-40% v/v, or functionally equivalent proportion thereof, of a mononuclear cell suspension, which mononuclear cells express CD4, CD8, CD25, CD19 or CD20;
(ii) 5-40% v/v, or functionally equivalent proportion thereof, of an approximately 5%-85% albumin solution; and
(iii) 30-80% v/v, or functionally equivalent proportion thereof, of a cell culture medium
   wherein said cell culture is maintained for a time and under conditions sufficient to induce the transition of said mononuclear cells to a cell exhibiting multilineage differentiative potential, which multilineage potential cell exhibits haematopoietic and/or mesenchymal potential.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In yet still another aspect there is provided a method of generating mammalian multilineage potential cells, said method comprising establishing an in vitro cell culture which proportionally comprises:
(i) 10-40% v/v, or functionally equivalent proportion therefore of a mononuclear cell suspension, which mononuclear cells express CD4, CD8, CD25, CD19 or CD20;
(ii) 5-40% v/v, or functionally equivalent proportion thereof, of an approximately 5%-20% albumin solution; and
(iii) 30-80% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In a further aspect there is provided a method of generating human multilineage potential cells, said method comprising establishing an in vitro cell culture which proportionally comprises:
(i) 10-40% v/v, or functionally equivalent proportion thereof, of a human peripheral blood mononuclear cell suspension, which mononuclear cells express CD4, CD8, CD25, CD19 or CD20;
(ii) 5-40% v/v, or functionally equivalent proportion thereof, of an approximately 5%-85% albumin solution; and
(iii) 30-80% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In another further aspect of the present invention there is provided a method of facilitating the generation of a mammalian MLPC-derived cell, said method comprising:

(i) establishing an in vitro cell culture which proportionally comprises:
(a) 10-40% v/v, or functionally equivalent proportion thereof, of a mononuclear cell suspension, which mononuclear cells express CD4, CD8, CD25, CD19 or CD20;
(b) 5-40% v/v, or functionally equivalent proportion thereof, of an approximately 5%-85% albumin solution; and
(c) 30-80% v/v, or functionally equivalent proportion thereof, of a cell culture medium
   wherein said cell culture is maintained for a time and under conditions sufficient to induce the transition of said mononuclear cells to a MLPC; and optionally
   (ii) contacting the MLPC of step (i) with a stimulus to direct the differentiation of said MLPC to a MLPC-derived phenotype.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In still another further aspect there is provided a method of facilitating the generation of a mammalian MLPC-derived cell, said method comprising:

(i) establishing an in vitro cell culture which proportionally comprises
(a) 10-40% v/v, or functionally equivalent proportion thereof, of a mononuclear cell suspension, which mononuclear cells express CD4, CD8, CD25, CD19 and CD20;
(b) 5-40% v/v, or functionally equivalent proportion thereof, of an approximately 5%-85% albumin solution; and
(c) 30-80% v/v, or functionally equivalent proportion thereof, of a cell culture medium
   wherein said cell culture is maintained for a time and under conditions sufficient to induce the transition of said mononuclear cells to a MLPC; and optionally
   (ii) contacting the MLPC step (i) with a stimulus to direct the differentiation of said MLPC to a haematopoietic or mesenchymal phenotype.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

Another aspect of the present invention is directed to a method of therapeutically and/or prophylactically treating a condition in a mammal, said method comprising administering to said mammal an effective number of MLPCs or partially or fully differentiated MLPC-derived cells which have been generated according to the method of the present invention.

In still another aspect there is provided a method of therapeutically and/or prophylactically treating a condition characterised by aberrant haematopoietic or mesenchymal functioning in a mammal, said method comprising administering to said mammal;
(i) an effective number of haematopoietic stem cells or partially or fully differentiated haematopoietic stem cell-derived cells which have been generated according to the method of the present invention; or
(ii) an effective number of mesenchymal stem cells or partially or fully differentiated mesenchymal stem cell-derived cells which have been generated according to the method of the present invention.

Another aspect of the present invention is directed to the use of a population of MLPCs or MLPC-derived cells, which cells have been generated in accordance with the method of the present invention, in the manufacture of a medicament for the treatment of a condition in a mammal.

Yet another aspect of the present invention is directed to MLPCs or MLPC-derived cells and which have been generated in accordance with the method of the present invention.

### BRIEF DESCRIPTION OF THE DRAWINGS

FIG. 1 is a photographic representation of the morphology of CD4⁺ PBMCs at Day 1 (A) and Day 4 (B) post culture.

FIG. 2 is a photographic representation of the morphology of CD8⁺ PBMCs at Day 1 (A) and Day 4 (B) post culture.

FIG. 3 is a photographic representation of the morphology of CD19⁺ PBMCs Day 1 (A) and Day 4 (B) post culture.

FIG. 4 is a photographic representation of the morphology of CD25⁺ PBMCs Day 1 (A) and Day 4 (B) post culture.

FIG. 5 is a photographic representation of the morphology of CD20⁺ PBMCs at Day 1 (A) and Day 4 (B) post culture.

FIG. 6 is a photographical representation of the protein expression of (A) Nestin, GATA binding factor-4 (GATA-4) and Granulocyte-colony stimulating factor (G-CSF) (B) Caveolin in CD4⁺, CD8⁺, CD19⁺, CD20⁺ and CD25⁺ lymphocytes.

FIG. 7 is a photographical representation of the protein expression of (A) Actin (control), Synaptophysin (SYP) and Neurogenin 3 (B) β Enolase (ENO-3), Granzyme B (GZMB) and Nerve growth factor (NGF) in CD4⁺, CD8⁺, CD19⁺, CD20⁺ and CD25⁺ lymphocytes.

### DETAILED DESCRIPTION OF THE INVENTION

The present invention is predicated, in part, on the determination that adult stem cell expansion is not necessarily based on the occurrence of asymmetrical stem cell division in order to effect both stem cell renewal and differentiation along a specific somatic cell lineage. In particular, multipotent stem cells can be sourced from T lymphocytes which are induced to transition to a state of multilineage potential, this being followed by symmetrical division and differentiation under the appropriate stimulus. This finding is of significant importance since it has been a particular difficulty in the art that methods of efficiently inducing stem cell renewal and expansion in vitro have not been realised. The present invention therefore provides a means for the routine in vitro generation of mammalian stem cells based on inducing the de-differentiation of a mature mammalian cell to a stem cell phenotype which exhibits multilineage potential. Accordingly, the potential in vivo and in vitro applications of these findings are extremely widespread including, but not limited to, the in vitro generation of stem cell populations, directed differentiation of the subject stem cells either in vitro or in vivo, therapeutic or prophylactic treatment regimes based thereon and the in vitro assessment of the effectiveness and/or toxicity of potential treatment or culture regimes to which the cells of the invention may be exposed.

Accordingly, one aspect of the present invention is directed to a method of generating mammalian multilineage potential cells, said method comprising establishing an in vitro cell culture which proportionally comprises:
(i) 10-40% v/v, or functionally equivalent proportion thereof, of a mononuclear cell suspension, which mononuclear cells express CD4, CD8, CD25, CD19 or CD20;
(ii) 5-40% v/v, or functionally equivalent proportion thereof, of an approximately 5%-85% albumin solution; and
(iii) 30-80% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In another embodiment, said mononuclear cell suspension is 15% v/v or functionally equivalent proportion thereof, said 5-85% albumin solution is used at 15% v/v or functionally equivalent proportion thereof and said culture medium is 70% v/v or functionally equivalent proportion thereof.

Reference to a “mononuclear cell” should be understood as a reference to a cell with a single nucleus. In the context of leukocytes, this primarily describes monocytes and lymphocytes. The present invention is directed to the determination that mononuclear cells which express CD4, CD8, CD25, CD19 or CD20 can be induced to transition to a state of multilineage potential when cultured in accordance with the method of the present invention. Reference to a cell which expresses CD4, CD8, CD25, CD19 or CD20 should be understood as a reference to a mononuclear cell which expresses either or both of the CD4 and CD8 antigens or which expresses CD25 or CD19 or CD20. The expression of these cell surface molecules may be transient, such as the double-positive expression of CD4 and CD8 on thymocytes during T cell differentiation, or ongoing. However, it should be understood that irrespective of whether CD4/CD8 expression is transient or ongoing, the method of the present invention is directed to the use of cells which, at the time of initial culture, are expressing CD4 and/or CD8. A corresponding meaning should be understood to apply to cells expressing CD25 or CD19 or CD20. That is, it is a reference to a mononuclear cell which express CD25 or CD19 or CD20 either transiently or on an ongoing basis, provided that at the time of initial culture these cells are expressing one of these cell surface markers.

Without limiting the present invention to any one theory or mode of action, CD4 is a glycoprotein found on the surface of T helper cells, monocytes, macrophages and dendritic cells. It is a member of the immunoglobulin superfamily and comprises four immunoglobulin domains, D₁ to D₄. CD4 also has alternatively been known as leu-3 and T4. CD8 is predominantly expressed on the surface of cytotoxic T cells but can also be found on natural killer cells, natural killer T cells, cortical thymocytes and dendritic cells. CD8 takes the form of a dimer consisting of a pair of CD8 chains, most commonly a CD8-α and a CD8-β chain. Both these chains are also members of the immunoglobulin super family Although CD8 is most commonly expresses as a heterodimer, homodimers are also expressed on some cells, such as CD8-α homodimes. CD25 is the alpha chain of the IL-2 receptor. It is a type I transmembrane protein present on activated T cells, activated B cells, some thymocytes, myeloid precursors, and oligodendrocytes that associate with CD122 to form a heterodimer that can act as a high-affinity receptor for IL-2. Although CD25 has been used as a marker to identify regulatory T cells, it has been found that a proportion of resting memory T cells constitutively express CD25 in humans. The CD19 gene encodes a cell surface molecule that assembles with the antigen receptor of B lymphocytes in order to decrease the threshold for antigen receptor-dependent stimulation. It is expressed on follicular dendritic cells and B cells. In fact, it is present on B cells from the earliest recognizable B-lineage cells during development to B-cell blasts. However, it is lost on maturation to plasma cells. It primarily acts as a B cell co-receptor in conjunction with CD21 and CD81. Upon activation, the cytoplasmic tail of CD19 becomes phosphorylated, which leads to binding by Src-family kinases and recruitment of PK-3 kinase. Without limiting the present invention to any one theory or mode of action, CD20 is an activated-glycosylated phosphoprotein expressed on the surface of all B-cells beginning at the pro-B phase and progressively increasing in concentration until maturity.

Accordingly, in one embodiment, said CD4⁺ and/or CD8⁺ mononuclear cell is a thymocyte, T cell, natural killer cell, natural killer T cell, macrophage or dendritic cell.

In another embodiment, said CD25⁺ cell is a regulatory T cell or a memory T cell.

In still another embodiment, said CD19⁺ cell is a B cell of any stage of differentiation.

To this end, reference to “CD4”, “CD8”, “CD25”, “CD19” and “CD20” should be understood as a reference to all forms of CD4, CD8, CD25, CD 19 and CD20 and to functional mutant or polymorphic forms of these molecules, including isomeric forms which may arise from alternative splicing of the mRNA of these molecules. Reference to “CD4”, “CD8”, “CD25”, “CD19” and “CD20” should also be understood to include reference to all forms of these molecules including all precursor, proprotein or intermediate forms which may be expressed on the cell surface. It should also be understood to extend to any CD4, CD8, CD25, CD19 or CD20 cell surface molecule, whether existing as a dimer, multimer or fusion protein.

As detailed herein the CD4, CD8, CD25, CD19 and CD20 molecules are predominantly expressed extensively on lymphocytes and NK cells. Reference to “lymphocyte” should be understood as a reference to any lymphocyte or NK cell, irrespective of its developmental stage of differentiation or level of expression of the relevant CD molecule.

Without limiting the present invention to any one theory or mode of action, thymocytes are hematopoietic progenitor cells present in the thymus. They are classified into a number of distinct maturation stages based on the expression of cell surface markers. The earliest thymocyte stage is the “double negative” stage (i.e. negative for both CD4 and CD8), which is also described as lineage-negative, and which can be divided into four substages. The next major stage is the “double positive” stage (i.e. positive for both CD4 and CD8). The final stage in maturation is the single positive stage (positive for either CD8 or CD8).

Thymocytes are derived from bone marrow hematopoietic progenitor cells. Following thymus entry, progenitors proliferate to generate an early lymphoid progenitor population. This step is followed by the generation of CD4/CD8 thymocytes which migrate from the cortico-medullary junction toward the thymus capsule. In addition to proliferation, differentiation and T lineage commitment occurs within the CD4/CD8 thymocyte population. Commitment, or loss of alternative lineage potentials (such as myeloid, B, and NK lineage potentials), also occurs at this stage. Following T lineage commitment, thymocytes undergo β-selection.^{[6]} The ability of T cells to recognize foreign antigens is mediated by the T cell receptor, which is a surface protein able to recognize short protein peptides that are presented by MHC.

Unlike most genes, which have a stable sequence in each cell which expresses them, the T cell receptor is made up of a series of alternative gene fragments. In order to create a functional T cell receptor, the double negative thymocytes undergo TCR gene rearrangement. TCR rearrangement occurs in two steps. First the TCRβ chain is rearranged at the CD4⁻/CD8⁻ stage of T cell development. The TCRβ chain is paired with the pre-Tα to generate the pre-TCR. The cellular disadvantage in the rearrangement process is that many of the combinations of the T cell receptor gene fragments are non-functional. To eliminate thymocytes which have made a non-functional T cell receptor, only cells that have successfully rearranged the beta chain to produce a functional pre-TCR are allowed to develop beyond the CD4⁻/CD8⁻ stage. Cells that fail to produce a functional pre-TCR are eliminated by apoptosis.

Following β-selection thymocytes differentiate to CD4+CD8+ double positive cells, which then undergo TCRα rearrangement, resulting in completely assembled TCR. However many of these T cell receptors will still be non-functional, due to an inability to bind MHC. Accordingly the next major stage of thymocyte development is positive selection, wherein only those thymocytes which express a T cell receptor capable of binding MHC are kept.

The positively selected double positive thymocytes then undergo lineage commitment, maturing into a CD8⁺ T cell or a CD4⁺ T cell. Thereafter negative selection occurs in order to eliminate autoreactive thymocytes. Once the maturation process has been completed, the T cells exit the thymus and enter the peripheral blood stream.

In relation to T regulatory cells, these are selected at the double positive stage by their interaction with the cells within the thymus, begin the transcription of Foxp3 to become T_{reg} cells, although they may not begin to express Foxp3 until the single-positive stage, at which point they are functional T_{reg}s. T_{reg} do not exhibit the limited TCR expression of NKT or γδ T cells and exhibit a larger TCR diversity than effector T cells, biased towards self-peptides. The process of T_{reg} selection is determined by the affinity of interaction with a self-peptide MHC complex. Selection to become a T_{reg} is a “Goldilocks” process. Specifically, a T cell that receives very strong signals will undergo apoptotic death while a cell that receives a weak signal will survive and be selected to become an effector cell. If a T cell receives an intermediate signal, then it will become a regulatory cell. Due to the stochastic nature of the process of T cell activation, all T cell populations with a given TCR will end up with a mixture of T_{eff} and T_{reg}—the relative proportions determined by the affinities of the T cell for the self-peptide-MHC.

Natural killer (NK) cells are a heterogeneous group of T cells that share properties of both T cells and natural killer (NK) cells. Many of these cells recognise the non-polymorphic CD1d molecule, an antigen-presenting molecule that binds self- and foreign lipids and glycolipids. They constitute only approximately 0.1% of all peripheral blood T cells. NK cells co-express an αβ T cell receptor (TCR), but also express a variety of molecular markers that are typically associated with NK cells, such as NK1.1. The best-known NK cells differ from conventional αβ T cells in that their TCRs are far more limited in diversity (‘invariant’ or ‘Type 1’ NK). They and other CD1d-restricted T cells (‘Type 2’ NK) recognise lipids and glycolipids presented by CD1d molecules, a member of the CD1 family of antigen-presenting molecules, rather than peptide-MHC complexes. As such, NK cells are known to be important in recognizing glycolipids from organisms such as mycobacterium, which cause tuberculosis.

B cell development occurs through several stages, each stage representing a change in the genome content at the antibody loci. An antibody is composed of two identical light and two identical heavy chains, and the genes specifying them are found in the ‘V’ (Variable) region and the ‘C’ (Constant) region. In the heavy-chain ‘V’ region there are three segments; V, D, and J, which recombine randomly, in a process called VDJ recombination, to produce a unique variable domain in the immunoglobulin in each individual B cell. Similar rearrangements occur for light-chain ‘V’ region except that there are only two segments involved: V and J. The table below describes the process of immunoglobulin formation at the different stages of B cell development.

| Stage | Heavy chain | Light chain | Ig |
|---|---|---|---|
| Progenitor (or pre- | germline | germline | — |
| pro) B cells | | | |
| Early Pro (or pre-pre)- | undergoes D-J | germline | — |
| B cells | rearrangement | | |
| Late Pro (or pre-pre)- | undergoes V-DJ | germline | — |
| B cells | rearrangement | | |
| Large Pre-B cells | is VDJ rearranged | Germline | IgM in cytoplasm and |
| | | | surface (IgH + pseudo light |
| | | | chain) |
| Small Pre-B cells | is VDJ rearranged | undergoes V-J | IgM in cytoplasm and |
| | | rearrangement | surface |
| Immature B cells | is VDJ rearranged | VJ rearranged | IgM on surface |
| Mature B cells | is VDJ rearranged | VJ rearranged | IgM and IgD on surface |

When the B cell fails in any step of the maturation process, it will die by clonal deletion. B cells are continuously produced in the bone marrow. Like T cells, immature B cells are tested for auto-reactivity by the immune system before leaving the bone marrow. In the bone marrow central tolerance is produced. The immature B cells whose B cell receptors bind too strongly to self antigens will not be allowed to mature. If B cells are found to be highly reactive to self, three mechanisms can occur.
Clonal deletion: the removal, usually by apoptosis, of B cells of a particular self antigen specificity.
Receptor editing: The receptors of self reactive B cells are given an opportunity to rearrange their conformation. This process occurs via the continued expression of the Recombination activating gene. Through the help of RAG, receptor editing involves light chain gene rearrangement of the B cell receptor. If the receptor editing fails to produce a receptor that is less autoreactive, apoptosis will occur.
Anergy: B cells enter a state of permanent unresponsiveness when they bind with weakly cross-linking self antigens that are small and soluble.

B cell types include:
Plasma B cells (also known as plasma cells, plasmocytes, and effector B cells) are large B cells that have been exposed to antigen and produce and secrete large amounts of antibodies. These are short-lived cells and undergo apoptosis when the inciting agent that induced immune response is eliminated. This occurs because of cessation of continuous exposure to various colony-stimulating factors, which is required for survival.
Memory B cells are formed from activated B cells that are specific to the antigen encountered during the primary immune response. These cells are able to live for a long time and can respond quickly following a second exposure to the same antigen.
B-1 cells express IgM in greater quantities than IgG and their receptors show polyspecificity, meaning that they have low affinities for many different antigens. Polyspecific immunoglobulins often exhibit a preference for other immunoglobulins, self antigens, and common bacterial polysaccharides.
B2 cells
Marginal-zone B cells
Follicular B cells
Regulatory B cells are B-cells involved in immune regulation. Subsets of Bregs are found both within the B-1 and B-2 cell population. The two best-described phenotypes are the B10 (CD5+CD1d+) subset and the CD24+CD38+ subset in humans.

Reference to a CD4⁺ and/or CD8⁺ or CD25⁺“lymphocyte” should be understood as a reference to a lymphocyte at any differentiative stage of development including, but not limited to, double positive and single positive thymocytes and mature T cells, including naïve, memory and activated T cells and NK cells. Still without limiting the present invention in any way, whereas most T cells will express an αβ T cell receptor, a subpopulation of γδ T cell receptor cells have been determined to also express CD4 or CD8. Accordingly, any lymphocyte, whether γδ or αβ, should be understood to fall within the scope of the method of the present invention if it expresses one or both of CD4 or CD8. Similarly, reference to CD19⁺ lymphocytes should be understood to refer to B cells at any stage of differentiation.

In another embodiment, said mononuclear cell is a lymphocyte.

According to this embodiment there is provided a method of generating mammalian multilineage potential cells, said method comprising establishing an in vitro cell culture which proportionally comprises:
(i) 10-40% v/v, or functionally equivalent proportion thereof, of a lymphocyte suspension, which lymphocytes express CD4, CD8, CD25, CD19 or CD20;
(ii) 5-40% v/v, or functionally equivalent proportion thereof, of an approximately 5%-85% albumin solution; and
(iii) 30-80% v/v, or functionally equivalent proportion thereof, of a cell culture medium
   wherein said cell culture is maintained for a time and under conditions sufficient to induce the transition of said monocytes cells to a cell exhibiting multilineage differentiative potential.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In another embodiment, said mononuclear cell suspension is 15% v/v or functionally equivalent proportion thereof, said 5-85% albumin solution is used at 15% v/v or functionally equivalent proportion thereof and said culture medium is 70% v/v or functionally equivalent proportion thereof.

In one embodiment, said lymphocytes are double positive CD4⁺/CD8⁺ thymocytes.

In another embodiment, said lymphocytes are single positive CD4⁺ or CD8⁺ T cells.

In still another embodiment, said lymphocytes are CD8⁺ NK cell.

In yet still another embodiment, said lymphocytes are CD25⁺ T regulatory cells.

In still yet another embodiment, said lymphocytes are CD19⁺ B cells.

In still another embodiment, said mononuclear cells are CD20+ cells.

It should be understood that the mononuclear cells of the present invention may be sourced from any suitable tissue, including peripheral blood and the spleen.

In still another embodiment, said mononuclear cells are derived from the peripheral blood.

According to this embodiment there is provided a method of generating mammalian multilineage potential cells, said method comprising establishing an in vitro cell culture which proportionally comprises:
(i) 10-40% v/v, or functionally equivalent proportion thereof, of a peripheral blood derived monocyte suspension, which mononuclear cells express CD4, CD8, CD25, CD19 or CD20;
(ii) 5-40% v/v, or functionally equivalent proportion thereof, of an approximately 5%-85% albumin solution; and
(iii) 30-80% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In another embodiment, said mononuclear cell suspension is 15% v/v or functionally equivalent proportion thereof, said 5-85% albumin solution is used at 15% v/v or functionally equivalent proportion thereof and said culture medium is 70% v/v or functionally equivalent proportion thereof.

In one embodiment, said mononuclear cells are lymphocytes.

In still another embodiment, said lymphocytes are single positive CD4⁺ or CD8⁺ T cells, CD8⁺ NK cells, CD25⁺ T cells, CD19⁺ B cells or CD20⁺ B cells.

As detailed hereinbefore, it has been determined that a mature somatic cell, specifically a mononuclear cell such as a lymphocyte, can be induced to transition into a state of multilineage differentiation potential. Accordingly, reference to a cell exhibiting “multilineage differentiation potential” or “multilineage potential” should be understood as a reference to a cell which exhibits the potentiality to develop along more than one somatic differentiative path. For example, the cell may be capable of generating a range of somatic cell types, such cells usually being referred to as pluripotent or multipotent. These cells exhibit commitment to a more limited range of lineages than a totipotent cell, the latter being a cell which can develop in any of the differentiation directions inherently possible including all the somatic lineages and the gametes. Without limiting the present invention to any one theory or mode of action, to the extent that a stem cell is derived from post-natal tissue, it is also often referred to as an “adult stem cell”. Many cells that are classically termed “progenitor” cells or “precursor” cells may also fall within the scope of the definition of “multilineage differentiation potential” on the basis that, under appropriate stimulatory conditions, they can give rise to cells of more than one somatic lineage. To the extent that reference to “stem cell” is made herein in terms of the cells generated by the method of the invention, this should be understood as a reference to a cell exhibiting multilineage differentiative potential as herein defined.

In one embodiment of the present invention, it has been determined that CD4, CD8, CD25, CD19 or CD20 mononuclear cells can be induced to transition to a multilineage differentiative potential phenotype which exhibits potentiality to differentiate along multiple different lineages, such as a haematopoietic lineage or a mesenchymal lineage. For example, under appropriate stimulation the subject multipotential cell can be directed to differentiate down a haematopoietic lineage including mononuclear haematopoietic cells (such as lymphocytes or monocytes), polymorphonuclear haematopoietic cells (such as neutrophils, basophils or eosinophils), red blood cells or platelets, or along a mesenchymal lineage such as connective tissues such as bone, cartilage, smooth muscle, tendon, ligament, stroma, marrow, dermis and fat. In the presence of appropriate stimuli, these cells can also be induced to differentiate along other lineages, such as neuronal lineages. It should also be understood that although all of the multilineage potential cells which are generated in accordance with the method of the present invention may be derived from one of a number of different starting population, they all exhibit the potentiality to differentiate along multiple lineages. Without limiting the present invention to any one theory or mode of action, the multilineage cells generated from the CD4, CD8, CD25, CD19 or CD20 starting cells of the present invention exhibit unique phenotypic profiles. Although all of these cells exhibit multipotency, these cells may exhibit functional differences in terms of their predisposition, if any, to differentiate along a particular lineage in the absence of specific extracellular stimuli. However, where specific stimuli are provided, differentiation can be directed along any desired lineage.

A one embodiment of the present invention is therefore directed to a method of generating mammalian multilineage potential cells, said method comprising establishing an in vitro cell culture which proportionally comprises:
(i) 10-40% v/v, or functionally equivalent proportion thereof, of a mononuclear cell suspension, which mononuclear cells express CD4, CD8, CD25, CD19 or CD20;
(ii) 5-40% v/v, or functionally equivalent proportion thereof, of an approximately 5%-85% albumin solution; and
(iii) 30-80% v/v, or functionally equivalent proportion thereof, of a cell culture medium
   wherein said cell culture is maintained for a time and under conditions sufficient to induce the transition of said mononuclear cells to a cell exhibiting multilineage differentiative potential, which multilineage potential cell exhibits haematopoietic and/or mesenchymal potential

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In another embodiment, said mononuclear cell suspension is 15% v/v or functionally equivalent proportion thereof, said 5-85% albumin solution is used at 15% v/v or functionally equivalent proportion thereof and said culture medium is 70% v/v or functionally equivalent proportion thereof.

In another embodiment, said CD4⁺ derived multilineage potential cell expresses CD44⁺ and CD45⁺.

In still another embodiment, said CD8⁺ derived multilineage potential cell expresses CD45⁺ and CD47⁺.

In yet another embodiment, said CD25⁺ derived multilineage potential cell expresses CD23⁺.

In still yet another embodiment, said CD19⁺ derived multilineage potential cell expresses CD44⁺ and CD45⁺.

More preferably, said haematopoietic potentiality is the potentiality to differentiate to a lymphocyte, monocyte, neutrophil, basophil, eosinophil, red blood cell or platelet and said mesenchymal potentiality is the potentiality to differentiate to a cell of the bone, cartilage, smooth muscle, tendon, ligament, stroma, marrow, dermis or fat.

The terms “mammal” and “mammalian” as used herein include humans, primates, livestock animals (e.g. horses, cattle, sheep, pigs, donkeys), laboratory test animals (e.g. mice, rats, guinea pigs), companion animals (e.g. dogs, cats) and captive wild animal (e.g. kangaroos, deer, foxes). Preferably, the mammal is a human or a laboratory test animal Even more preferably, the mammal is a human.

Reference to inducing the “transition” of a CD4, CD8, CD25, CD19 or CD20 mononuclear cell, such as a monocyte, to a multilineage potential phenotype should be understood as a reference to inducing the genetic, morphologic and/or functional changes which are required to change a somatic phenotype to a multilineage potential phenotype of the type defined herein.

In terms of inducing the in vitro de-differentiation of a CD4, CD8, CD25, CD19 or CD20 mononuclear cell to a multilineage potential cell, this can be achieved either in the context of small scale in vitro tissue culture or large scale bioreactor production.

As detailed hereinbefore, it has been determined that the transition of a CD4, CD8, CD25, CD19 or CD20 mononuclear cell to a cell of multilineage potential can be achieved in vitro by subjecting said cells to a unique cell culture regime. Specifically, a starting sample of mononuclear cells are cultured in specific proportions together with albumin and a cell culture medium. This is a particular advantage of the present method since unlike most cell culture systems, the establishment of the present culture is not based on culturing a specific concentration of cells, which entails determination of cell numbers and appropriate adjustment of cell concentration, but is based on designing the culture around volume proportions, irrespective of the actual number of cells within that volume. This renders the present method very simple and routine to perform based on whatever starting volume of CD4, CD8, CD25, CD19 or CD20 mononuclear cells are either available or convenient to work with.

The in vitro cell culture system of the present invention is therefore established around the starting volume of CD4, CD8, CD25, CD19 or CD20 mononuclear cell suspension. Reference to “suspension” should be understood as a reference to a sample of non-adherent cells. These cells may be contained in any suitable medium such as an isotonic solution (e.g. PBS, saline, Hank's balanced salt solution or other balanced salt solution variations), cell culture medium, bodily fluid (e.g. serum) or the like which will maintain the cells in a viable state. The subject cells may have undergone enrichment or treatment by other methods, such as positive or negative magnetic bead separation, which would result in the final suspension of CD4, CD8, CD25, CD19 or CD20 mononuclear cells being contained in any one of a variety of different isotonic solutions, depending upon the nature of the method which is utilised. Irrespective of the actual concentration of cells which are obtained, any suitable volume of this suspension can be used to establish the culture of the present invention. This volume will be selected based on the type of culture system which is sought to be used. For example, if one is culturing in a flask-based system, bag-based system or roller bottle-based system, it is likely that smaller volumes, up to about one litre, will form the totality of the cell culture. However, in the context of a bioreactor, significantly larger volumes of cell culture can be accommodated and thereby larger starting volumes can be used. It is well within the skill of the person in the art to determine an appropriate final cell culture volume for use in the context of the particular cell culture system which will be utilised.

In terms of initially establishing the cell culture of the present invention, the final volume of the cell culture which will undergo culturing comprises about 15% v/v of a CD4, CD8, CD25, CD19 or CD20 mononuclear cell suspension together with about 15% v/v of a 5%-85% albumin solution and about 70% v/v of a cell culture medium. As detailed herein, references to these percentage values are approximate to the extent that some deviation from these specific percentages is acceptable and provides a functionally equivalent proportion. It is well within the skill of the person in the art to determine, based on the very simple and routine nature of the exemplified culturing system, to what extent some deviation from the above percentage values is enabled. For example, it is to be expected that from about 20% to 40% v/v of the mononuclear cell suspension and 5-40% of the 5%-85% albumin solution may be effective, in particular 10%-40%, 15%-40%, 20%-40% or about 15%. In relation to the subject albumin solution, a solution of from about 4% to 90%, or 5%-86% or preferably 5%-7% may be equally effective. 30%-60% of the cell culture medium may be used, for example 30%-40%.

Without limiting the present invention in any way, it has been determined that an albumin concentration across a very wide range is effective in the method of the invention. Accordingly, one may use a concentration range of 5%-85%, 5%-80%, 5%-75%, 5%-70%, 5%-65%, 5%-60%, 5%-50%, 5%-45%, 5%-40%, 5%-35%, 5%-30%, 5%-25%, 5%-20%, 5%-15%, 5%-10%. In one embodiment, said concentration is 5%-20%.

Accordingly, one embodiment of the present invention is therefore directed to a method of generating mammalian multilineage potential cells, said method comprising establishing an in vitro cell culture which proportionally comprises:
(i) 20-40% v/v, or functionally equivalent proportion therefore of a mononuclear cell suspension, which mononuclear cells express CD4, CD8, CD25, CD19 or CD20;
(ii) 20-40% v/v, or functionally equivalent proportion thereof, of an approximately 5%-20% albumin solution; and
(iii) 30-50% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In one embodiment, said CD4⁺ or CD8⁺ mononuclear cell suspension is 30% v/v or functionally equivalent proportion thereof, said 5-85% albumin solution is used at 40% v/v or functionally equivalent proportion thereof and said culture medium is 30% v/v or functionally equivalent proportion thereof.

In another embodiment, said CD19⁺ mononuclear cell suspension is 40% v/v or functionally equivalent proportion thereof, said 5-85% albumin solution is used at 20% v/v or functionally equivalent proportion thereof and said culture medium is 40% v/v or functionally equivalent proportion thereof.

In still another embodiment, said CD25⁺ mononuclear cell suspension is 20% v/v or functionally equivalent proportion thereof, said 5-85% albumin solution is used at 40% v/v or functionally equivalent proportion thereof and said culture medium is 40% v/v or functionally equivalent proportion thereof.

In still another embodiment, said CD20⁺ mononuclear cell suspension is 20% v/v or functionally equivalent proportion thereof, said 5-85% albumin solution is used at 40% v/v or functionally equivalent proportion thereof and said culture medium is 40% v/v or functionally equivalent proportion thereof.

In yet another embodiment, said mononuclear cell suspension is 15% v/v or functionally equivalent proportion thereof, said 5-85% albumin solution is used at 15% v/v or functionally equivalent proportion thereof and said culture medium is 70% v/v or functionally equivalent proportion thereof.

In another embodiment, said albumin solution concentration is 5%, 6%, 7%, 8%, 9%, 10%, 11%, 12%, 13%, 14%, 15%, 16%, 17%, 18%, 19% or 20%.

The present invention should not be limited by reference to strict adherence to percentage values detailed herein, in particular in relation to the above embodiments, but includes within its scope variation to these percentages which retain the functionality of the present invention and which can be routinely and easily assessed by the person of skill in the art.

As detailed hereinbefore, the concentration of CD4, CD8, CD25, CD19 or CD20 mononuclear cells within the starting cell suspension can be any number of cells. Whether that cell number is relatively low or relatively high, the important aspect of the present invention is that the starting cell suspension is 15-40% v/v of the total volume of the starting cell culture, irrespective of the concentration of cells within that suspension. Nevertheless, in a preferred embodiment, although there is neither a lower limit nor an upper limit to the starting cell concentration, it is suggested that the cell number should not be so high that there is insufficient surface area in the culture container for these mononuclear cells to adhere to during culture. Although the method will nevertheless succeed in producing cells exhibiting multilineage differentiative potential, to the extent that the starting cell concentration is so high that there may be insufficient surface area for these cells to adhere, one might simply observe that those cells unable to adhere do not de-differentiate to a stem cell and thereby although the method is effective it is not optimally efficient. Accordingly, in this regard, from the point of view of maximizing efficiency one may wish to ensure that the cell concentration which forms part of the starting cell culture is cultured within an environment that all of the cells present are able to adhere to the particular tissue culture container which is selected for use. For example, where one is using a culture bag container, a cell concentration of not more than 10⁶ cells/ml is suitable.

In terms of the albumin solution which is used, a 6% albumin solution is commonly commercially available but may otherwise be made up in any suitable isotonic solution, such as saline. It should be understood that reference to “albumin” is intended as a reference to the group of globular proteins which are soluble in distilled water and solutions of half-saturated ammonium sulphate, but insoluble in fully saturated ammonium sulphate solution. For example, serum albumin, which is a major protein of serum, may be used in the context of the method of the present invention. However, it should be understood that any albumin molecule may be utilised such as lactalbumin or ovalbumin. It should also be understood that any synthetic recombinant or derivative forms of albumin may also be used in the method of the present invention. It would be appreciated by the person of skill in the art that by using the 6% albumin solution, for example, in the proportion of 15% v/v of the starting culture volume of the present invention, an effective concentration of 0.9% albumin is achieved.

The remainder of the starting culture volume is comprised of cell culture medium, this forming, preferably, 30-80% v/v of the starting cell culture volume. Reference to “cell culture medium” should be understood as a reference to a liquid or gel which is designed to support the growth of mammalian cells, in particular medium which will support stem cell culturing. To this end, any suitable cell culture medium may be used including minimal media, which provide the minimum nutrients required for cell growth, or enriched media, which may contain additional nutrients to promote maintenance of viability and growth of mammalian cells. Examples of media suitable for use include DMEM and RPMI. One may also use a supplementary minimal medium which contains an additional selected agent such as an amino acid or a sugar to facilitate maintenance of cell viability and growth. The medium may also be further supplemented with any other suitable agent, for example antibiotics. In another example the cell culture medium is supplemented with insulin in order to further support cell viability and growth. It should be understood that reference to the 30-80% v/v cell culture medium is a stand alone requirement which is not impacted upon by the nature of the solutions, whether they be isotonic solutions such as saline or minimal culture media, which the starting CD4, CD8, CD25, CD19 or CD20 mononuclear cells or albumin are suspended in. It is in fact a particular advantage of the present invention that irrespective of the nature of the solution within which the mononuclear cells are initially suspended, prior to their introduction to the culture system of the present invention, or in which the albumin is dissolved, the requirement for the 30-80% v/v cell culture medium as a percentage of the total volume of the starting cell culture population remains unchanged.

In one embodiment, said cell culture additionally comprises 10 mg/L insulin.

As detailed hereinbefore, the method of the present invention is predicated on culturing a population of CD4, CD8, CD25, CD19 or CD20 mononuclear cells in specific proportions together with a cell culture medium and a 5%-85% albumin solution to induce de-differentiation of the mononuclear cells to a mesenchymal/haematopoietic stem cell phenotype. Said CD4, CD8, CD25, CD19 or CD20 mononuclear cells are cultured in vitro until such time as the subject stem cell phenotype is achieved. In one embodiment, a culture period of 3-8 days, in particular 4-7 days, has been determined to be appropriate for generating the subject stem cells. It would be appreciated that it is well within the skill of the person in the art to sample the in vitro cultured cells to determine whether or not the requisite extent of de-differentiation has occurred. It would also be well within the skill of the person in the art to determine the most appropriate conditions under which to culture the cells both in terms of temperature and CO₂ percentage. Without limiting the present invention to any one theory or mode of action, it has been determined that 4 to 5 days of incubation is particularly suitable when culturing human CD4, CD8, CD25, CD19 or CD20 mononuclear cells. The culturing can proceed under conditions as deemed appropriate to maintain good cell viability and growth over the culture period of several days. To this end, it would be appreciated that establishing appropriate cell culture conditions is a matter of routine procedure for the person of skill in the art.

Accordingly, in one embodiment there is provided a method of generating human multilineage potential cells, said method comprising establishing an in vitro cell culture which proportionally comprises:
(i) 10-40% v/v, or functionally equivalent proportion thereof, of a human peripheral blood mononuclear cell suspension, which mononuclear cells express CD4, CD8, CD25, CD19 or CD20;
(ii) 5-40% v/v, or functionally equivalent proportion thereof, of an approximately 5%-85% albumin solution; and
(iii) 30-80% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In another embodiment, said mononuclear cell suspension is 15% v/v or functionally equivalent proportion thereof, said 5-85% albumin solution is used at 15% v/v or functionally equivalent proportion thereof and said culture medium is 70% v/v or functionally equivalent proportion thereof.

In one embodiment, said albumin solution is 5%-20%, preferably 5%-15%.

In one embodiment, said cell culture additionally includes 10 mg/L human insulin or functional fragment or equivalent thereof.

In another embodiment, said cells are culture for 4 to 7 days, in particular 4 to 5 days or 3 to 6 days.

As detailed hereinbefore, the present invention is performed in vitro on an isolated population of CD4, CD8, CD25, CD19 or CD20 mononuclear cells. To this end, it should be understood that the subject cells may have been freshly isolated from an individual (such as an individual who may be the subject of treatment) or they may have been sourced from a non-fresh source, such as from a culture (for example, where cell numbers were expanded and/or the cells were cultured so as to render them receptive to differentiation signals) or a frozen stock of cells (for example, an established T cell line), which had been isolated at some earlier time point either from an individual or from another source. It should also be understood that the subject cells may have undergone some other form of treatment or manipulation, such as but not limited to enrichment or purification, modification of cell cycle status or the formation of a cell line. Accordingly, the subject cell may be a primary cell or a secondary cell. A primary cell is one which has been isolated from an individual. A secondary cell is one which, following its isolation, has undergone some form of in vitro manipulation, such as the preparation of a cell line, prior to the application of the method of the invention. It should also be understood that the starting CD4, CD8, CD25, CD19 or CD20 mononuclear cell population may be relatively pure or it may be part of a heterogeneous cell population, such as a population of peripheral blood cells. This is discussed further hereafter.

In a related aspect, it should be understood that the method of the present invention can also be adapted to induce the differentiation of the multilineage potential cells (MLPCs) which are produced by the method of the present invention to more mature phenotypes. For example, in the context of one embodiment of the present invention, haematopoietic stem cells give rise to all the blood cells (e.g. red blood cells, platelets, lymphocytes, monocytes and the granulocytes) while mesenchymal stem cells give rise to a wide variety of connective tissues including bone, cartilage, smooth muscle, tendon, ligament, stroma, marrow, dermis and fat. To the extent that the method of the present invention produces MLPCs with both mesenchymal and haematopoietic potential, the method of the invention can be adapted, either in vitro or in vivo, to include a further step which introduces the subject MLPC population to the specific stimuli required to effect partial or full differentiation along the lineage of interest.

It should also be understood that although this additional directed differentiation event is conveniently performed in vitro, it could also be achieved in vivo. This is discussed in more detail hereinafter. However, a specific in situ environment may also conveniently provide the range of signals required to direct the differentiation of an MLPC along a particular lineage.

Reference to “MLPC-derived cells” should therefore be understood as a reference to cell types which are more differentiated than a MLPC and which have arisen from said MLPC. These cells will correspond to cells of the lineages to which the MLPC is known to give rise, such as blood cells in the context of haematopoietic stem cells and connective tissue in the context of mesenchymal stem cells. It should be understood that the subject MLPC-derived cell may be a more differentiated precursor cell which is irreversibly committed to differentiating along a particular subgroup of cellular lineages, such as a haematopoietic stem cell or a mesenchymal stem cell, or it may correspond to a partially or terminally differentiated form of a specific cellular lineage, such as a red blood cell, lymphocyte or the like. It should therefore be understood that the cells falling within the scope of this aspect of the present invention may be at any post-MLPC differentiative stage of development. As detailed hereinbefore, this further differentiation may occur constitutively or it may require one or more further signals. These signals may be provided either in vitro, such as in the context of small scale in vitro tissue culture or large scale bioreactor production, or in an in vivo microenvironment, such as if a precursor cell is transplanted into an appropriate tissue microenvironment to enable its further differentiation.

Accordingly, in a related aspect of the present invention there is provided a method of facilitating the generation of a mammalian MLPC-derived cell, said method comprising:

(i) establishing an in vitro cell culture which proportionally comprises:
(a) 10-40% v/v, or functionally equivalent proportion thereof, of a mononuclear cell suspension, which mononuclear cells express CD4, CD8, CD25, CD19 or CD20;
(b) 5-40% v/v, or functionally equivalent proportion thereof, of an approximately 5%-85% albumin solution; and
(c) 30-80% v/v, or functionally equivalent proportion thereof, of a cell culture medium
   wherein said cell culture is maintained for a time and under conditions sufficient to induce the transition of said mononuclear cells to a MLPC; and optionally
   (ii) contacting the MLPC of step (i) with a stimulus to direct the differentiation of said MLPC to a MLPC-derived phenotype.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In another embodiment, said mononuclear cell suspension is 15% v/v or functionally equivalent proportion thereof, said 5-85% albumin solution is used at 15% v/v or functionally equivalent proportion thereof and said culture medium is 70% v/v or functionally equivalent proportion thereof.

In one embodiment, said CD4⁺ and/or CD8⁺ mononuclear cell is a lymphocyte, more preferably a peripheral blood derived CD4 or CD8 single positive T cell.

In still another embodiment, said lymphocyte is a CD8⁺ NK cell.

In yet still another embodiment, said lymphocyte is a CD25⁺ T regulatory cell.

In still yet another embodiment, said lymphocyte is a CD19⁺ B cell.

In still yet another embodiment, said lymphocyte is a CD20⁺ B cell.

In another embodiment, said albumin is 5%-20%.

In yet another embodiment, said MLPC exhibits both haematopoietic and mesenchymal potential.

According to this embodiment there is therefore preferably provided a method of facilitating the generation of a mammalian MLPC-derived cell, said method comprising:

(i) establishing an in vitro cell culture which proportionally comprises
(a) 10-40% v/v, or functionally equivalent proportion thereof, of a mononuclear cell suspension, which mononuclear cells express CD4, CD8, CD25, CD19 and CD20;
(b) 5-40% v/v, or functionally equivalent proportion thereof, of an approximately 5%-85% albumin solution; and
(c) 30-80% v/v, or functionally equivalent proportion thereof, of a cell culture medium
   wherein said cell culture is maintained for a time and under conditions sufficient to induce the transition of said mononuclear cells to a MLPC; and optionally
   (ii) contacting the MLPC step (i) with a stimulus to direct the differentiation of said MLPC to a haematopoietic or mesenchymal phenotype.

In one embodiment, said mononuclear cell suspension is 20-40% v/v or functionally equivalent proportion thereof, said 5-85% albumin solution is used at 15-40% v/v or functionally equivalent proportion thereof and said culture medium is 30-80% v/v or functionally equivalent proportion thereof.

In another embodiment, said mononuclear cell suspension is 15% v/v or functionally equivalent proportion thereof, said 5-85% albumin solution is used at 15% v/v or functionally equivalent proportion thereof and said culture medium is 70% v/v or functionally equivalent proportion thereof.

Still more preferably said haematopoietic stem cell-derived cell is a red blood cell, platelet, lymphocyte, monocyte, neutrophil, basophil or eosinophil.

In another preferred embodiment, said mesenchymal stem cell-derived cell is a connective tissue cell such as a cell of the bone, cartilage, smooth muscle, tendon, ligament, stroma, marrow, dermis or fat.

In the context of this aspect of this invention, it should be understood that there may be produced both cellular aggregates such as tissues (for example, muscular or dermal tissue), or cell suspensions (for example, haematopoietic cell suspensions).

As detailed hereinbefore, the present invention is predicated on the determination that stem cells can be generated from CD4, CD18, CD25, CD19 or CD20 mononuclear cells. To this end, it should be understood that this may be achieved either in the context of directing the transition of all the CD4, CD8, CD25, CD19 and CD20 cells of a starting population or in the context of directing the transition of a subpopulation of the starting population of these somatic cells. This is likely to depend, for example, on the purity and/or heterogeneity of the starting cell population. Still further, the culture system of the invention may result in the production of a heterogeneous population of cells. This may occur, for example, if not all the cells of the starting population transition to a MLPC phenotype or if not all the MLPC cells are thereafter induced to differentiate to a more mature and homogeneous phenotype. This being the case, since not all the cells of the starting population may necessarily differentiate to the MLPC phenotype or MLPC-derived phenotype, and the MLPC-derived cellular output which is obtained may itself be heterogeneous, the method of the invention may require the application of a screening and selection step to identify and isolate cells exhibiting the desired phenotype. Identification methods would be well known to the person of skill in the art and include, but are not limited to:

### (i) Detection of Cell Lineage Specific Structures.

Detection of cell lineage specific structures can be performed, for example, via light microscopy, fluorescence affinity labelling, fluorescence microscopy or electron microscopy, depending on the type of structure to be identified. Light microscopy can be used to detect morphologic characteristics such as lymphocyte vs polymorphonuclear vs red blood cell nuclear characteristics or multinucleate skeletal muscle cells. In another example, mononuclear cells which are about 10-30 μm in diameter, with round or rod-shaped morphology characteristic of immature cardiomyocytes can be identified. Electron microscopy can be used to detect structures such as sarcomeres, X-bands, Z-bodies, intercalated discs, gap junctions or desmosomes. Fluorescence affinity labelling and fluorescence microscopy can be used to detect cell lineage specific structures by fluorescently labelling a molecule, commonly an antibody, which specifically binds to the structure in issue, and which is either directly or indirectly conjugated to a fluorophore. Automated quantitation of such structures can be performed using appropriate detection and computation systems.

### (ii) Detection of Cell Lineage Specific Proteins.

Detection of cell lineage specific proteins, such as cell surface proteins or intracellular proteins, may be conveniently effected via fluorescence affinity labelling and fluorescence microscopy, for example. Specific proteins can be detected in both whole cells and tissues. Briefly, fluorescently labelled antibodies are incubated on fixed cells to detect specific cardiac markers. Alternatively, techniques such as Western immunoblotting or hybridization micro arrays (“protein chips”) may be employed. The proteins which can be detected via this method may be any protein which is characteristic of a specific population of cells. For example, classes of precursor/progenitor cell types can be distinguished via the presence or absence of expression of one or more cell surface molecules. In this regard, this method can be utilised to identify cell types via either a positive or negative selection step based on the expression of any one or more molecules. More mature cells can usually be characterised by virtue of the expression of a range of specific cell surface or intracellular proteins which are well defined in the literature. For example, the differentiative stages of all the haematopoietic cell types have been well defined in terms of cell surface molecule expression patterns. Similarly, muscle cells and other mesenchymal-derived cell types are also well documented in the context of protein expression profiles through the various differentiative stages of development. To this end, the MLPCs of the present invention typically express a range of cell surface markers which are exemplified herein, these being cell surface markers characteristic of monocytic stem cells generally, mesenchymal stem cells, haematopoietic stem cells, multilineage potential cells and neuronal stem cells.
   (iii) Detection of Cell Lineage Specific RNA or DNA.
This method is preferably effected using RT-PCR or real-time (qRT-PCR). Alternatively, other methods, which can be used include hybridization microarray (“RNA chip”) or Northern blotting or Southern blotting. RT-PCR can be used to detect specific RNAs encoding essentially any protein, such as the proteins detailed in point (ii) above, or proteins which are secreted or otherwise not conveniently detectable via the methodology detailed in point (ii). For example, in the context of early B cell differentiation, immunoglobulin gene rearrangement is detectable at the DNA level prior to cell surface expression of the rearranged immunoglobulin molecule.

### (iv) Detection of Cell Lineage Specific Functional Activity.

Although the analysis of a cell population in terms of its functioning is generally regarded as a less convenient method than the screening methods of points (i)-(iii), in some instances this may not be the case. For example, to the extent that one is seeking to generate cardiac cells, one may simply screen, under light microscopy, for cardiac specific mechanical contraction.

It should be understood that in the context of characterising the population of cells obtained via the application of the method of the present invention, any one or more of the techniques detailed above may be utilised.

In terms of either enriching a mature somatic cell population for CD4, CD8, CD25, CD19 or CD20 lymphocytes prior to culturing in accordance with the method of the invention or isolating or enriching a MLPC cell population derived therefrom there are, again, various well known techniques which can be performed. As detailed hereinbefore, antibodies and other cell surface binding molecules, such as lectins, are particularly useful for identifying markers associated with particular cell lineages and/or stages of differentiation. The antibodies may be attached to a solid support to allow for separation. However, other cell separation techniques include those based on differences in physical characteristics (density gradient centrifugation and counter-flow centrifugal elutriation) and vital staining properties (mitochondria-binding dye rhodamine 123 and DNA-binding dye Hoechst 33342).

Procedures for separation may include magnetic separation, using antibody or lectin-coated magnetic beads, affinity chromatography, “panning” with antibody attached to a solid matrix or any other convenient technique. Other techniques providing particularly accurate separation include fluorescence activated cell sorting, this technique also being applicable to the separation of cells based on morphological characteristics which are discernible by forward vs side light scatter. Whereas these techniques can be applied in the context of either positive or negative selection, additional negative selection techniques include, but are not limited to, the site-directed administration of a cytolytic, apoptotic or otherwise toxic agent. This may be most conveniently achieved via the coupling of such an agent to a monoclonal antibody in order to facilitate its directed delivery. In another example, opsonisation with an antibody followed by complement administration may achieve the same outcome.

These techniques can be performed as either a single-step or multi-step protocol in order to achieve the desired level of purification or enrichment.

Since the proliferative capacity of the cells and tissues of the present invention may be essential to a given use, for example to repair damaged tissue, or to test the effects of a therapeutic treatment regime, it may be desirable to screen for cells which are displaying an adequate level of proliferative capacity. Determining the proliferative capacity of cells can be performed by numerous standard techniques. Preferably, determination of proliferation is effected via ³[H]-thymidine or ¹²⁵I-iododeoxyuridine uptake assay. Alternatively, colorimetric assays employing metabolic dyes such as XTT or direct cell counting may be employed to ascertain proliferative capacity. Proliferation capacity can also be evaluated via the expression of cell cycle markers such as Ki-67.

As detailed hereinbefore, the method of the present invention is performed in vitro. In terms of in vitro technology, there is therefore now provided means of routinely and reliably producing MLPC or MLPC-derived cells on either a small scale or on a larger scale. In terms of small scale production, which may be effected in tissue culture flasks or bags for example, this may be particularly suitable for producing populations of cells for a given individual and in the context of a specific condition. In terms of large scale production, the method of the invention provides a feasible means of meeting large scale needs. One means of achieving large scale production in accordance with the method of the invention is via the use of a bioreactor.

Bioreactors are designed to provide a culture process that can deliver medium and oxygenation at controlled concentrations and rates that mimic nutrient concentrations and rates in vivo. Bioreactors have been available commercially for many years and employ a variety of types of culture technologies. Of the different bioreactors used for mammalian cell culture, most have been designed to allow for the production of high density cultures of a single cell type and as such find use in the present invention. Typical application of these high density systems is to produce as the end-product, a conditioned medium produced by the cells. This is the case, for example, with hybridoma production of monoclonal antibodies and with packaging cell lines for viral vector production. However, these applications differ from applications where the therapeutic end-product is the harvested cells themselves, as in the present invention.

Once operational, bioreactors provide automatically regulated medium flow, oxygen delivery, and temperature and pH controls, and they generally allow for production of large numbers of cells. Bioreactors thus provide economies of labour and minimization of the potential for mid-process contamination, and the most sophisticated bioreactors allow for set-up, growth, selection and harvest procedures that involve minimal manual labour requirements and open processing steps. Such bioreactors optimally are designed for use with a homogeneous cell mixture or aggregated cell populations as contemplated by the present invention. Suitable bioreactors for use in the present invention include but are not limited to those described in U.S. Pat. No. 5,763,194, U.S. Pat. Nos. 5,985,653 and 6,238,908, U.S. Pat. No. 5,512,480, U.S. Pat. Nos. 5,459,069, 5,763,266, 5,888,807 and 5,688,687.

With any large volume, long term cell culture, such as where the in vitro directed differentiation of the MLPCs is desired, several fundamental parameters require control. Cultures must be provided with medium that allows for cell viability maintenance, proliferation and differentiation (perhaps in the context of several separate differentiation cultures and conditions) as well as final cell culture preservation. Typically, the various media are delivered to the cells by a pumping mechanism in the bioreactor, feeding and exchanging the medium on a regular basis. The exchange process allows for by-products to be removed from the culture. Growing cells or tissue also requires a source of oxygen. Different cell types can have different oxygen requirements. Accordingly, a flexible and adjustable means for providing oxygen to the cells is a desired component.

Depending on the particular culture, even distribution of the cell population and medium supply in the culture chamber can be an important process control. Such control is often achieved by use of a suspension culture design, which can be effective where cell-to-cell interactions are not important. Examples of suspension culture systems include various tank reactor designs and gas-permeable plastic bags. For cells that do not require assembly into a three-dimensional structure or require proximity to a stromal or feeder layer (such as most blood cell precursors or mature blood cells) such suspension designs may be used.

Efficient collection of the cells at the completion of the culture process is an important feature of an effective cell culture system. One approach for production of cells as a product is to culture the cells in a defined space, without physical barriers to recovery, such that simple elution of the cell product results in a manageable, concentrated volume of cells amenable to final washing in a commercial, closed system cell washer designed for the purpose. Optimally, the system would allow for addition of a pharmaceutically acceptable carrier, with or without preservative, or a cell storage compound, as well as provide efficient harvesting into appropriate sterile packaging. Optimally the harvest and packaging process may be completed without breaking the sterile barrier of the fluid path of the culture chamber.

With any cell culture procedure, a major concern is sterility. When the product cells are to be transplanted into patients (often at a time when the patient is ill or immunocompromised), absence of microorganisms is mandated.

The development of the present invention has now facilitated the development of means for therapeutically or prophylactically treating subjects. In particular, and in the context of the preferred embodiments of the present invention, means for treating patients exhibiting inadequate, insufficient or aberrant haematopoietic or mesenchymal cellular functioning is provided based on administering to these subjects MLPCs or partially or fully differentiated MLPC-derived cells (such as haematopoietic or mesenchymal derived cells) which have been generated according to the method of the present invention;

This method can be applied to a wide range of conditions including, but not limited to haematopoietic disorders, circulatory disorders, stroke, myocardial infarction, hypertension bone disorders, type II diabetes, infertility, damaged or morphologically abnormal cartilage or other tissue, hernia repair, pelvic floor prolapse surgery using supportive mesh and biological scaffolds, cell therapy for other musculoskeletal disorders and replacement of defective supportive tissues in the context of aging, surgery or trauma.

Reference to a condition characterised by “aberrant haematopoietic or mesenchymal cellular functioning” should be understood as a reference to any condition which is due, at least in part, to a defect or unwanted or undesirable outcome in terms of the functioning or development of cells of the haematopoietic or mesenchymal lineages. This may correspond to either a homogeneous or heterogeneous population of cells. Reference to “haematopoietic stem cells”, “haematopoietic stem cell-derived cells”, “mesenchymal stem cells” or “mesenchymal stem cell-derived cells” should be understood to have the same meaning as defined hereinbefore. The subject defect should be understood as a reference to any structural or functional feature of the cell which is either not normal or otherwise undesirable, including the production of insufficient numbers of these cells.

Accordingly, another aspect of the present invention is directed to a method of therapeutically and/or prophylactically treating a condition in a mammal, said method comprising administering to said mammal an effective number of MLPCs or partially or fully differentiated MLPC-derived cells which have been generated according to the method of the present invention.

More particularly, there is provided a method of therapeutically and/or prophylactically treating a condition characterised by aberrant haematopoietic or mesenchymal functioning in a mammal, said method comprising administering to said mammal;
(i) an effective number of haematopoietic stem cells or partially or fully differentiated haematopoietic stem cell-derived cells which have been generated according to the method of the present invention; or
(ii) an effective number of mesenchymal stem cells or partially or fully differentiated mesenchymal stem cell-derived cells which have been generated according to the method of the present invention.

Reference to “administering” to an individual an effective number of the cells of the invention should be understood to as a reference to introducing into the mammal an ex vivo population of cells which have been generated according to the method of the invention. Reference to “administering”, an “agent” should be understood as a reference to introducing into the mammal an effective amount of one or more stimuli which will act on an MLPC, which has been introduced in vivo, to generate an MLPC-derived cell.

In accordance with the present invention, the subject MLPCs or MLPC-derived cells are preferably autologous cells which are identified, isolated and/or differentiated to the requisite phenotype ex vivo and transplanted back into the individual from which they were originally harvested. However, it should be understood that the present invention nevertheless extends to the use of cells derived from any other suitable source where the subject cells exhibit the same major histocompatability profile as the individual who is the subject of treatment. Accordingly, such cells are effectively autologous in that they would not result in the histocompatability problems which are normally associated with the transplanting of cells exhibiting a foreign MHC profile. Such cells should be understood as falling within the definition of “autologous”. For example, under certain circumstances it may be desirable, necessary or of practical significance that the subject cells are isolated from a genetically identical twin. The cells may also have been engineered to exhibit the desired major histocompatability profile. The use of such cells overcomes the difficulties which are inherently encountered in the context of tissue and organ transplants. However, where it is not possible or feasible to isolate or generate autologous cells, it may be necessary to utilise allogeneic stem cells. “Allogeneic” cells are those which are isolated from the same species as the subject being treated but which exhibit a different MHC profile. Although the use of such cells in the context of therapeutics would likely necessitate the use of immunosuppression treatment, this problem can nevertheless be minimised by use of cells which exhibit an MHC profile exhibiting similarity to that of the subject being treated, such as a cellular population which has been isolated/generated from a relative such as a sibling, parent or child. The present invention should also be understood to extend to xenogeneic transplantation. That is, the cells which are generated in accordance with the method of the invention and introduced into a patient, are isolated from a mammalian species other than the species of the subject being treated.

Without limiting the present invention to any one theory or mode of action, even partial restoration of the functioning which is not being provided by the aberrant cellular population will act to ameliorate the symptoms of many conditions. Accordingly, reference to an “effective number” means that number of cells necessary to at least partly attain the desired effect, or to delay the onset of, inhibit the progression of, or halt altogether the onset or progression of the particular condition being treated. Such amounts will depend, of course, on the particular conditions being treated, the severity of the condition and individual patient parameters including age, physical conditions, size, weight, physiological status, concurrent treatment, medical history and parameters related to the disorder in issue. One skilled in the art would be able to determine the number of cells and tissues of the present invention that would constitute an effective dose, and the optimal mode of administration thereof without undue experimentation, this latter issue being further discussed hereinafter. These factors are well known to those of ordinary skill in the art and can be addressed with no more than routine experimentation. It is preferred generally that a maximal cell number be used, that is, the highest safe number according to sound medical judgement. It will be understood by those of ordinary skill in the art, however, that a lower cell number may be administered for medical reasons, psychological reasons or for any other reasons.

As hereinbefore discussed, it should also be understood that although the method of the present invention encompasses within its scope the introduction of transitioned or fully or partially differentiated cells to an individual suffering a condition as herein defined, it is not necessarily the case that every cell of the population introduced to the individual will have acquired the MLPC or MLPC-derived phenotype of interest. For example, where a CD4, CD8, CD25, CD19 or CD20 lymphocyte population has undergone transition to MLPCs and is administered in total, there may exist a proportion of cells which have not undergone transition to a cell exhibiting the requisite phenotype. The same issue can occur in the context of administering a population of MLPC-derived cells, such as specific haematopoietic or mesenchymal populations. The present invention is therefore achieved provided the relevant portion of the cells thereby introduced constitute the “effective number” as defined above. However, in a particularly preferred embodiment the population of cells which have undergone differentiation will be subjected to the identification of successfully differentiated cells, their isolation and introduction to the subject individual. This provides a means for selecting either a heterogeneous population of MLPC-derived cells, such as may occur where mesenchymal-derived connective tissue is induced to develop, or to select out a specific subpopulation of cells for administration, such as red blood cells. The type of method which is selected for application will depend on the nature of the condition being treated. However, it is expected that in general it will be desirable to administer a pure population of cells in order to avoid potential side effects such as teratoma formation. Alternatively, in some instances it may be feasible to subject a population of MLPCs to differentiation and provided that this population, as a whole, are shown to exhibit the requisite functional activity, this population as a whole may be introduced into the subject individual without the prior removal of irrelevant cell types. Accordingly, reference to “an effective number”, in this case, should be understood as a reference to the total number of cells required to be introduced such that the number of differentiated cells is sufficient to produce the level of activity which achieves the object of the invention, being the treatment of the subject condition.

As detailed hereinbefore, MLPC transition is performed in vitro. In this situation, the subject cell will then require introduction into an individual. For example, cell suspensions may be introduced by direct injection or inside a blood clot whereby the cells are immobilised in the clot thereby facilitating transplantation. The cells may also be encapsulated prior to transplantation. Encapsulation is a technique which is useful for preventing the dissemination of cells which may continue to proliferate (i.e. exhibit characteristics of immortality) or for minimising tissue incompatibility rejection issues. However, the usefulness of encapsulation will depend on the function which the transplanted cells are required to provide. For example, if the transplanted cells are required primarily for the purpose of secreting a soluble factor, a population of encapsulated cells will likely achieve this objective. However, if the transplanted cells are required for their contractile properties, for example, the cells will likely be required to integrate with the existing tissue scaffold of the muscle. Encapsulated cells would not be able to do this efficiently.

The cells which are administered to the patient can be administered as single or multiple doses by any suitable route. Preferably, and where possible, a single administration is utilised. Administration via injection can be directed to various regions of a tissue or organ, depending on the type of repair required.

It would be appreciated that in accordance with these aspects of the present invention, the cells which are administered to the patient may take any suitable form, such as being in a cell suspension (e.g. blood cells) or taking the form of a tissue graft (e.g. connective tissue). In terms of generating a single cell suspension, the differentiation protocol may be designed such that it favours the maintenance of a cell suspension. Alternatively, if cell aggregates or tissues form, these may be dispersed into a cell suspension. In terms of utilising a cell suspension, it may also be desirable to select out specific subpopulations of cells for administration to a patient, such as specific mononuclear haematopoietic cells. To the extent that it is desired that a tissue is transplanted into a patient, this will usually require surgical implantation (as opposed to administration via a needle or catheter). Alternatively, a portion, only, of this tissue could be transplanted. In another example, engineered tissues can be generated via standard tissue engineering techniques, for example by seeding a tissue engineering scaffold having the designed form with the cells and tissues of the present invention and culturing the seeded scaffold under conditions enabling colonization of the scaffold by the seeded cells and tissues, thereby enabling the generation of the formed tissue. The formed tissue is then administered to the recipient, for example using standard surgical implantation techniques. Suitable scaffolds may be generated, for example, using biocompatible, biodegradable polymer fibers or foams, comprising extracellular matrix components, such as laminins, collagen, fibronectin, etc. Detailed guidelines for generating or obtaining suitable scaffolds, culturing such scaffolds and therapeutically implanting such scaffolds are available in the literature (for example, refer to Kim S. S. and Vacanti J. P., 1999. Semin Pediatr Surg. 8:119, U.S. Pat. No. 6,387,369 to Osiris, Therapeutics, Inc.; U.S. Pat. App. No. US20020094573A1 to Bell E.).

In accordance with the method of the present invention, other proteinaceous or non-proteinaceous molecules may be co-administered either with the introduction of the subject cells or prior or subsequently thereto. By “co-administered” is meant simultaneous administration in the same formulation or in different formulations via the same or different routes or sequential administration via the same or different routes. By “sequential” administration is meant a time difference of from seconds, minutes, hours or days between the introduction of these cells and the administration of the proteinaceous or non-proteinaceous molecules or the onset of the functional activity of these cells and the administration of the proteinaceous or non-proteinaceous molecule. Examples of circumstances in which such co-administration may be required include, but are not limited to:
(i) When administering non-syngeneic cells or tissues to a subject, there usually occurs immune rejection of such cells or tissues by the subject. In this situation it would be necessary to also treat the patient with an immunosuppressive regimen, preferably commencing prior to such administration, so as to minimise such rejection. Immunosuppressive protocols for inhibiting allogeneic graft rejection, for example via administration of cyclosporin A, immunosuppressive antibodies, and the like are widespread and standard practice.
(ii) Depending on the nature of the condition being treated, it may be necessary to maintain the patient on a course of medication to alleviate the symptoms of the condition until such time as the transplanted cells become integrated and fully functional. Alternatively, at the time that the condition is treated, it may be necessary to commence the long term use of medication to prevent re-occurrence of the damage. For example, where the subject damage was caused by an autoimmune condition (such as occurs in the context of rheumatoid arthritis), the ongoing use of immunosuppressive drugs may be required even when syngeneic stem cells have been used to replace or repair cartilage.

It should also be understood that the method of the present invention can either be performed in isolation to treat the condition in issue or it can be performed together with one or more additional techniques designed to facilitate or augment the subject treatment. These additional techniques may take the form of the co-administration of other proteinaceous or non-proteinaceous molecules, as detailed hereinbefore.

Another aspect of the present invention is directed to the use of a population of MLPCs or MLPC-derived cells, which cells have been generated in accordance with the method of the present invention, in the manufacture of a medicament for the treatment of a condition in a mammal.

Yet another aspect of the present invention is directed to MLPCs or MLPC-derived cells and which have been generated in accordance with the method of the present invention.

Preferably, said MLPCs are haematopoietic or mesenchymal stem cells.

In a related aspect of the present invention, the subject undergoing treatment or prophylaxis may be any human or animal in need of therapeutic or prophylactic treatment. In this regard, reference herein to “treatment” and “prophylaxis” is to be considered in its broadest context. The term “treatment” does not necessarily imply that a mammal is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity of the onset of a particular condition. “Treatment” may also reduce the severity of an existing condition.

The development of a method for generating MLPCs and MLPC-derived cells in vitro has now facilitated the development of in vitro based screening systems for testing the effectiveness and toxicity of existing or potential treatment or culture regimes.

Thus, according to yet another aspect of the present invention, there is provided a method of assessing the effect of a treatment or culture regime on the phenotypic or functional state of a MLPC or MLPC-derived cell said method comprising subjecting said MLPC or MLPC-derived cell, which cell has been generated in accordance with the method hereinbefore defined, to said treatment regime and screening for an altered functional or phenotypic state.

Preferably, said MLPC is a haematopoietic or mesenchymal stem cell.

By “altered” is meant that one or more of the functional or phenotypic parameters which are the subject of analysis are changed relative to untreated cells. This may be a desirable outcome where the treatment regime in issue is designed to improve cellular functioning. However, where the treatment regime is associated with a detrimental outcome, this may be indicative of toxicity and therefore the unsuitability for use of the treatment regime. It is now well known that the differences which are observed in terms of the responsiveness of an individual to a particular drug are often linked to the unique genetic makeup of that individual. Accordingly, the method of the present invention provides a valuable means of testing either an existing or a new treatment regime on cells which are generated utilising nuclear material derived from the individual in issue. This provides a unique means for evaluating the likely effectiveness of a drug on an individual's cellular system prior to administering the drug in vivo. Where a patient is extremely unwell, the physiological stress which can be caused by a treatment regime which causes an unwanted outcome can be avoided or at least minimised.

Accordingly, this aspect of the present invention provides a means of optimising a treatment which is designed to normalise cellular functioning. However the method can also be used to assess the toxicity of a treatment, in particular a treatment with a compound. Thus, failure to generate a characteristic associated with a haematopoietic or mesenchymal phenotype, for example, in the cells and tissues of the present invention in response to treatment with a compound can be used to assess the toxicity of such a compound.

Hence the method of the present invention can be used to screen and/or test drugs, other treatment regimes or culture conditions. In the context of assessing phenotypic changes, this aspect of the present invention can be utilized to monitor for changes to the gene expression profiles of the subject cells and tissues. Thus, the method according to this aspect of the present invention can be used to determine, for example, gene expression pattern changes in response to a treatment.

Preferably, the treatment to which the cells or tissues of the present invention are subjected is an exposure to a compound. Preferably, the compound is a drug or a physiological ion. Alternatively the compound can be a growth factor or differentiation factor. To this end, it is highly desirable to have available a method which is capable of predicting such side effects on cellular populations prior to administering the drug.

The present invention is further described by reference to the following non-limiting examples.

### Example 1

### CD Markers and Proteins Expression in CD4+-, CD8+-, CD19+-, CD20+- and CD25+-PBMC

### Cell Culture

Peripheral blood mononuclear cells (PBMCs) were collected from healthy volunteers aged 20-40 and fractioned by GE Ficoll-Paque PLUS (GE Healthcare Instructions 71-7167-00 AG) according to the the product instruction manual.

CD4+, CD8+, CD19+, CD20+ and CD25+ leukocytes were generated from PBMCs using a selected adherent method Briefly, these five populations of lymphocytes were individually purified from PBMCs by microbeads (MACS), the purities were routinely >90%, verified by flow cytometry.

Each population of these lymphocytes was cultured in sterile FEP culture bag individually. These final culture media were reconstituted of 30% of CD4⁺ and CD8⁺-PBMC, 40% of 6% human albumin (CSL Behring) solution and 30% of cell culture medium, and 2% insulin (Invitrogen, USA). 40% of CD19+-PBMC cells was reconstituted 20% of 6% human albumin (CSL Behring) solution and 40% of cell culture medium. 20% of CD20⁺ and CD25⁺ cells were reconstituted 40% of 6% human albumin (CSL Behring) solution and 40% of cell culture medium, and 2% insulin (Invitrogen, USA). Cells were grown in these mixtures for 3-6 days at 37° C. in a humidified incubator with 5% CO₂.

During this incubation period, the five lymphocyte populations (CD4⁺, CD8⁺, CD19⁺, CD20⁺ and CD25⁺) were examined for CD markers and surface proteins expression by flow cytometry. Furthermore, in CD4⁺ population, total cell protein expression were examined by Western blotting.

### Morphological Observation of PBMC

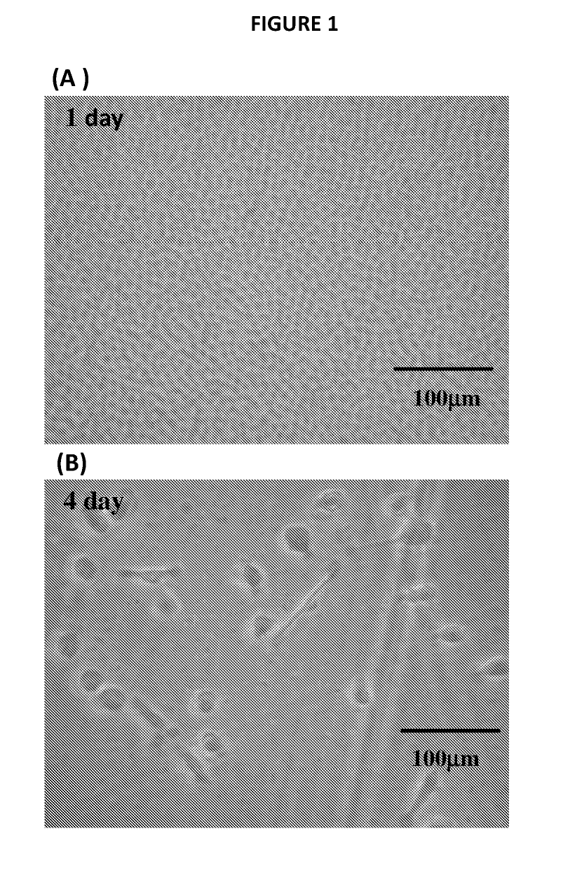

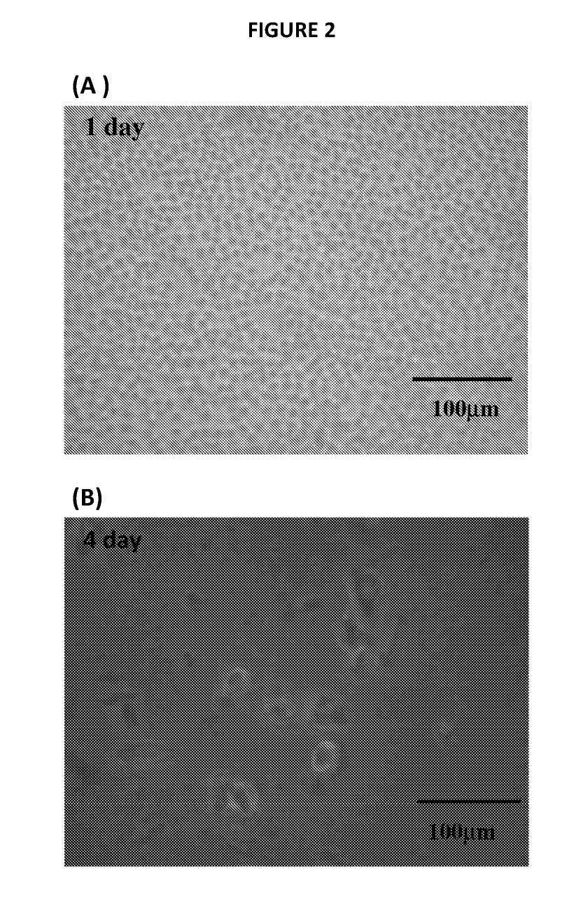

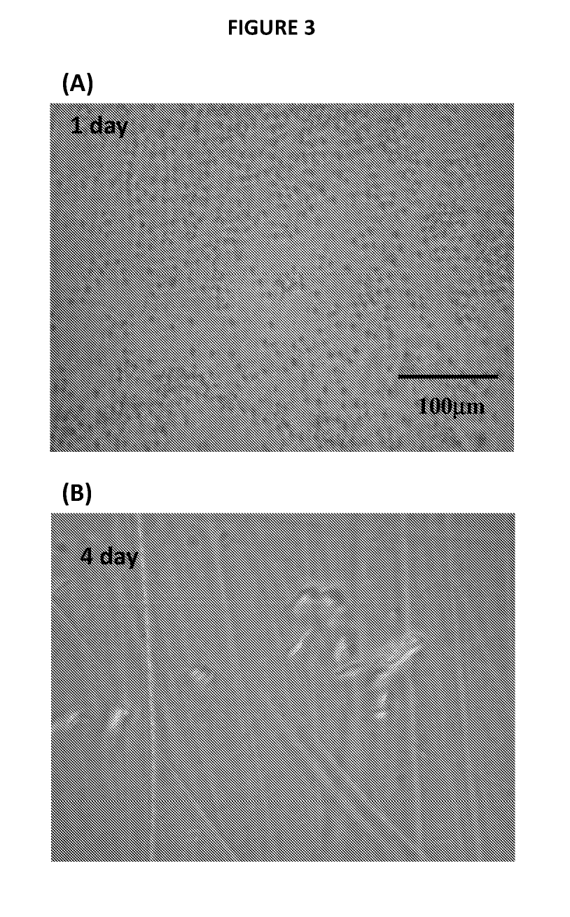

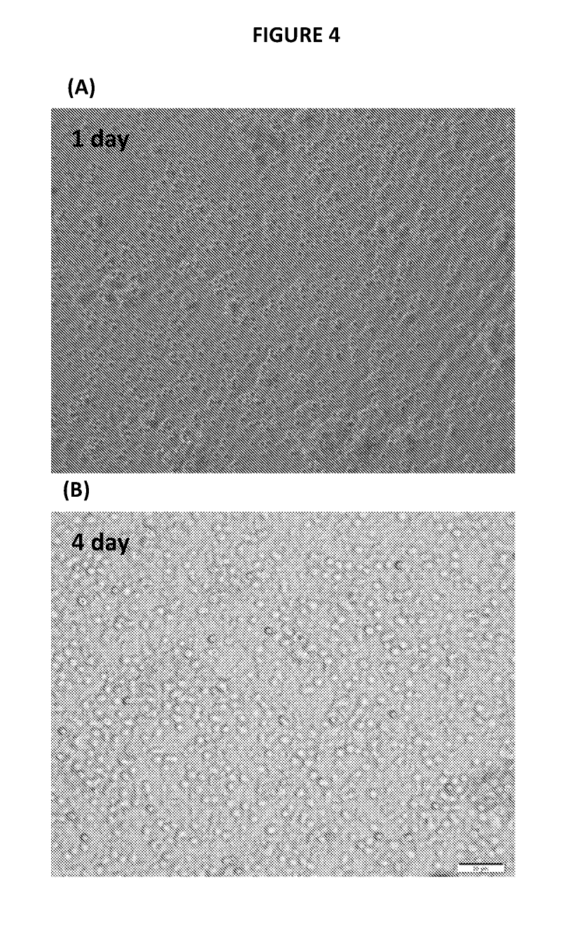

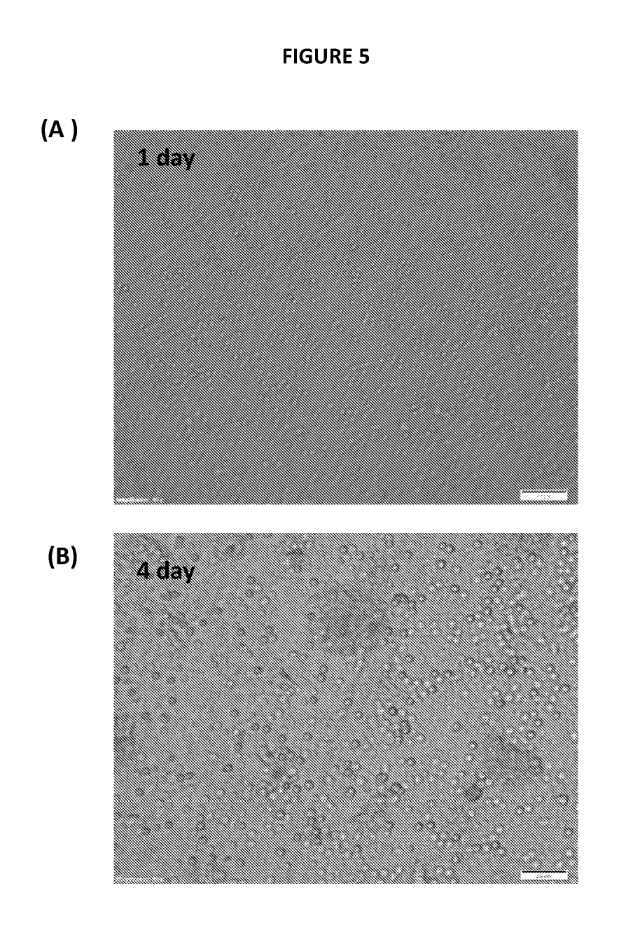

Slides were prepared with samples of the cell culture from 1 day, and 4 day post-incubation in a CO₂ incubator at 37° C. To study PBMC's biological characteristics, adherent cells phenotypes were analysed by an inverted microscope during cell cultivation periods (FIGS. 1 to 5).

CD Markers Expression of CD4+, CD8+, CD19+, CD20+- and CD25+ by Flow Cytometry Analysis

CD4⁺-, CD8⁺-, CD19⁺-, CD20⁺- and CD25⁺-PBMC were harvested respectively and washed with PBS (containing 2% Fetal Bovine Serum; FBS) from FEP culture bag, centrifuged at 640×g at 4° C. for 5 minutes, cell pellets were kept. The cell density was adjusted to 3×10⁵ cells per tube for flow cytometry assay. These five leucocyte populations were labelled with fluorescence-labelling antibodies. Finally, a 100 microliter fixation buffer (BD) was added to each tube and then incubated at 4° C. for 20 minutes, and finally stored in dark at 4° C. until flow cytometry analysis (Bacton Dickinson). Viable cells were identified by using the CellQuest software, and the resultant data are shown in Tables 1-10.

Proteins Expression of cD4⁻, CD8⁺, CD19⁺, CD20⁺ and CD25⁺ Lymphocytes by Western Blotting Analysis

### Preparation of Cells Extract

Cell proteins were extracted individually from CD4⁺-PBMC cells from 40 healthy volunteer after culturing for 3-6 days. Briefly, cell protein extraction was obtained by RIPA Lysis Buffer (Millipore, Temecula. Calif. 92590). The extracted suspension was incubated on ice for 20 min and then centrifuged at 13000×g for 5 min. The supernatant (the soluble fraction) was collected for proteins expression.

### Western Blot Analysis

Antibodies against various proteins were purchased from commercially available products. These include Collage Type I, HLA Class-1, TAZ, Insulin-like growth factor-binding protein 3 (IGFBP3), Alkaline Phosphatase, Nerve growth factor (NGF), Tumor necrosis factor ligand superfamily member 18 (TNFSF18), Stem cell antigen-1 (Sca-1), Caveolin-2, and Perforin (Abcam Inc.); CDX2, Fibronectin, Macrophage-1 antigen (MAC-1), M Cadherin, MyoD (MYOD1), Nuclear transcription factor Y subunit alpha (NF-YA), Notch 1, Paired box-5 (PAX-5), P− glycoprotein, Wiskott-Aldrich Syndrome Protein (WASP). (Epitomic Inc); α-Actinin, Ca2+/calmodulin-dependent protein kinase (CaM kinase IV), Cellular retinoic acid binding protein (CRABP II), GATA binding factor-4 (GATA4), Hypoxia-inducible factor-1a (HIF-1a), Myogenin, Achaete-scute homolog 1 (ASCL1), Synaptophysin (SYP), Nestin and Runt-related transcription factor 3 (Runx3) (Merck Millipore Headquarters.), Annexin VI (G-10), Neurogenin 3 (E-8), Granzyme B, Glutamate decarboxylase (GAD2, D5G2), Neuropilin-2, β Enolase (ENO-3), Granulocyte-colony stimulating factor (G-CSF) and Granulysin (F-9).(Santa Cruz Biotechnology.), Fms-related tyrosine kinase 1 (FLT-1) and Multidrug resistance-associated protein 1 (MRP1) (CHEMICON international, a division of SerlogicalR Corporation) and PU.1 (Cell Signaling Technology, Inc.)

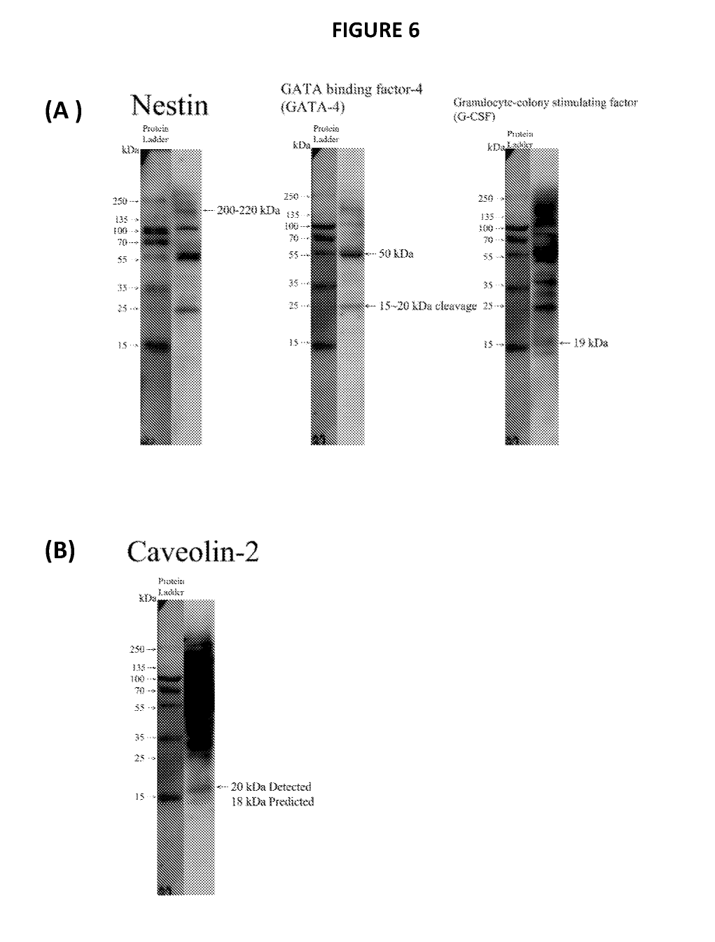

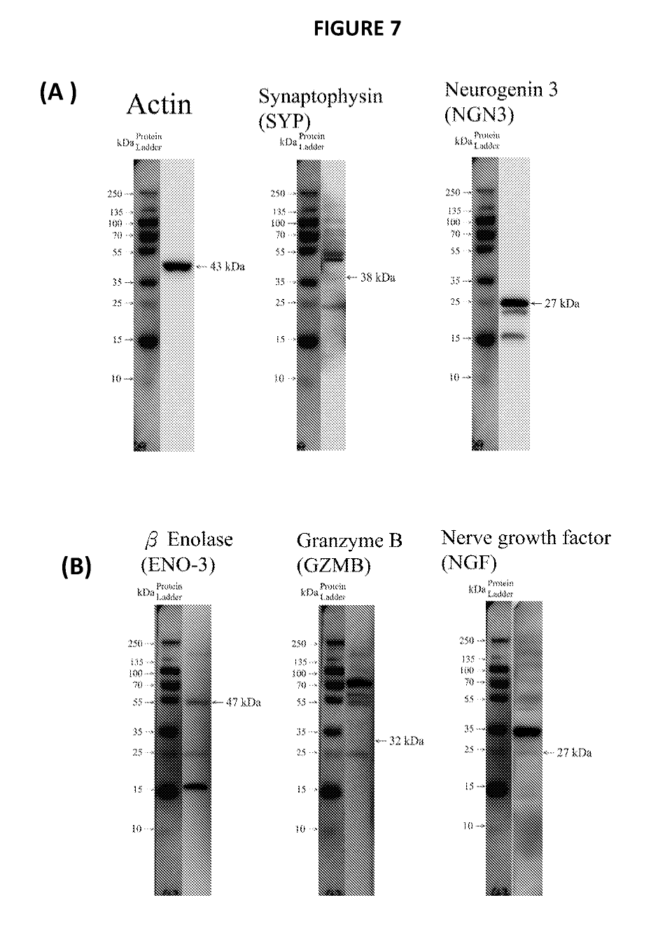

The supernatants of these cell lysates were used for sodium dodecyl sulfate-polyacrylamide gel electrophoresis analysis. One hundred micrograms of each cell lysate sample was loaded onto the Pierce 4-20% Tris-glycine Gel (Thermo SCIENTIFIC, Rockford USA). After electrophoresis, the gels were blotted onto PVDF membranes (Millpore, Temecula. Calif. 92590). The PVDF membranes were subjected to blocking with 5% skim milk in Tris-buffered saline Tween-20 buffer (10 mM Tris, pH 8.0, 150 mM NaCl) and the membranes were then incubated with the various primary antibodies in fresh 5% skim milk Tris-buffered saline Tween-20 buffer at 2-5° C. for 18-20 hours. The membranes were washed and then incubated with horseradish peroxidase-conjugated secondary antibody. Visualization of bands was performed with an Amersham-enhanced chemiluminescence system. Responsive bands were recorded by CCD camera and analyzed by Multi Gauge software. Semi-quantitative analysis of the percentage increase in expression was determined, using an internal control of beta-actin normalization. The results are presented here in FIGS. 6-7 and Table 11.

TABLE 1
Flow cytometric analysis of the multilineage progenitor cells derived
from CD4⁺ PBMCs which have been cultured according
to the method of the present invention
Proteins expression of CD4⁺ PBMCs by Flow Cytometry Analysis

| CD markers | Isotype | % of positive cells |
|---|---|---|
| αβTCR | m IgG1 | 99.52 |
| CLA | r IgM | 6.31 |
| EGFR | m IgG1 | 1.76 |
| HER-2 (c-new) | m IgG1 | 5.04 |
| HLA-A,B,C | m IgG2a | 99.91 |
| HLA-A2 | m IgG2b | 8.46 |
| HLA-DQ | m IgG1 | 3.09 |
| HLA-DR | m IgG2a | 10.69 |
| HLA-DR,DP,DQ | m IgG2a | 4.79 |
| Integrin-β7 | r IgG2b | 19.79 |
| MIC A/B | m IgG2a | 0.37 |
| MHC Class I free chain | m IgG1 | 0.02 |
| without beta2 microglobulin | | |
| SSEA-1 | m IgM | 0.17 |
| SSEA-3 | r IgM | 1.12 |
| SSEA-4 | m IgG3 | 0.85 |
| TRA-1-60 | m IgM | 1.22 |
| TRA-1-81 | m IgM | 2.80 |
| Vβ8 | m IgG2b | 0.12 |
| Vβ23 | m IgG1 | 5.48 |

Total 19
Positive 14

TABLE 2
Flow cytometric analysis of the multilineage progenitor cells derived from CD4⁺ PBMCs
which have been cultured according to the method of the present invention
CD markers expression of CD4⁺ PBMCs by Flow Cytometry Analysis

| CD markers | Alternate names | Isotype | % of positive cells |
|---|---|---|---|
| CD1a | R4, T6, Leu-6, HTA1 | m IgG1 | 1.70 |
| CD1b | R1, T6 | m IgG1 | 0.01 |
| CD1c | BDCA-1, R7, T6, M241 | m IgG1 | 0.15 |
| CD1d | R3, R3G1 | m IgG2b | 0.33 |
| CD2 | T11, LFA-2, SRBC-R, E-rosette R, Erythrocyte R | m IgG1 | 99.91 |
| CD3 | T3 | m IgG1 | 99.92 |
| CD4 | T4, Leu-3, L3T4, Leu-3a, W3/25 | m IgG1 | 99.51 |
| CD5 | T1, Tp67, Leu-1, Ly-1 | m IgG1 | 99.83 |
| CD6 | T12, TP120 | m IgG1 | 99.93 |
| CD7 | gp40, Leu-9, TP41 | m IgG2a | 96.18 |
| CD8 | T8, Leu-2 | m IgG1 | 1.01 |
| CD8a | type I glycoprotein | m IgG1 | 4.02 |
| CD8b | Lyt3 | m IgG1 | 0.24 |
| CD9 | p24, MRP-1, DRAP-27, DRAP-1 | m IgG1 | 56.29 |
| CD10 | CALLA, NEP, gp100, EC 3.4.24.11, MME | m IgG2b | 1.27 |
| CD11a | LFA-1, integrin αL, ITGAL, LFA-1α | m IgG1 | 88.55 |
| CD11b | Mac-1, integrin αM, CR3, ITGAM, Mo1, C3niR | m IgG1 | 4.93 |
| CD11c | p150, 95, CR4, integrin αX, ITGAX, AXb2 | m IgG1 | 2.24 |
| CD13 | APN, gp150, Amniopeptidase N, ANPEP, AAP, | m IgG1 | 3.64 |
| | APM, LAP1, P150, PEPN, EC 3.4.11.2 | | |
| CD14 | LPS-Receptor | m IgG1 | 2.54 |
| CD15 | Lewis X, Lex, SSEA-1, 3-FAL, X-Hapten, FUT4 | m IgM | 3.13 |
| CD15s | Sialyl Lewis X | m IgM | 0.82 |
| CD16 | FCRIIIA, CD16a | m IgG1 | 2.91 |
| CD16b | FCRIIIB, FcγRIIIB | m IgG2a | 1.81 |
| CD17 | Lactosylceramide, LacCer | m IgG1 | 2.38 |
| CD18 | Integrin β2, ITGB2, CD11a, b, c β-subunit | m IgG1 | 99.86 |
| CD19 | B4 | m IgG1 | 1.37 |
| CD20 | B1, Bp35, Ly-44 | m IgG2b | 2.58 |
| CD21 | CR2, EBV-R, C3dR | m IgG2a | 10.93 |
| CD22 | BL-CAM, Siglec-2 | m IgG1 | 1.36 |
| CD23 | FcεRII, BLAST-2, FceRII, B6, Leu-20 | m IgG1 | 0.91 |
| CD24 | BA-1, HAS, HSA, BBA-1 | m IgG1 | 3.79 |
| CD25 | p55, IL-2Rα, Tac antigen, Tac, TCGFR | m IgG1 | 11.54 |
| CD26 | DPP IV ectoenzyme, DPP IV, ADA binding | m IgG1 | 93.77 |
| | protein, ADCP2, TP103 | | |
| CD27 | T14, S152, TNFRSF7, TP55 | m IgG1 | 93.17 |
| CD28 | Tp44, T44 | m IgG1 | 99.73 |
| CD29 | Integrin β1, platelet GPIIa, ITGB1, GP | m IgG1 | 99.95 |
| CD30 | Ki-1, Ber-H2, TNFRSF8 | m IgG1 | 1.58 |
| CD31 | PECAM-1, endocam, GPIIa, Platelet | m IgG1 | 47.09 |
| | endothelial cell adhesion molecule, PECA1 | | |
| CD32 | FcγRII | m IgG1 | 0.49 |
| CD33 | p67, Siglec-3, My9, gp67, Sialic acid-binding | m IgG1 | 4.44 |
| | Ig-like lectin 3, Myeloid cell surface antigen | | |
| | CD33 | | |
| CD34 | gp105-120, Mucosialin, My10, Hematopoietic | m IgG1 | 4.39 |
| | progenitor cell antigen 1 (HPCA1) | | |
| CD35 | CR1, C3b/C4b receptor, Immune adherence | m IgG1 | 3.85 |
| | receptor, Complement receptor 1 | | |
| CD36 | GPIV, OKM5 antigen, PASIV, Glycoprotein IIIb | m IgM | 25.12 |
| | (GpIIIb), Glycoprotein IV (GPIV), Fatty acid | | |
| | translocase (FAT), SCARB3, GP88, Platelet | | |
| | glycoprotein 4 | | |
| CD37 | gp 52-40, Tspan-26, Leukocyte antigen CD37, | m IgG1 | 0.02 |
| | Tetraspanin-26, TSPAN26 | | |
| CD38 | T10, ADP-ribosyl cyclase, Cyclic ADP-ribose | m IgG1 | 36.69 |
| | hydrolase 1 | | |
| CD39 | NTPDase-1, gp80, EC3.6.1.5, Ectonucleoside | m IgG1 | 8.39 |
| | triphosphate diphosphohydrolase 1 | | |
| | (ENTPD1), ATPdehydrogenase | | |
| CD40 | Bp50, TNFRSF5, MGC9013, Tumor necrosis | m IgG1 | 2.61 |
| | factor receptor superfamily member 5 | | |
| CD41 | ITGA2B, GPIIb, Integrin αIIb, Platelet | m IgG1 | 12.54 |
| | membrane glycoprotein IIb, Integrin α2b, | | |
| | Human Platelet Antigen-3 | | |
| | (HPA-3) | | |
| CD41a | Integrin alpha Iib, platelet GPIIb | m IgG1 | 7.54 |
| CD41b | fibrinogen receptor, gpIIb/IIIa, integrin alpha | m IgG3 | 37.67 |
| | IIb, ITGA2b | | |
| CD42a | GPIX, GP9, Platelet glycoprotein IX | m IgG1 | 6.64 |
| CD42b | gpIbα, GPIba, Platelet glycoprotein Ib α | m IgG1 | 4.61 |
| CD42d | Glycoprotein V, GPV, Platelet glycoprotein V | m IgG1 | 5.07 |
| CD43 | gpL115, Sialophorin, Leukosialin, | mIgG1 | 99.34 |
| | Galactoglycoprotein, SPN | | |
| CD44 | H-CAM, Pgp-1, EMCR III, CD44s, Hermes | m IgG2b | 99.64 |
| | antigen, ECMRII, Phagocytic glycoprotein I, | | |
| | Extracellular matrix receptor III, GP90 | | |
| | Lymphocyte homing/adhesion receptor, | | |
| | Hyaluronate receptor | | |
| CD45 | Leukocyte Common Antigen (LCA), T200, | m IgG1 | 99.81 |
| | B220, Ly5, Protein tyrosine phosphatase | | |
| | receptor type C (PTPRC) | | |
| CD45RA | PTPRC | m IgG2b | 82.83 |
| CD45RB | PTPRC | m IgG2b | 99.91 |
| CD45RO | UCHL-1 | m IgG2a | 83.12 |
| CD46 | Membrane Cofactor Protein (MCP), | m IgG1 | 99.90 |
| | Trophoblast leukocyte common antigen, | | |
| | TRA2.10 | | |
| CD47 | IAP, neurophilin, gp42, OA3, MER6 | m IgG1 | 99.94 |
| CD48 | Blast-1, BCM1, Sgp-60, SLAMF2, Hulym3, OX- | m IgG1 | 99.74 |
| | 45, MEM-102 | | |
| CD49a | VLA-1α, Integrin α1, VLA-1, ITGA1 | m IgG1 | 17.85 |
| CD49b | VLA-2α, gpIa, Integrin α2, VLA-2, ITGA2 | m IgG1 | 70.90 |
| CD49c | VLA-3α, Integrin α3, VLA-3, ITGA3, GAPB3, | m IgG1 | 0.04 |
| | Galactoprotein B3, MSK18, Very Common | | |
| | Antigen-2 (VCA-2) | | |
| CD49d | VLA-4α, Integrin α4, VLA-4, ITGA4 | m IgG1 | 94.25 |
| CD49e | VLA-5α, Integrin α5, VLA-5, ITGA5, | m IgG3 | 62.51 |
| | Fibronectin receptor | | |
| CD49f | VLA-6α, Integrin α6, VLA-6, ITGA6, gpI | r IgG2b | 29.16 |
| CD50 | ICAM-3 | m IgG1 | 99.96 |
| CD51/61 | vitronectin R, Integrin αv, VNR-α, Vitronectin- | m IgG1 | 4.18 |
| | Rα, ITGAV, Integrin αvβ3 | | |
| CD52 | CAMPATH-1, HE5, Epididymal secretory | m IgG2b | 99.77 |
| | protein E52, HES | | |
| CD53 | OX-44, MCR, TSPAN25, MOX44, Tetraspanin- | m IgG1 | 99.91 |
| | 25 | | |
| CD54 | ICAM-1 | m IgG1 | 73.13 |
| CD55 | Decay Accelerating Factor for Complement | m IgG1 | 99.28 |
| | (DAF) | | |
| CD56 | Leu-19, NKH-1, Neural Cell Adhesion | m IgG1 | 53.78 |
| | Molecule (NCAM) | | |
| CD57 | HNK-1, Leu-7, β-1,3-glucuronyltransferase 1, | m IgM | 28.19 |
| | Glucuronosyltransferase P, | | |
| | galactosylgalactosylxylosyl protein 3-β- | | |
| | glucuronosyltransferase 1 | | |
| CD58 | LFA-3 | m IgG1 | 0.01 |
| CD59 | Protectin, H19, 1F-5Ag, MIRL, MACIF, P-18 | m IgG1 | 99.94 |
| CD60b | 9-O-sialyl GD3 | m IgM | 32.03 |
| CD61 | GP IIIa, Integrin β3 | m IgG1 | 7.41 |
| CD62E | E-selectin, ELAM-1, LECAM-2 | m IgG1 | 4.72 |
| CD62L | L-selectin, LECAM-1, LAM-1, Leu-8, TQ1, MEL- | m IgG1 | 88.47 |
| | 14 | | |
| CD62P | P-selectin, GMP-140, PADGEM | m IgG1 | 7.81 |
| CD63 | LIMP, MLA1, LAMP-3, ME491, gp55, NGA, | m IgG1 | 73.81 |
| | OMA81H, TSPAN30, Granulophysin, | | |
| | Melanoma 1 antigen | | |
| CD64 | FcγRI, FcRI | m IgG1 | 3.06 |
| CD65 | Ceramide-dodecasaccharide, VIM2, | m IgM | 1.96 |
| | Fucoganglioside (Type II) | | |
| CD65s | Sialylated poly-N-acetyllactosamine, | m IgM | 9.88 |
| | Sialylated-CD65, VIM2 | | |
| CD66 | | m IgG2a | 9.70 |
| CD66a | NCA-160, BGP (Biliary glcoprotein), BGP1, | m IgG2a | 0.00 |
| | BGPI, CEACAM1 | | |
| CD66b | CD67, CGM6, NCA-95, CEACAM8 | m IgM | 1.00 |
| CD66c | NCA, NCA-50/90, CEAL, CEACAM6 | m IgG1 | 4.88 |
| CD68 | gp110, Macrosialin, SCARD1 | m IgG2b | 2.92 |
| CD69 | AIM, VEA, MLR3, EA 1, gp34/28, CLEC2C, BL- | m IgG1 | 3.61 |
| | AP26 | | |
| CD70 | Ki-24, CD27L, TNFSF7, CD27LG | m IgG1 | 6.42 |
| CD71 | TfR, T9, TFRC, Transferrin receptor, TRFR | m IgG1 | 22.62 |
| CD72 | Lyb-2, Ly-32.2, Ly-19.2 | m IgG2b | 8.35 |
| CD73 | NT5E, Ecto-5′-nuclotidase, E5NT, NT5, NTE, | m IgG1 | 21.10 |
| | eN, eNT | | |
| CD74 | Ii, invariant chain, DHLAG, HLADG, Ia-γ | m IgG1 | 42.68 |
| CD75 | lactosamines, ST6GAL1, MGC48859, SIAT1, | m IgM | 1.60 |
| | ST6GALL, ST6N, ST6 β-Galactosamide α-2,6- | | |
| | sialyltranferase, | | |
| | Sialo-masked lactosamine, Carbohydrate of | | |
| | α2,6 sialyltransferase | | |
| CD77 | Pk Ag, BLA, CTH, Gb3, Pk blood groupBLA, | m IgM | 21.31 |
| | A14GALT (α1,4-Galactosyltransferase), | | |
| | A4GALT1, Gb3S, P(k), P1, PK A4GALT, Pk | | |
| | antigen, CTH/Gb3A4GALT1, Gb3S, PK, | | |
| | P1 | | |
| CD79a | Igα, MB1, IGA (Immunoglobulin-associated a), | m IgG1 | 4.11 |
| | MB-1 | | |
| CD79b | B29, Igβ (Immunoglobulin-associated β) | m IgG1 | 5.10 |
| CD80 | B7, B7-1, BB1, CD28LG, CD28LG1, L AB7 | m IgG1 | 2.33 |
| CD81 | TAPA-1, S5.7 | m IgG1 | 1.63 |
| CD83 | HB15, BL11 | m IgG1 | 2.16 |
| CD84 | GR6, SLAMF5, LY9B, p75, hly9-β | m IgG1 | 0.01 |
| CD85a | ILT5, LIR3, HL9, LILRB3 (Leukocyte | r IgG2b | 3.84 |
| | immunoglobulin-like receptor, subfamily B | | |
| | (with TM and ITIM domains), member 3, LIR- | | |
| | 3, MGC138403, PIRB, XXbac-BCX105G6.7 | | |
| CD85d | ILT4, LILRB2 (Leukocyte immunoglobulin-like | r IgG2b | 9.18 |
| | receptor, subfamily B (with TM and ITIM | | |
| | domains), member 2, LIR2, MIR10, MIR-10 | | |
| CD85f | LIT11, LILRA5, XXbac-BCX403H19.2, LIR9, | m IgG2a | 5.76 |
| | LILRB7 (Leukocyte immunoglobulin-like | | |
| | receptor, subfamily B (with TM and ITIM | | |
| | domains), member 7 | | |
| CD85g | ILT7, LILRA4 (Leukocyte immunoglobulin-like | m IgG1 | 1.27 |
| | receptor, subfamily A (with TM domain), | | |
| | member 4, MGC129597, MGC129598, LIR4 | | |
| CD85h | ILT1, LILRA2 (Leukocyte immunoglobulin-like | r IgG2b | 3.95 |
| | receptor, subfamily A (with TM domain), | | |
| | member 2, LIR7, LIR-7, XXbac-BCX85G21.2, | | |
| | ILT-1 | | |
| CD85i | LILRA1 (Leukocyte immunoglobulin-like | m IgG2b | 0.01 |
| | receptor), subfamily A (with TM domain), | | |
| | member 1, LIR6, LIR-6, MGC126563 | | |
| CD85j | ILT2, LILRB1 (Leukocyte immunoglobulin-like | m IgG1 | 2.89 |
| | receptor, subfamily B (with TM and ITIM | | |
| | domains), member 1, FLJ37515, LIR-1, LIR1, | | |
| | MIR-7, MIR7 | | |
| CD85k | ILT3, LILRB4 (Leukocyte immunoglobulin-like | m IgG1 | 2.28 |
| | receptor, subfamily B (with TM and ITIM | | |
| | domains), member 4, LIR-5, HM18, LIR5, | | |
| | LILRB5 | | |
| CD86 | B70, B7-2, CD28LG2, LAB72, MGC34413 | m IgG2b | 4.06 |
| CD87 | UPA-R, PLAUR, URKR | m IgG1 | 0.82 |
| CD88 | C5aR, C5aR C5R1, C5R1, C5AR, C5A | m IgG2a | 6.37 |
| CD89 | FcaR, IgA R | m IgG1 | 5.33 |
| CD90 | Thy-1 | m IgG1 | 2.70 |
| CD91 | α2M-R, LRP, LRP1, α2MR, APOER, APR | m IgG1 | 5.53 |
| CD92 | SLC44A1, CTL1, CHTL1, RP11-287A8.1, p70, | m IgG2b | 40.85 |
| | CDw92 | | |
| CD93 | C1qRp, C1QR1, C1qRP, MXRA4, C1qR(P), | m IgM | 9.60 |
| | Dj737e23.1, GR11 | | |
| CD94 | Kp43, KLRD1 | m IgG1 | 3.88 |
| CD95 | Fas, APO-1, TNFRSF6, CD178, FASLG, CD95L, | m IgG1 | 83.03 |
| | APT1LG1, APT1, FAS1, FASTM, ALPS1A, | | |
| | TNFSF6, FASL | | |
| CD96 | TACTILE, MGC22596 | m IgG1 | 75.47 |
| CD97 | EMR1, BL-KDD/F12, TM&LN1 | m IgG1 | 0.01 |
| CD98 | 4F2, FRP-1, RL-388, SLC3A2, 4F2HC, 4T2HC, | m IgG1 | 8.53 |
| | MDU1, NACAE | | |
| CD99 | MIC2, E2, MIC2, MIC2X, MIC2Y, HBA71, | m IgG2a | 99.82 |
| | MSK5X | | |
| CD99R | E2, CD99 Mab restricted | m IgM | 99.39 |
| CD100 | SEMA4D, SEMAJ, coll-4, C9orf164, FLJ33485, | m IgM | 98.23 |
| | FLJ34282, FLJ39737, FLJ46484, M-sema-G, | | |
| | MGC169138, MGC169141, SEMAJ | | |
| CD101 | BB27, V7, P126, IGSF2, BA27, BPC#4, V7-LSB | m IgG1 | 0.00 |
| CD102 | ICAM-2, Ly60 | m IgG2a | 99.71 |
| CD103 | HML-1, Integrin αE, aIEL, ITGAE, OX62, HML1 | m IgG1 | 1.19 |
| CD104 | TSP-180, lntegrin β4, TSP1180, ITGB4 | r IgG2b | 0.04 |
| CD105 | Endoglin, ENG, HHT1, ORW, SH-2 | m IgG1 | 9.04 |
| CD106 | VCAM-1, INCAM-110, V-CAM, INCAM-100 | m IgG1 | 1.60 |
| CD107a | LAMP-1, LAMPA, CD107a, LGP120 | m IgG1 | 88.12 |
| CD107b | LAMP-2, LAMPB | m IgG1 | 4.22 |
| CD108 | SEMA7A, JMH blood group antigen, JMH | m IgM | 68.23 |
| CD109 | 8A3, 7D1, E123, Platelet activation factor, | m IgG1 | 9.91 |
| | 8As, 150 kD TGF-β-1-binding protein, Platelet- | | |
| | specific Gov antigen | | |
| CD110 | MPL, TPO-R, C-MPL | m IgG2a | 6.13 |
| CD111 | PRR1, Nectin-1, PVRL1, HveC, HIgR, CLPED1, | m IgG1 | 26.05 |
| | Hve C1 | | |
| CD112 | PRR2, Nectin-2, HveB, PVRL2 | m IgG1 | 16.47 |
| CD114 | G-CSFR, CSF3R, HG-CSFR | m IgG1 | 0.03 |
| CD115 | CSF-1R, M-CSFR, c-fms, FMS, FIM2 | r IgG1 | 11.01 |
| CD116 | GM-CSFRα, GM-CSFRa, CDw116, CSF2R, | m IgG1 | 81.12 |
| | CSF2RAX, CSF2RAY, CSF2RX, CSF2RY, GM-CSF- | | |
| | R-α, GMCSFR, GMR, MGC3848, MGC4838 | | |
| CD117 | c-kit, SCFR, PBT | m IgG1 | 5.06 |
| CD118 | LIFR, gp190, SJS2, STWS, SWS | m IgG1 | 18.08 |
| CD119 | IFNγR, IFNγRα, CDw119, IFNGR1, IFNγRa | m IgG1 | 48.71 |
| CD120a | TNFR-I, p55, TNFRSF1A, CD120a, FPF, | m IgG1 | 2.20 |
| | MGC19588, TBP1, TNF-R, TNF-R55, TNFAR, | | |
| | TNFR1, TNFR55, TNFR60, p55-R, p60 | | |
| CD120b | TNFR-II, p80, TNFRSF1B, p75, TNFR p80 | r IgG2b | 63.77 |
| CD121a | IL-1R type 1, IL-1RI, IL1R, CD121A, D2S1473, IL- | m IgG1 | 14.38 |
| | 1R-α, IL1RA, P80 | | |
| CD121b | IL-1R type II, IL-1RII | m IgG1 | 5.27 |
| CD122 | IL-2Rβ, IL2RB, p70-75 | m IgG2a | 19.88 |
| CD123 | IL-3Rα, IL3RA, CD123, IL3R, IL3RAY, IL3RX, | m IgG1 | 1.72 |
| | IL3RY, MGC34174, hIL-3Ra | | |
| CD124 | IL-4Rα, IL4R | m IgG2a | 2.69 |
| CD125 | IL-5Rα, CDw125, IL5RA | m IgG1 | 59.85 |
| CD126 | IL-6Rα, IL6R | m IgG1 | 0.01 |
| CD127 | IL-7R, IL-7Rα, IL7R, p90 | m IgG1 | 99.38 |
| CD129 | IL-9R, IL-9Rα | m IgG2b | 11.50 |
| CD130 | gp130, IL-6Rβ, IL6ST, IL6ST, IL6-β | m IgG1 | 86.78 |
| CD131 | CSF2RB, IL3RB, IL5RB, CDw131, IL-3Rβ, | m IgG1 | 3.49 |
| | common β chain, IL-3R common β | | |
| CD132 | Common γ chain, IL-2Rγ, IL2RG | r IgG2b | 84.65 |
| CD133 | AC133, PROML1, Prominin 1, Hematopoietic | m IgG1 | 1.89 |
| | stem cell antigen, prominin-like 1 | | |
| CD134 | OX-40, TNFRSF4 | m IgG1 | 62.90 |
| CD135 | Flt3/Flk2, STK-1 | m IgG1 | 3.19 |
| CD136 | MSP-R, RON, p158-ron, CDw136, MST1R | m IgG1 | 89.81 |
| CD137 | 4-1BB, TNFRSF9, ILA | m IgG1 | 4.01 |
| CD137 | 4-1BB Ligand | m IgG1 | 12.90 |
| Ligand | | | |
| CD138 | Syndecan-1, Heparan sulfate proteoglycan | m IgG1 | 79.06 |
| CD140a | PDGFRA, PDGF α Receptor, PDGFRα | m IgG1 | 22.27 |
| CD140b | PDGFRB, PDGF β Receptor, PDGFRβ | m IgG1 | 41.19 |
| CD141 | Thrombomodulin, THBD, Fetomodulin | m IgG1 | 17.47 |
| CD142 | Tissue Factor (TF), Factor III, Thromboplastin | m IgG1 | 2.50 |
| CD144 | VE-Cadherin, Cadherin-5 | m IgG1 | 10.83 |
| CD146 | MUC18, S-endo, MCAM, Mel-CAM, Endo- | m IgG1 | 6.87 |
| | CAM | | |
| CD147 | Neurothelin, basigin, EMMPRIN, BSG, M6, | m IgG1 | 1.53 |
| | OX47, TCSF | | |
| CD148 | HPTP-eta, p260, DEP-1, HPTP-η, SCC1, PTPRJ | m IgG1 | 33.46 |
| CD150 | SLAM, IPO-3 | m IgG1 | 44.28 |
| CD151 | PETA-3, Tspan-24, RAPH, SFA-1 | m IgG1 | 0.05 |
| CD152 | CTLA-4 | m IgG2a | 0.38 |
| CD153 | CD30L, TNFSF8, TNSF8 | m IgG2b | 71.41 |
| CD154 | CD40L, T-BAM, gp39, TRAP, TNFSF5, TRAP-1, | m IgG1 | 37.01 |
| | IMD3 | | |
| CD155 | PVR, Necl-5, PVS, TAGE4, HVED, NECL5 | m IgG2a | 3.40 |
| CD156b | TACE, ADAM17, cSVP | m IgG1 | 10.18 |
| CD156c | ADAM10, MADM, kuz | m IgG2b | 99.69 |
| CD157 | BST-1, Bp3, Mo5 | m IgG1 | 4.35 |
| CD158a | KIR2DL1, p58.1, NKAT1 | m IgG2b | 3.32 |
| CD158b | p58.2, KIR2DL2/L3, NKAT2 | m IgG2a | 5.72 |
| CD158d | KIR2DL4, KIR103A5, KIR103 | m IgG1 | 9.56 |
| CD158e1 | KIR3DL1, NKB1, NKB1B, p70 | m IgG1 | 1.06 |
| CD158f | KIR2DL5A, KIR2DL5 | m IgG1 | 1.64 |
| CD159a | NKG2A, KLRC1 | m IgG2a | 1.34 |
| CD159c | NKG2C, KLRC2 | m IgG1 | 0.57 |
| CD160 | BY55, NK1, NK28 | m IgM | 13.76 |
| CD161 | NKR-P1A, KLRB1, NKR | m IgG1 | 41.88 |
| CD162 | PSGL-1 | m IgG2a | 99.75 |
| CD163 | M130, GHI/61, D11, RM3/1 | m IgG1 | 1.11 |
| CD164 | MGC-24, MUC-24, Endolyn | m IgG2a | 92.03 |
| CD165 | AD2, gp37 | m IgG1 | 72.50 |
| CD166 | ALCAM, KG-CAM, SC-1, BEN, DM-GRASP | m IgG1 | 9.67 |
| CD167a | DDR1, trkE, cak | m IgG3 | 50.72 |
| CD169 | Sialoadhesin, Siglec-1 | m IgG1 | 0.78 |
| CD170 | Siglec-5, CD33-like2 | m IgG1 | 2.34 |
| CD171 | L1CAM, N-CAM L1, L1 antigen, HSAS, HSAS1, | m IgG2a | 3.57 |
| | MASA, MIC5, S10, SPG1, NILE | | |
| CD172a | SIRP alpha, BIT, MFR, MYD-1, P84, SHPS-1, | m IgG2a | 0.00 |
| | SHPS1, SIRPα2, SIRPa | | |
| CD172b | SIRPβ, SIRPβ1 | m IgG1 | 4.74 |
| CD172g | SIRPγ, SIRPβ2, SIRPγ, SIRP-B2, bA77C3.1 | m IgG1 | 95.60 |
| CD177 | NB1, HNA-2a, NB1gp, Neutrophil-specific | m IgG1 | 1.61 |
| | antigen 1, PRV1 | | |
| CD178 | CD95L, TNFSF6, Fas Ligand, FasL, APT1LG1 | m IgG1 | 1.07 |
| CD179a | VpreB, IGVPB, VPREB1 | m IgG1 | 17.29 |
| CD179b | Igλ5, λ5, 14.1, IGL5, IGGL1, IGO, lambda5 | m IgG1 | 8.14 |
| CD180 | RP105, LY64, Bgp95, Ly78 | m IgG1 | 4.55 |
| CD181 | CDw128A, IL-8RA, (formerly CD128a) CXCR1, | m IgG2b | 8.28 |
| | IL-8Rα | | |
| CD182 | CDw128B, IL-8RB, (formerly CD128b) CXCR2, | m IgG1 | 9.25 |
| | IL-8Rβ, CMKAR2, IL8R2 | | |
| CD183 | CXCR3, GPR9, CKR-L2, CMKAR3, IP10, Mig-R, | m IgG1 | 96.48 |
| | TAC | | |
| CD184 | CXCR4, Fusin, LESTR, NPY3R, CMKAR4, HM89, | m IgG2a | 99.18 |
| | FB22, LCR1 | | |
| CD185 | CXCR5, BLR1, MDR15, MGC117347 | m IgG2b | 34.21 |
| CD186 | CXCR6, CDw186, STRL33, TYMSTR, BONZO | m IgG2b | 6.06 |
| CD191 | CCR1, CKR1, CKR-1, HM145, CMKBR1, | m IgG2b | 0.06 |
| | SCYAR1, MIP-1αR, RANTES-R | | |
| CD192 | CCR2, CKR2, CCR2A, CCR2B, CKR2A, CKR2B, | m IgG2a | 28.99 |
| | CMKBR2, MCP-1-R, CC-CKR-2, FLJ78302, | | |
| | MGC103828, MGC111760, MGC168006 | | |
| CD193 | CCR3, CKR3, CMKBR3, CC-CKR-3, MGC102841 | m IgG2b | 3.17 |
| CD194 | CCR4, CC-CKR-4, CKR4, CMKBR4, ChemR13, | m IgG2b | 10.26 |
| | HGCN | | |
| CD195 | CCR5, CMKBR5, IDDM22, CC-CKR-5, FLJ78003 | m IgG1 | 7.43 |
| CD196 | CCR6, LARC receptor, DRY6, BN-1, DCR2, | m IgG1 | 11.07 |
| | CKRL3, GPR29, CKR-L3, CMKBR6, GPRCY4, | | |
| | STRL22, CC-CKR-6 | | |
| CD197 | EBI-1, BLR-2, CMKBR7, CCR7 (formerly | r IgG2a | 82.50 |
| | CDw197) | | |
| CD198 | CCR8, CKR-L1, CKRL1, CMKBR8, CMKBRL2, | r IgG2b | 5.51 |
| | CY6, GPR-CY6, TER1 | | |
| CD199 | CCR9, GPR28, GPR-9-6 | m IgG2a | 19.23 |
| CD200 | OX2, MRC, MOX1, MOX2 | m IgG1 | 30.21 |
| CD201 | EPC-R, PROCR, CCCA, CCD41, MGC23024, | m IgG1 | 0.01 |
| | bA42O4.2 | | |
| CD202b | Tie2 (Tek), TEK, VMCM, TIE-2, VMCM1 | m IgG1 | 10.88 |
| CD203c | E-NPP3, PD-1b, PDNP3, B10, PDIβ | m IgG1 | 0.84 |
| CD204 | MSR, MSR1, SR-A, phSR1, phSR2, SCARA1 | m IgG2b | 4.85 |
| CD205 | DEC-205, CLEC13B, GP200-MR6, LY75 | m IgG2b | 3.24 |
| CD206 | Mannose receptor C type-1 (MRC1), | m IgG1 | 5.22 |
| | Macrophage mannose receptor (MMR), C- | | |
| | type Lectin domain family 13 member D | | |
| | (CLEC13D) | | |
| CD207 | Langerin, C-type Lectin domain family 4 | m IgG1 | 4.81 |
| | member K (CLEC4K) | | |
| CD208 | DC-LAMP, Lysosomal-associated membrane | m IgG2a | 0.41 |
| | protein 3 (LAMP3), DCLAMP, LAMP, TSC403 | | |
| CD209 | Dendritic cell-specific ICAM-3-grabbing non- | r IgG2a | 5.72 |
| | integrin (DC-SIGN), DC-SIGN1, CDSIGN, C-type | | |
| | lectin domain family 4 member L (CLEC4L), | | |
| | HIV gp120-binding protein | | |
| CD210 | IL-10R | r IgG2a | 30.03 |
| CD210a | Interleukin 10 Receptor A (IL-10RA, IL-10R1), | m IgG1 | 8.64 |
| | IL-10Rα | | |
| CD210b | CRF2-4, Interleukin 10 Receptor B (IL-10RB, IL- | m IgG1 | 9.70 |
| | 10R2), IL-10Rβ, D21S58, CRFB4 | | |
| CD212 | IL-12Rβ1, IL12RB1, IL-12Rb1, Interleukin 12 | m IgG1 | 18.61 |
| | receptor β1 chain (IL-12β1), IL-12β, CD212b1 | | |
| CD213a1 | Interleukin 13 receptor α1 chain (IL-13Rα1), | m IgG2b | 4.63 |
| | NR4 | | |
| CD213a2 | IL12Rα2, IL-13Ra2, Interleukin 13 receptor α2 | m IgG1 | 10.68 |
| | chain (IL-13Rα2), interleukin-13-binding | | |
| | protein (IL13BP), IL13RA2 | | |
| CD215 | IL-15Rα, Interleukin 15 receptor alpha chain | m IgG2b | 9.55 |
| | (IL-15RA) | | |
| CD217 | IL-17R, CDw217, Interleukin 17 receptor A (IL- | m IgG1 | 23.79 |
| | 17RA) | | |
| CD218a | IL-18 Receptor alpha, IL18Rα, IL-1Rrp1, IL- | m IgG1 | 29.07 |
| | 18R, Interleukin 18 receptor 1 (IL-18R1), IL- | | |
| | 18RA, IL1 receptor-related protein (IL-1Rrp), | | |
| | IL-R5, CDw218a | | |
| CD218b | IL-1RcPL, CDw218b, Interleukin 18 receptor β | m IgG2b | 33.21 |
| | (IL-18Rβ), IL-18 receptor accessory protein (IL- | | |
| | 18RAP, IL-18RAcP), IL-1R accessory protein- | | |
| | like (IL-1RAcPL), IL-1R7 | | |
| CD220 | Insulin R, Insulin receptor (INSR), IR | m IgG2b | 6.10 |
| CD221 | Insulin-like growth factor 1 receptor (IGF1R), | m IgG1 | 4.29 |
| | IGFR, type I IGF receptor (IGF-IR), JTK13 | | |
| CD222 | Cation-independent mannose-6-phosphate | m IgG1 | 12.72 |
| | receptor (M6P-R, CIM6PR, CIMPR, CIMPR), | | |
| | Insulin-like growth factor 2 receptor (IGF2R, | | |
| | IGFIIR, IGF-IIR), MPR1, MPRI | | |
| CD223 | Lymphocyte activation gene 3 (LAG3, LAG-3), | m IgG1 | 14.42 |
| | FDC protein | | |
| CD226 | DNAX accessory molecule 1 (DNAM-1), | m IgG1 | 93.14 |
| | Platelet and T-cell activation antigen 1 (PTA- | | |
| | 1), T lineage-specific activation antigen 1 | | |
| | antigen (TLiSA1) | | |
| CD227 | Mucin 1 (MUC1, MUC-1), DF3 antigen, H23 | m IgG1 | 10.21 |
| | antigen, Peanut-reactive urinary mucin | | |
| | (PUM), Polymorphic epithelial mucin (PEM), | | |
| | Epithelial membrane antigen (EMA), Tumor- | | |
| | associated mucin, Episialin | | |
| CD229 | Lymphocyte antigen 9 (Ly9), T-lymphocyte | m IgG1 | 0.01 |
| | surface antigen Ly-9, Signaling lymphocyte | | |
| | activation molecule family member 3 | | |
| | (SLAMF3), Lgp100, T100 | | |
| CD230 | Prion protein (PrP, PRNP), Major prion | m IgG1 | 95.02 |
| | protein, prP27-30, prP33-35C, PrPc | | |
| CD231 | A15, Tetraspanin 7 (TSPAN7), T-cell acute | m IgG1 | 7.95 |
| | lymphoblastic leukemia-associated antigen 1 | | |
| | (TALLA-1), Transmembrane 4 superfamily | | |
| | member 2 (TM4SF2), Membrane component | | |
| | X chromosome surface marker-1 (MXS1) | | |
| CD234 | Duffy, Duffy antigen/chemokine receptor | m IgG2a | 7.47 |
| | (DARC), Duffy blood group antigen (Dfy, FY), | | |
| | Fy-Glycoprotein, Glycoprotein D | | |
| CD235ab | Glycophorin A/B | m IgG2b | 59.96 |
| CD235a | Glycophorin A (GYPA), Sialoglycoprotein α, | m IgG2b | 0.41 |
| | Sialoglycoprotein A, MN blood group antigen, | | |
| | PAS-2 | | |
| CD238 | B-CAM, Kell blood group glycoprotein (Kel), | m IgG1 | 0.87 |
| | Kell blood group antigen, Endothelin-3- | | |
| | converting enzyme (ECE3), Kell | | |
| CD239 | Rh30CE, Basal cell adhesion molecule (BCAM, | m IgG2a | 1.84 |
| | B-CAM), Lutheran blood group glycoprotein, | | |
| | Lutheran blood group antigen (Lu) | | |
| CD243 | MDR-1, P-gp, GP170, p170, ABC-B1, ABC20, | m IgG2a | 11.58 |
| | CD243, CLCS, PGY1 | | |
| CD244 | 2B4, p38, NKLR2B4, NAIL, Nmrk, SLAMF4 | m IgG1 | 4.36 |
| CD247 | CD3-z, CD3H, CD3Q, CD3Z, T3Z, TCRZ, TCRz, | m IgG1 | 16.16 |
| | Zeta chain | | |
| CD252 | OX40L, OX-40L, TNFSF4, GP34, TXGP1, | m IgG1 | 33.98 |
| | CD134L | | |
| CD253 | TRAIL, TNFSF10, TL2, APO2L, Apo-2L | m IgG1 | 4.38 |
| CD254 | TRANCE, RANKL, TNFSF11, OPGL, ODF, sOdf, | m IgG2b | 6.81 |
| | OPTB2, hRANKL2 | | |
| CD255 | TWEAK, TNFSF12, APO3L | m IgG3 | 11.85 |
| CD257 | BAFF, BLyS, TNFSF13b, TALL1, THANK, | m IgG1 | 60.04 |
| | TNFSF20, ZTNF4 | | |
| CD258 | LIGHT, TNFSF14, LTg, TR2, HVEML | m IgG1 | 12.28 |
| CD261 | DR4, TRAIL-R1, TNFRSF10a, APO2, MGC9365 | m IgG1 | 7.43 |
| CD262 | DR5, TRAIL-R2, KILLER, TNFRSF10b, TRICK2, | m IgG1 | 14.93 |
| | TRICK2A, TRICK2B, TRICKB, ZTNFR9 | | |
| CD263 | DcR1, TRAIL-R3, TRID, TNFRSF10c, LIT | m IgG1 | 6.31 |
| CD264 | TRAIL-R4, DcR2, TNFSF10d, TRUNDD | m IgG1 | 1.34 |
| CD265 | TRANCE-R, RANK, TNFRSF11a, EOF, FEO, | m IgG1 | 5.69 |
| | ODFR, OFE, PDB2 | | |
| CD266 | TWEAK Receptor, TWEAK-R, TNFRSF12A, | m IgG2b | 3.54 |
| | FN14, FGFinducible 14 | | |
| CD267 | TACI, TNFRSF13B, CVID, FLJ39942, | m IgG2a | 4.09 |
| | MGC39952, MGC133214, TNFRSF14B | | |
| CD268 | BAFFR, BR3, TNFRSF13C, TR13C, CD26B, | m IgG2a | 52.42 |
| | BAFF-R, MGC138235 | | |
| CD269 | BCMA, TNFRSF17, BCM | m IgG2a | 3.70 |
| CD270 | HVEM, TR2, HVEA, TNFRSF14, ATAR | m IgG1 | 99.97 |
| CD271 | NGFR (p75), p75NGFR, p75NTR, TNFRSF16, | m IgG1 | 9.06 |
| | Gp80-LNGFR | | |
| CD272 | BTLA, BTLA1, FLJ16065, MGC129743 | m IgG2a | 90.80 |
| CD273 | B7DC, PDL2, PD-L2, PDCD1L2, PDCD1LG2, | m IgG1 | 1.44 |
| | Btdc, CD273, MGC142238, MGC142240, | | |
| | bA574F11.2 | | |
| CD274 | B7H1, B7-H, PDL1, PD-L1, PDCD1LG1, | m IgG1 | 42.25 |
| | PDCD1L1, MGC142294, MGC142296, CD274 | | |
| CD275 | B7H2, B7-H2, ICOSL, B7RP1, B7h, GL50, | m IgG1 | 1.46 |
| | ICOSLG, CD275, LICOS, B7RP-1, ICOS-L, | | |
| | KIAA0653 | | |
| CD276 | B7RP-2, B7H3, B7-H3, 4Ig-B7-H3 | m IgG1 | 16.65 |
| CD277 | BT3.1, BTN3A1, BTF5, MGC141880 | m IgG1 | 99.97 |
| CD278 | ICOS, AILIM, CD278, MGC39850 | m IgG1 | 46.11 |
| CD279 | PD1, SLEB2, PDC1, CD279, hPD-1, PDCD1 | m IgG1 | 16.38 |
| CD281 | TLR1, TIL, rsc786, KIAA0012, MGC104956, | m IgG1 | 4.79 |
| | MGC126311, MGC126312, TIL.LPRS5, | | |
| | DKFZp547I0610, DKFZp564I0682 | | |
| CD282 | TLR2, TIL4, CD282 | m IgG2a | 4.68 |
| CD283 | TLR3, TOLL-like receptor 3 | m IgG2a | 21.75 |
| CD284 | TLR4, TOLL, hToll, ARMD10 | m IgG2a | 5.93 |
| CD286 | TLR6, TOLL-like receptor 6 | m IgG1 | 5.43 |
| CD289 | TLR9, TOLL-like receptor 9 | m IgG1 | 6.84 |
| CD290 | TLR10, TOLL-like receptor 10 | m IgG1 | 1.24 |
| CD292 | BMPR-IA, BMPR1A, ALK3, BIMPR1A, | m IgG1 | 5.76 |
| | 10q23del, ACVRLK3, SKR5 | | |
| CD294 | CRTH2, DP2, PGRD2, G protein-coupled | r IgG2a | 0.53 |
| | receptor 44 (GPR44), DL1R | | |
| CD295 | Leptin R, LEPR, OBR | m IgG2b | 14.68 |
| CD298 | ATP1B3, Na K ATPase β3 subunit, ATPB-3, | m IgG1 | 99.96 |
| | FLJ29027, ATP1β3 | | |
| CD299 | DC-SIGN/L, DC-SIGNR, L-SIGN, DCSIGN- | m IgG2a | 0.02 |
| | related, DCSIGNR, HP10347, DC-SIGN2, | | |
| | MGC47866, MGC12996, CLEC4M | | |
| CD300a | IRC1, IRC2, CLM-8, IRp60, IGSF12, CMRF35H, | m IgG1 | 30.93 |
| | CMRF-35H, CMRF35-H, CMRF-35-H9 | | |
| CD300c | CMRF35A, CMRF-35A, LIR, CLM-6, CMRF35, | m IgG1 | 0.48 |
| | IGSF16, CMRF-35, CMRF35A1, CMRF35-A1 | | |
| CD300e | CMRF35L1, CMRF-35L1, CLM2, CLM-2, IREM2, | m IgG1 | 0.98 |
| | PIgR2, IREM-2, PIgR-2, CD300LE, CMRF35-A5, | | |
| | CMRF35L | | |
| CD300f | IREM-1, IREM1, MAIR-V | m IgG1 | 0.06 |
| CD301 | CLEC10A, MGL1, CLECSF14, HML, MGL | m IgG2a | 9.50 |
| CD302 | CLEC13A, DCL1, BIMLEC | m IgG1 | 0.83 |
| CD303 | BDCA-2, BDCA2, CLEC4C, HECL | m IgG2a | 2.06 |
| CD304 | Neuropilin-1, BDCA-4, NRP1 | m IgG2a | 0.74 |
| CD305 | LAIR1 | m IgG1 | 68.47 |
| CD306 | LAIR2 | m IgG2b | 6.75 |
| CD307a | FcRH1, FCRL1, FCRH, IFGP1, IRTA5 | m IgG1 | 1.53 |
| CD307b | FCRL2, SPAP1, FcRH2, IFGP4, IRTA4 | m IgG1 | 3.73 |
| CD307c | FcRH3, FCRL3, IFGP3, IRTA3, SPAP2 | m IgG1 | 1.16 |
| CD307d | FCRL4, FCRH4, IGFP2, IRTA1 | m IgG2b | 5.59 |
| CD307e | FCRL5, BXMAS1, FCRH5, IRTA2, CD307 | m IgG2a | 0.13 |
| CD309 | VEGFR2, KDR, Flk1 | m IgG1 | 0.01 |
| CD312 | EMR2 | m IgG2b | 15.51 |
| CD314 | NKG2D, KLRK1 | m IgG1 | 1.79 |
| CD317 | BST2, PDCA-1, Tetherin, HM1.24 | m IgG1 | 20.27 |
| CD318 | CDCP1, SIMA135 | m IgG2b | 3.24 |
| CD319 | CRACC, CS1, SLAMF7 | m IgG2b | 8.75 |
| CD321 | JAM1, JAM, JAM-A, F11R | m IgG1 | 71.57 |
| CD324 | E-Cadherin, CDH1 | m IgG1 | 2.73 |
| CD325 | N-Cadherin, CDH2 CDw325, NCAD, CDH2 | m IgG1 | 2.55 |
| CD326 | Ep-CAM, MK-1, KSA, EGP40, TROP1, TACSTD1 | m IgG1 | 1.11 |
| CD328 | Siglec-7, p75/AIRM, Siglec7, AIRM-1 | m IgG1 | 2.06 |
| CD329 | Siglec-9 | m IgG2a | 2.05 |
| CD332 | FGFR2, BEK, K-SAM, KGFR | m IgG1 | 0.01 |
| CD333 | FGFR3, ACH, CEK2 | m IgG1 | 5.73 |
| CD334 | FGFR4, TKF, JTK2 | m IgG1 | 1.17 |
| CD335 | NKp46, NCR1, Ly94 | m IgG1 | 0.00 |
| CD336 | NKp44, NCR2, Ly-95 homolog, Ly95 | m IgG2b | 0.06 |
| CD337 | NKp30, NCR3, Ly-117 | m IgG1 | 0.43 |
| CD338 | ABCG2, ABCP, MXR, BCRP, Brcp1 | m IgG2b | 1.18 |
| CD339 | Jagged-1, JAG1, JAGL1, hJ1 | m IgG2b | 1.36 |
| CD340 | HER2/neu, Her-2, Neu, p185HER2, ERB-B2, | m IgG1 | 0.85 |
| | erbB2/HER-2 | | |
| CD344 | Frizzled-4, FZD4, EVR1, FEVR, Frizzled | m IgG1 | 10.14 |
| | homolog 4, Fz-4, hFz-4, FzE4 | | |
| CD352 | NTB-A, SLAMF6, Ly108 | m IgG1 | 99.90 |
| CD353 | SLAMF8, BLAME | m IgG1 | 4.46 |
| CD354 | TREM-1, TREM1 | m IgG1 | 2.46 |
| CD355 | CRTAM, Cytotoxic and regulatory T-cell | m IgG2a | 1.80 |
| | molecule | | |
| CD357 | TNFRSF18, Tumor necrosis factor receptor | m IgG1 | 3.63 |
| | superfamily, member 18, GITR, AITR | | |
| CD360 | IL-21R, IL21R | m IgG1 | 21.68 |
| CD362 | Syndecan-2 | r IgG2b | 0.03 |
| CD363 | S1PR1, Sphingosine-1-phosphate receptor 1, | m IgG2b | 2.67 |
| | EDG-1 | | |

Total 348
Positive 302

TABLE 3
Flow cytometric analysis of the multilineage progenitor cells derived from
CD8⁺ PBMCs which have been cultured according to the method
of the present invention Proteins expression of CD8⁺ PBMCs
by Flow Cytometry Analysis

| CD markers | Isotype | % of positive cells |
|---|---|---|
| αβTCR | m IgG1 | 98.91 |
| CLA | r IgM | 2.37 |
| EGFR | m IgG1 | 0.50 |
| HER-2 (c-new) | m IgG1 | 2.81 |
| HLA-A,B,C | m IgG2a | 99.94 |
| HLA-A2 | m IgG2b | 2.92 |
| HLA-DQ | m IgG1 | 1.72 |
| HLA-DR | m IgG2a | 12.41 |
| HLA-DR,DP,DQ | m IgG2a | 4.59 |
| Integrin-β7 | r IgG2a | 13.07 |
| MIC A/B | m IgG2a | 0.15 |
| MHC Class I free chain without | m IgG1 | 0.01 |
| beta2 microglobulin | | |
| SSEA-1 | m IgM | 0.63 |
| SSEA-3 | r IgM | 1.79 |
| SSEA-4 | m IgG3 | 0.54 |
| TRA-1-60 | m IgM | 3.63 |
| TRA-1-81 | m IgM | 40.23 |
| Vβ8 | m IgG2b | 4.40 |
| Vβ23 | m IgG2a | 0.23 |

Total 19
Positive 13

TABLE 4
Flow cytometric analysis of the multilineage progenitor cells derived from CD8⁺ PBMCs
which have been cultured according to the method of the present invention
CD markers expression of CD8⁺ PBMCs by Flow Cytometry Analysis

| CD markers | Alternate names | Isotype | % of positive cells |
|---|---|---|---|
| CD1a | R4, T6, Leu-6, HTA1 | m IgG2a | 14.51 |
| CD1b | R1, T6 | m IgG1 | 0.01 |
| CD1c | BDCA-1, R7, T6, M241 | m IgG1 | 0.04 |
| CD1d | R3, R3G1 | m IgG2b | 3.61 |
| CD2 | T11, LFA-2, SRBC-R, E-rosette R, Erythrocyte R | m IgG2a | 99.85 |
| CD3 | T3 | m IgG1 | 99.71 |
| CD4 | T4, Leu-3, L3T4, Leu-3a, W3/25 | m IgG1 | 0.91 |
| CD5 | T1, Tp67, Leu-1, Ly-1 | m IgG2a | 99.44 |
| CD6 | T12, TP120 | m IgG1 | 99.94 |
| CD7 | gp40, Leu-9, TP41 | m IgG2a | 98.95 |
| CD8 | T8, Leu-2 | m IgG2a | 83.42 |
| CD8a | type I glycoprotein | m IgG1 | 99.83 |
| CD8b | Lyt3 | m IgG2a | 91.27 |
| CD9 | p24, MRP-1, DRAP-27, DRAP-1 | m IgG1 | 47.86 |
| CD10 | CALLA, NEP, gp100, EC 3.4.24.11, MME | m IgG2b | 1.66 |
| CD11a | LFA-1, integrin αL, ITGAL, LFA-1α | m IgG1 | 96.16 |
| CD11b | Mac-1, integrin αM, CR3, ITGAM, Mo1, C3niR | m IgG2a | 9.39 |
| CD11c | p150, 95, CR4, integrin αX, ITGAX, AXb2 | m IgG2a | 49.05 |
| CD13 | APN, gp150, Amniopeptidase N, ANPEP, AAP, | m IgG2a | 1.85 |
| | APM, LAP1, P150, PEPN, EC 3.4.11.2 | | |
| CD14 | LPS-Receptor | m IgG2a | 5.04 |
| CD15 | Lewis X, Lex, SSEA-1, 3-FAL, X-Hapten, FUT4 | m IgM | 47.49 |
| CD15s | Sialyl Lewis X | m IgM | 0.00 |
| CD16 | FCRIIIA, CD16a | m IgG2a | 52.38 |
| CD16b | FCRIIIB, FcγRIIIB | m IgG2a | 19.39 |
| CD17 | Lactosylceramide, LacCer | m IgG2a | 7.92 |
| CD18 | Integrin β2, ITGB2, CD11a, b, c β-subunit | m IgG1 | 99.91 |
| CD19 | B4 | m IgG1 | 0.19 |
| CD20 | B1, Bp35, Ly-44 | m IgG2b | 4.31 |
| CD21 | CR2, EBV-R, C3dR | m IgG2a | 8.94 |
| CD22 | BL-CAM, Siglec-2 | m IgG1 | 0.05 |
| CD23 | FcεRII, BLAST-2, FceRII, B6, Leu-20 | m IgG1 | 0.09 |
| CD24 | BA-1, HAS, HSA, BBA-1 | m IgG2a | 1.42 |
| CD25 | p55, IL-2Rα, Tac antigen, Tac, TCGFR | m IgG2a | 32.61 |
| CD26 | DPP IV ectoenzyme, DPP IV, ADA binding | m IgG1 | 89.38 |
| | protein, ADCP2, TP103 | | |
| CD27 | T14, S152, TNFRSF7, TP55 | m IgG2a | 94.53 |
| CD28 | Tp44, T44 | m IgG1 | 96.11 |
| CD29 | Integrin β1, platelet GPIIa, ITGB1, GP | m IgG1 | 99.98 |
| CD30 | Ki-1, Ber-H2, TNFRSF8 | m IgG2a | 7.82 |
| CD31 | PECAM-1, endocam, GPIIa, Platelet | m IgG1 | 84.74 |
| | endothelial cell adhesion molecule, PECA1 | | |
| CD32 | FcγRII | m IgG2a | 5.47 |
| CD33 | p67, Siglec-3, My9, gp67, Sialic acid-binding | m IgG2a | 29.02 |
| | Ig-like lectin 3, Myeloid cell surface antigen | | |
| | CD33 | | |
| CD34 | gp105-120, Mucosialin, My10, Hematopoietic | m IgG2a | 62.77 |
| | progenitor cell antigen 1 (HPCA1) | | |
| CD35 | CR1, C3b/C4b receptor, Immune adherence | m IgG2a | 6.31 |
| | receptor, Complement receptor 1 | | |
| CD36 | GPIV, OKM5 antigen, PASIV, Glycoprotein IIIb | m IgM | 25.23 |
| | (GpIIIb), Glycoprotein IV (GPIV), Fatty acid | | |
| | translocase (FAT), SCARB3, GP88, Platelet | | |
| | glycoprotein 4 | | |
| CD37 | gp 52-40, Tspan-26, Leukocyte antigen CD37, | m IgG1 | 0.01 |
| | Tetraspanin-26, TSPAN26 | | |
| CD38 | T10, ADP-ribosyl cyclase, Cyclic ADP-ribose | m IgG2a | 77.81 |
| | hydrolase 1 | | |
| CD39 | NTPDase-1, gp80, EC3.6.1.5, Ectonucleoside | m IgG2a | 87.73 |
| | triphosphate diphosphohydrolase 1 | | |
| | (ENTPD1), ATPdehydrogenase | | |
| CD40 | Bp50, TNFRSF5, MGC9013, Tumor necrosis | m IgG2a | 93.11 |
| | factor receptor superfamily member 5 | | |
| CD41 | ITGA2B, GPIIb, Integrin αIIb, Platelet | m IgG2a | 76.30 |
| | membrane glycoprotein IIb, Integrin α2b, | | |
| | Human Platelet Antigen-3 | | |
| | (HPA-3) | | |
| CD41a | Integrin alpha Iib, platelet GPIIb | m IgG2a | 48.36 |
| CD41b | fibrinogen receptor, gpIIb/IIIa, integrin alpha | mIgG3 | 29.29 |
| | IIb, ITGA2b | | |
| CD42a | GPIX, GP9, Platelet glycoprotein IX | m IgG2a | 22.25 |
| CD42b | gpIbα, GPIba, Platelet glycoprotein Ib α | m IgG2a | 23.71 |
| CD42d | Glycoprotein V, GPV, Platelet glycoprotein V | m IgG2a | 23.70 |
| CD43 | gpL115, Sialophorin, Leukosialin, | mIgG1 | 99.98 |
| | Galactoglycoprotein, SPN | | |
| CD44 | H-CAM, Pgp-1, EMCR III, CD44s, Hermes | m IgG2b | 99.93 |
| | antigen, ECMRII, Phagocytic glycoprotein 1, | | |
| | Extracellular matrix receptor III, GP90 | | |
| | Lymphocyte homing/adhesion receptor, | | |
| | Hyaluronate receptor | | |
| CD45 | Leukocyte Common Antigen (LCA), T200, | m IgG1 | 99.98 |
| | B220, Ly5, Protein tyrosine phosphatase | | |
| | receptor type C (PTPRC) | | |
| CD45RA | PTPRC | m IgG2b | 81.83 |
| CD45RB | PTPRC | m IgG2b | 99.92 |
| CD45RO | UCHL-1 | m IgG2a | 75.26 |
| CD46 | Membrane Cofactor Protein (MCP), | m IgG1 | 99.95 |
| | Trophoblast leukocyte common antigen, | | |
| | TRA2.10 | | |
| CD47 | IAP, neurophilin, gp42, OA3, MER6 | m IgG1 | 99.98 |
| CD48 | Blast-1, BCM1, Sgp-60, SLAMF2, Hulym3, OX- | m IgG1 | 99.48 |
| | 45, MEM-102 | | |
| CD49a | VLA-1α, Integrin α1, VLA-1, ITGA1 | m IgG1 | 39.64 |
| CD49b | VLA-2α, gpIa, Integrin α2, VLA-2, ITGA2 | m IgG1 | 77.96 |
| CD49c | VLA-3α, Integrin α3, VLA-3, ITGA3, GAPB3, | m IgG1 | 0.00 |
| | Galactoprotein B3, MSK18, Very Common | | |
| | Antigen-2 (VCA-2) | | |
| CD49d | VLA-4α, Integrin α4, VLA-4, ITGA4 | m IgG1 | 99.27 |
| CD49e | VLA-5α, Integrin α5, VLA-5, ITGA5, | m IgG3 | 69.28 |
| | Fibronectin receptor | | |
| CD49f | VLA-6α, Integrin α6, VLA-6, ITGA6, gpI | r IgG2a | 19.78 |
| CD50 | ICAM-3 | m IgG2a | 99.96 |
| CD51/61 | vitronectin R, Integrin αv, VNR-α, Vitronectin- | m IgG2a | 2.87 |
| | Rα, ITGAV, Integrin αvβ3 | | |
| CD52 | CAMPATH-1, HE5, Epididymal secretory | m IgG2b | 99.73 |
| | protein E52, HES | | |
| CD53 | OX-44, MCR, TSPAN25, MOX44, Tetraspanin- | m IgG2a | 99.93 |
| | 25 | | |
| CD54 | ICAM-1 | m IgG2a | 50.75 |
| CD55 | Decay Accelerating Factor for Complement | m IgG2a | 99.28 |
| | (DAF) | | |
| CD56 | Leu-19, NKH-1, Neural Cell Adhesion | m IgG2a | 46.22 |
| | Molecule (NCAM) | | |
| CD57 | HNK-1, Leu-7, β-1,3-glucuronyltransferase 1, | m IgM | 42.76 |
| | Glucuronosyltransferase P, | | |
| | galactosylgalactosylxylosylprotein 3-β- | | |
| | glucuronosyltransferase 1 | | |
| CD58 | LFA-3 | m IgG2a | 0.02 |
| CD59 | Protectin, H19, 1F-5Ag, MIRL, MACIF, P-18 | m IgG2a | 99.98 |
| CD60b | 9-O-sialyl GD3 | m IgM | 0.45 |
| CD61 | GP IIIa, Integrin β3 | m IgG2a | 4.58 |
| CD62E | E-selectin, ELAM-1, LECAM-2 | m IgG2a | 0.72 |
| CD62L | L-selectin, LECAM-1, LAM-1, Leu-8, TQ1, MEL- | m IgG2a | 74.15 |
| | 14 | | |
| CD62P | P-selectin, GMP-140, PADGEM | m IgG2a | 5.75 |
| CD63 | LIMP, MLA1, LAMP-3, ME491, gp55, NGA, | m IgG2a | 96.52 |
| | OMA81H, TSPAN30, Granulophysin, | | |
| | Melanoma 1 antigen | | |
| CD64 | FcγRI, FcR I | m IgG2a | 1.51 |
| CD65 | Ceramide-dodecasaccharide, VIM2, | m IgM | 13.91 |
| | Fucoganglioside (Type II) | | |
| CD65s | Sialylated poly-N-acetyllactosamine, | m IgM | 26.46 |
| | Sialylated-CD65, VIM2 | | |
| CD66 | | m IgG2a | 1.93 |
| CD66abce | | m IgG2a | 0.23 |
| CD66a | NCA-160, BGP (Biliary glcoprotein), BGP1, | m IgG2a | 0.01 |
| | BGPI, CEACAM1 | | |
| CD66b | CD67, CGM6, NCA-95, CEACAM8 | m IgM | 0.01 |
| CD66c | NCA, NCA-50/90, CEAL, CEACAM6 | m IgG2a | 14.40 |
| CD68 | gp110, Macrosialin, SCARD1 | m IgG2b | 1.80 |
| CD69 | AIM, VEA, MLR3, EA 1, gp34/28, CLEC2C, BL- | m IgG2a | 33.44 |
| | AP26 | | |
| CD70 | Ki-24, CD27L, TNFSF7, CD27LG | m IgG1 | 6.79 |
| CD71 | TfR, T9, TFRC, Transferrin receptor, TRFR | m IgG2a | 3.86 |
| CD72 | Lyb-2, Ly-32.2, Ly-19.2 | m IgG2b | 5.49 |
| CD73 | NT5E, Ecto-5′-nuclotidase, E5NT, NT5, NTE, | m IgG2a | 71.99 |
| | eN, eNT | | |
| CD74 | li, invariant chain, DHLAG, HLADG, Ia-γ | m IgG2a | 35.37 |
| CD75 | lactosamines, ST6GAL1, MGC48859, SIAT1, | m IgM | 0.02 |
| | ST6GALL, ST6N, ST6 β-Galactosamide α-2,6- | | |
| | sialyltranferase, | | |
| | Sialo-masked lactosamine, Carbohydrate of | | |
| | α2,6 sialyltransferase | | |
| CD77 | Pk Ag, BLA, CTH, Gb3, Pk blood groupBLA, | m IgM | 52.99 |
| | A14GALT (Δ1,4-Galactosyltransferase), | | |
| | A4GALT1, Gb3S, P(k), P1, PK A4GALT, Pk | | |
| | antigen, CTH/Gb3A4GALT1, Gb3S, PK, | | |
| | P1 | | |
| CD79a | Igα, MB1, IGA (Immunoglobulin-associated a), | m IgG2a | 4.35 |
| | MB-1 | | |
| CD79b | B29, Igβ (Immunoglobulin-associated β) | m IgG2a | 6.17 |
| CD80 | B7, B7-1, BB1, CD28LG, CD28LG1, L AB7 | m IgG2a | 1.40 |
| CD81 | TAPA-1, S5.7 | m IgG2a | 1.29 |
| CD83 | HB15, BL11 | m IgG2a | 1.14 |
| CD84 | GR6, SLAMF5, LY9B, p75, hly9-β | m IgG2a | 0.00 |
| CD85a | ILT5, LIR3, HL9, LILRB3 (Leukocyte | r IgG2b | 15.59 |
| | immunoglobulin-like receptor, subfamily B | | |
| | (with TM and ITIM domains), member 3, LIR- | | |
| | 3, MGC138403, PIRB, XXbac-BCX105G6.7 | | |
| CD85d | ILT4, LILRB2 (Leukocyte immunoglobulin-like | r IgG2b | 5.48 |
| | receptor, subfamily B (with TM and ITIM | | |
| | domains), member 2, LIR2, MIR10, MIR-10 | | |
| CD85f | LIT11, LILRA5, XXbac-BCX403H19.2, LIR9, | m IgG2a | 6.66 |
| | LILRB7 (Leukocyte immunoglobulin-like | | |
| | receptor, subfamily B (with TM and ITIM | | |
| | domains), member 7 | | |
| CD85g | ILT7, LILRA4 (Leukocyte immunoglobulin-like | m IgG2a | 0.31 |
| | receptor, subfamily A (with TM domain), | | |
| | member 4, MGC129597, MGC129598, LIR4 | | |
| CD85h | ILT1, LILRA2 (Leukocyte immunoglobulin-like | r IgG2b | 1.75 |
| | receptor, subfamily A (with TM domain), | | |
| | member 2, LIR7, LIR-7, XXbac-BCX85G21.2, | | |
| | ILT-1 | | |
| CD85i | LILRA1 (Leukocyte immunoglobulin-like | m IgG2b | 0.03 |
| | receptor), subfamily A (with TM domain), | | |
| | member 1, LIR6, LIR-6, MGC126563 | | |
| CD85j | ILT2, LILRB1 (Leukocyte immunoglobulin-like | m IgG1 | 15.73 |
| | receptor, subfamily B (with TM and ITIM | | |
| | domains), member 1, FLJ37515, LIR-1, LIR1, | | |
| | MIR-7, MIR7 | | |
| CD85k | ILT3, LILRB4 (Leukocyte immunoglobulin-like | m IgG2a | 0.91 |
| | receptor, subfamily B (with TM and ITIM | | |
| | domains), member 4, LIR-5, HM18, LIR5, | | |
| | LILRB5 | | |
| CD86 | B70, B7-2, CD28LG2, LAB72, MGC34413 | m IgG2b | 3.47 |
| CD87 | UPA-R, PLAUR, URKR | m IgG2a | 0.13 |
| CD88 | C5aR, C5aR C5R1, C5R1, C5AR, C5A | m IgG2a | 7.00 |
| CD89 | FcaR, IgA R | m IgG2a | 4.47 |
| CD90 | Thy-1 | m IgG2a | 1.57 |
| CD91 | α2M-R, LRP, LRP1, α2MR, APOER, APR | m IgG2a | 2.39 |
| CD92 | SLC44A1, CTL1, CHTL1, RP11-287A8.1, p70, | m IgG2b | 11.42 |
| | CDw92 | | |
| CD93 | C1qRp, C1QR1, C1qRP, MXRA4, C1qR(P), | m IgM | 2.17 |
| | Dj737e23.1, GR11 | | |
| CD94 | Kp43, KLRD1 | m IgG2a | 6.94 |
| CD95 | Fas, APO-1, TNFRSF6, CD178, FASLG, CD95L, | m IgG1 | 69.33 |
| | APT1LG1, APT1, FAS1, FASTM, ALPS1A, | | |
| | TNFSF6, FASL | | |
| CD96 | TACTILE, MGC22596 | m IgG1 | 72.19 |
| CD97 | EMR1, BL-KDD/F12, TM&LN1 | m IgG2a | 0.03 |
| CD98 | 4F2, FRP-1, RL-388, SLC3A2, 4F2HC, 4T2HC, | m IgG1 | 76.80 |
| | MDU1, NACAE | | |
| CD99 | MIC2, E2, MIC2, MIC2X, MIC2Y, HBA71, | m IgG2a | 99.82 |
| | MSK5X | | |
| CD99R | E2, CD99 Mab restricted | m IgM | 90.77 |
| CD100 | SEMA4D, SEMAJ, coll-4, C9orf164, FLJ33485, | m IgM | 97.66 |
| | FLJ34282, FLJ39737, FLJ46484, M-sema-G, | | |
| | MGC169138, MGC169141, SEMAJ | | |
| CD101 | BB27, V7, P126, IGSF2, BA27, BPC#4, V7-LSB | m IgG2a | 0.02 |
| CD102 | ICAM-2, Ly60 | m IgG2a | 99.66 |
| CD103 | HML-1, Integrin αE, alEL, ITGAE, OX62, HML1 | m IgG2a | 8.05 |
| CD104 | TSP-180, Integrin β4, TSP1180, ITGB4 | r IgG2b | 0.10 |
| CD105 | Endoglin, ENG, HHT1, ORW, SH-2 | m IgG2a | 88.58 |
| CD106 | VCAM-1, INCAM-110, V-CAM, INCAM-100 | m IgG1 | 2.46 |
| CD107a | LAMP-1, LAMPA, CD107a, LGP120 | m IgG2a | 87.20 |
| CD107b | LAMP-2, LAMPB | m IgG2a | 6.94 |
| CD108 | SEMA7A, JMH blood group antigen, JMH | m IgM | 72.15 |
| CD109 | 8A3, 7D1, E123, Platelet activation factor, | m IgG2a | 13.29 |
| | 8As, 150 kD TGF-β-1-binding protein, Platelet- | | |
| | specific Gov antigen | | |
| CD110 | MPL, TPO-R, C-MPL | m IgG2a | 9.84 |
| CD111 | PRR1, Nectin-1, PVRL1, HveC, HIgR, CLPED1, | m IgG2a | 40.87 |
| | Hve C1 | | |
| CD112 | PRR2, Nectin-2, HveB, PVRL2 | m IgG2a | 27.96 |
| CD114 | G-CSFR, CSF3R, HG-CSFR | m IgG2a | 0.07 |
| CD115 | CSF-1R, M-CSFR, c-fms, FMS, FIM2 | r IgG1 | 6.66 |
| CD116 | GM-CSFRα, GM-CSFRa, CDw116, CSF2R, | m IgG2a | 8.57 |
| | CSF2RAX, CSF2RAY, CSF2RX, CSF2RY, GM-CSF- | | |
| | R-α, GMCSFR, GMR, MGC3848, MGC4838 | | |
| CD117 | c-kit, SCFR, PBT | m IgG1 | 32.63 |
| CD118 | LIFR, gp190, SJS2, STWS, SWS | m IgG2a | 4.12 |
| CD119 | IFNγR, IFNγRa, CDw119, IFNGR1, IFNγRa | m IgG2a | 75.80 |
| CD120a | TNFR-I, p55, TNFRSF1A, CD120a, FPF, | m IgG2a | 6.77 |
| | MGC19588, TBP1, TNF-R, TNF-R55, TNFAR, | | |
| | TNFR1, TNFR55, TNFR60, p55-R, p60 | | |
| CD120b | TNFR-II, p80, TNFRSF1B, p75, TNFR p80 | r IgG2b | 63.25 |
| CD121a | IL-1R type I, IL-1RI, IL1R, CD121A, D2S1473, IL- | m IgG2a | 15.40 |
| | 1R-α, IL1RA, P80 | | |
| CD121b | IL-1R type II, IL-1RII | m IgG2a | 8.51 |
| CD122 | IL-2Rβ, IL2RB, p70-75 | m IgG2a | 0.49 |
| CD123 | IL-3Rα, IL3RA, CD123, IL3R, IL3RAY, IL3RX, | m IgG2a | 3.11 |
| | IL3RY, MGC34174, hIL-3Ra | | |
| CD124 | IL-4Rα, IL4R | m IgG2a | 3.63 |
| CD125 | IL-5Rα, CDw125, IL5RA | m IgG2a | 35.03 |
| CD126 | IL-6Rα, IL6R | m IgG2a | 0.33 |
| CD127 | IL-7R, IL-7Rα, IL7R, p90 | m IgG2a | 98.86 |
| CD129 | IL-9R, IL-9Rα | m IgG2b | 9.58 |
| CD130 | gp130, IL-6Rβ, IL6ST, IL6ST, IL6-β | m IgG2a | 56.58 |
| CD131 | CSF2RB, IL3RB, IL5RB, CDw131, IL-3Rβ, | m IgG2a | 3.20 |
| | common β chain, IL-3R common β | | |
| CD132 | Common γ chain, IL-2Rγ, IL2RG | r IgG2a | 77.05 |
| CD133 | AC133, PROML1, Prominin 1, Hematopoietic | m IgG2a | 2.61 |
| | stem cell antigen, prominin-like 1 | | |
| CD134 | OX-40, TNFRSF4 | m IgG2a | 5.49 |
| CD135 | Flt3/Flk2, STK-1 | m IgG2a | 2.55 |
| CD136 | MSP-R, RON, p158-ron, CDw136, MST1R | m IgG2a | 99.39 |
| CD137 | 4-1BB, TNFRSF9, ILA | m IgG2a | 5.26 |
| CD137 | 4-1BB Ligand | m IgG2a | 12.49 |
| Ligand | | | |
| CD138 | Syndecan-1, Heparan sulfate proteoglycan | m IgG2a | 7.81 |
| CD140a | PDGFRA, PDGF α Receptor, PDGFRα | m IgG2a | 28.32 |
| CD140b | PDGFRB, PDGF β Receptor, PDGFRβ | m IgG2a | 10.70 |
| CD141 | Thrombomodulin, THBD, Fetomodulin | m IgG2a | 9.46 |
| CD142 | Tissue Factor (TF), Factor III, Thromboplastin | m IgG2a | 1.93 |
| CD144 | VE-Cadherin, Cadherin-5 | m IgG2a | 4.84 |
| CD146 | MUC18, S-endo, MCAM, Mel-CAM, Endo- | m IgG2a | 14.92 |
| | CAM | | |
| CD147 | Neurothelin, basigin, EMMPRIN, BSG, M6, | m IgG2a | 0.00 |
| | OX47, TCSF | | |
| CD148 | HPTP-eta, p260, DEP-1, HPTP-η, SCC1, PTPRJ | m IgG2a | 6.40 |
| CD150 | SLAM, IPO-3 | m IgG2a | 55.38 |
| CD151 | PETA-3, Tspan-24, RAPH, SFA-1 | m IgG2a | 0.04 |
| CD152 | CTLA-4 | m IgG2a | 0.60 |
| CD153 | CD30L, TNFSF8, TNSF8 | m IgG2b | 43.50 |
| CD154 | CD40L, T-BAM, gp39, TRAP, TNFSF5, TRAP-1, | m IgG2a | 6.31 |
| | IMD3 | | |
| CD155 | PVR, Necl-5, PVS, TAGE4, HVED, NECL5 | m IgG2a | 2.36 |
| CD156b | TACE, ADAM17, cSVP | m IgG2a | 65.20 |
| CD156c | ADAM10, MADM, kuz | m IgG2b | 99.77 |
| CD157 | BST-1, Bp3, Mo5 | m IgG1 | 4.07 |
| CD158a | KIR2DL1, p58.1, NKAT1 | m IgG2b | 7.77 |
| CD158b | p58.2, KIR2DL2/L3, NKAT2 | m IgG2a | 3.80 |
| CD158d | KIR2DL4, KIR103AS, KIR103 | m IgG2a | 22.15 |
| CD158e1 | KIR3DL1, NKB1, NKB1B, p70 | m IgG2a | 2.44 |
| CD158f | KIR2DL5A, KIR2DL5 | m IgG2a | 1.86 |
| CD159a | NKG2A, KLRC1 | m IgG2a | 4.27 |
| CD159c | NKG2C, KLRC2 | m IgG2a | 4.97 |
| CD160 | BY55, NK1, NK28 | m IgM | 41.05 |
| CD161 | NKR-P1A, KLRB1, NKR | m IgG2a | 36.73 |
| CD162 | PSGL-1 | m IgG2a | 99.51 |
| CD163 | M130, GHI/61, D11, RM3/1 | m IgG2a | 1.12 |
| CD164 | MGC-24, MUC-24, Endolyn | m IgG2a | 98.37 |
| CD165 | AD2, gp37 | m IgG1 | 93.62 |
| CD166 | ALCAM, KG-CAM, SC-1, BEN, DM-GRASP | m IgG2a | 19.73 |
| CD167a | DDR1, trkE, cak | m IgG3 | 69.93 |
| CD169 | Sialoadhesin, Siglec-1 | m IgG2a | 61.59 |
| CD170 | Siglec-5, CD33-like2 | m IgG1 | 4.77 |
| CD171 | L1CAM, N-CAM L1, L1 antigen, HSAS, HSAS1, | m IgG2a | 3.34 |
| | MASA, MIC5, S10, SPG1, NILE | | |
| CD172a | SIRP alpha, BIT, MFR, MYD-1, P84, SHPS-1, | m IgG2a | 0.08 |
| | SHPS1, SIRPα2, SIRPa | | |
| CD172b | SIRPβ, SIRPβ1 | m IgG2a | 2.55 |
| CD172g | SIRPγ, SIRPβ2, SIRPγ, SIRP-B2, bA77C3.1 | m IgG1 | 98.00 |
| CD177 | NB1, HNA-2a, NB1gp, Neutrophil-specific | m IgG2a | 1.37 |
| | antigen 1, PRV1 | | |
| CD178 | CD95L, TNFSF6, Fas Ligand, FasL, APT1LG1 | m IgG2a | 1.67 |
| CD179a | VpreB, IGVPB, VPREB1 | m IgG2a | 4.56 |
| CD179b | Igλ5, λ5, 14.1, IGL5, IGGL1, IGO, lambda5 | m IgG2a | 33.94 |
| CD180 | RP105, LY64, Bgp95, Ly78 | m IgG2a | 3.52 |
| CD181 | CDw128A, IL-8RA, (formerly CD128a) CXCR1, | m IgG2b | 5.99 |
| | IL-8Rα | | |
| CD182 | CDw128B, IL-8RB, (formerly CD128b) CXCR2, | m IgG2a | 5.46 |
| | IL-8Rβ, CMKAR2, IL8R2 | | |
| CD183 | CXCR3, GPR9, CKR-L2, CMKAR3, IP10, Mig-R, | m IgG2a | 98.67 |
| | TAC | | |
| CD184 | CXCR4, Fusin, LESTR, NPY3R, CMKAR4, HM89, | m IgG2a | 99.96 |
| | FB22, LCR1 | | |
| CD185 | CXCR5, BLR1, MDR15, MGC117347 | m IgG2b | 11.35 |
| CD186 | CXCR6, CDw186, STRL33, TYMSTR, BONZO | m IgG2b | 16.65 |
| CD191 | CCR1, CKR1, CKR-1, HM145, CMKBR1, | m IgG2b | 0.02 |
| | SCYAR1, MIP-1αR, RANTES-R | | |
| CD192 | CCR2, CKR2, CCR2A, CCR2B, CKR2A, CKR2B, | m IgG2a | 49.71 |
| | CMKBR2, MCP-1-R, CC-CKR-2, FLJ78302, | | |
| | MGC103828, MGC111760, MGC168006 | | |
| CD193 | CCR3, CKR3, CMKBR3, CC-CKR-3, MGC102841 | m IgG2b | 22.49 |
| CD194 | CCR4, CC-CKR-4, CKR4, CMKBR4, ChemR13, | m IgG2b | 16.84 |
| | HGCN | | |
| CD195 | CCR5, CMKBR5, IDDM22, CC-CKR-5, FLJ78003 | m IgG2a | 49.95 |
| CD196 | CCR6, LARC receptor, DRY6, BN-1, DCR2, | m IgG2a | 22.14 |
| | CKRL3, GPR29, CKR-L3, CMKBR6, GPRCY4, | | |
| | STRL22, CC-CKR-6 | | |
| CD197 | EBI-1, BLR-2, CMKBR7, CCR7 (formerly | r IgG2a | 61.47 |
| | CDw197) | | |
| CD198 | CCR8, CKR-L1, CKRL1, CMKBR8, CMKBRL2, | r IgG2b | 6.36 |
| | CY6, GPR-CY6, TER1 | | |
| CD199 | CCR9, GPR28, GPR-9-6 | m IgG2a | 15.48 |
| CD200 | OX2, MRC, MOX1, MOX2 | m IgG2a | 18.96 |
| CD201 | EPC-R, PROCR, CCCA, CCD41, MGC23024, | r IgG1 | 0.00 |
| | bA42O4.2 | | |
| CD202b | Tie2 (Tek), TEK, VMCM, TIE-2, VMCM1 | m IgG2a | 2.34 |
| CD203c | E-NPP3, PD-1b, PDNP3, B10, PDIβ | m IgG2a | 0.64 |
| CD204 | MSR, MSR1, SR-A, phSR1, phSR2, SCARA1 | m IgG2b | 2.70 |
| CD205 | DEC-205, CLEC13B, GP200-MR6, LY75 | m IgG2b | 11.99 |
| CD206 | Mannose receptor C type-1 (MRC1), | m IgG2a | 4.08 |
| | Macrophage mannose receptor (MMR), C- | | |
| | type Lectin domain family 13 member D | | |
| | (CLEC13D) | | |
| CD207 | Langerin, C-type Lectin domain family 4 | m IgG2a | 2.73 |
| | member K (CLEC4K) | | |
| CD208 | DC-LAMP, Lysosomal-associated membrane | m IgG2a | 0.00 |
| | protein 3 (LAMP3), DCLAMP, LAMP, TSC403 | | |
| CD209 | Dendritic cell-specific ICAM-3-grabbing non- | r IgG2a | 3.87 |
| | integrin (DC-SIGN), DC-SIGN1, CDSIGN, C-type | | |
| | lectin domain family 4 member L (CLEC4L), | | |
| | HIV gp120-binding protein | | |
| CD210 | IL-10R | r IgG2a | 53.03 |
| CD210a | Interleukin 10 Receptor A (IL-10RA, IL-10R1), | m IgG2a | 6.18 |
| | IL-10Rα | | |
| CD210b | CRF2-4, Interleukin 10 Receptor B (IL-10RB, IL- | m IgG2a | 5.35 |
| | 10R2), IL-10Rβ, D21S58, CRFB4 | | |
| CD212 | IL-12Rβ1, IL12RB1, IL-12Rb1, Interleukin 12 | m IgG2a | 56.60 |
| | receptor β1 chain (IL-12β1), IL-12β, CD212b1 | | |
| CD213a1 | Interleukin 13 receptor α1 chain (IL-13Rα1), | m IgG2b | 3.13 |
| | NR4 | | |
| CD213a2 | IL12Rα2, IL-13Ra2, Interleukin 13 receptor α2 | m IgG1 | 7.32 |
| | chain (IL-13Rα2), interleukin-13-binding | | |
| | protein (IL13BP), IL13RA2 | | |
| CD215 | IL-15Rα, Interleukin 15 receptor alpha chain | m IgG2b | 10.91 |
| | (IL-15RA) | | |
| CD217 | IL-17R, CDw217, Interleukin 17 receptor A (IL- | m IgG1 | 42.08 |
| | 17RA) | | |
| CD218a | IL-18 Receptor alpha, IL18Rα, IL-1Rrp1, IL- | m IgG2a | 72.49 |
| | 18R, Interleukin 18 receptor 1 (IL-18R1), IL- | | |
| | 18RA, IL1 receptor-related protein (IL-1Rrp), | | |
| | IL-R5, CDw218a | | |
| CD218b | IL-1RcPL, CDw218b, Interleukin 18 receptor β | m IgG2b | 74.91 |
| | (IL-18Rβ), IL-18 receptor accessory protein (IL- | | |
| | 18RAP, IL-18RAcP), IL-1R accessory protein- | | |
| | like (IL-1RAcPL), IL-1R7 | | |
| CD220 | Insulin R, Insulin receptor (INSR), IR | m IgG2b | 37.90 |
| CD221 | Insulin-like growth factor 1 receptor (IGF1R), | m IgG2a | 4.11 |
| | IGFR, type I IGF receptor (IGF-IR), JTK13 | | |
| CD222 | Cation-independent mannose-6-phosphate | m IgG2a | 37.39 |
| | receptor (M6P-R, CIM6PR, CIMPR, CIMPR), | | |
| | Insulin-like growth factor 2 receptor (IGF2R, | | |
| | IGFIIR, IGF-IIR), MPR1, MPRI | | |
| CD223 | Lymphocyte activation gene 3 (LAG3, LAG-3), | m IgG2a | 0.01 |
| | FDC protein | | |
| CD226 | DNAX accessory molecule 1 (DNAM-1), | m IgG2a | 83.32 |
| | Platelet and T-cell activation antigen 1 (PTA- | | |
| | 1), T lineage-specific activation antigen 1 | | |
| | antigen (TLiSA1) | | |
| CD227 | Mucin 1 (MUC1, MUC-1), DF3 antigen, H23 | m IgG2a | 20.90 |
| | antigen, Peanut-reactive urinary mucin | | |
| | (PUM), Polymorphic epithelial mucin (PEM), | | |
| | Epithelial membrane antigen (EMA), Tumor- | | |
| | associated mucin, Episialin | | |
| CD229 | Lymphocyte antigen 9 (Ly9), T-lymphocyte | m IgG2a | 0.00 |
| | surface antigen Ly-9, Signaling lymphocyte | | |
| | activation molecule family member 3 | | |
| | (SLAMF3), Lgp100, T100 | | |
| CD230 | Prion protein (PrP, PRNP), Major prion | m IgG2a | 98.25 |
| | protein, prP27-30, prP33-35C, PrPc | | |
| CD231 | A15, Tetraspanin 7 (TSPAN7), T-cell acute | m IgG1 | 5.20 |
| | lymphoblastic leukemia-associated antigen 1 | | |
| | (TALLA-1), Transmembrane 4 superfamily | | |
| | member 2 (TM4SF2), Membrane component | | |
| | X chromosome surface marker-1 (MXS1) | | |
| CD234 | Duffy, Duffy antigen/chemokine receptor | m IgG2a | 11.68 |
| | (DARC), Duffy blood group antigen (Dfy, FY), | | |
| | Fy-Glycoprotein, Glycoprotein D | | |
| CD235ab | Glycophorin A/B | m IgG2b | 96.93 |
| CD235a | Glycophorin A (GYPA), Sialoglycoprotein α, | m IgG2b | 0.01 |
| | Sialoglycoprotein A, MN blood group antigen, | | |
| | PAS-2 | | |
| CD238 | B-CAM, Kell blood group glycoprotein (Kel), | m IgG2a | 1.08 |
| | Kell blood group antigen, Endothelin-3- | | |
| | converting enzyme (ECE3), Kell | | |
| CD239 | Rh30CE, Basal cell adhesion molecule (BCAM, | m IgG2a | 2.47 |
| | B-CAM), Lutheran blood group glycoprotein, | | |
| | Lutheran blood group antigen (Lu) | | |
| CD243 | MDR-1, P-gp, GP170, p170, ABC-B1, ABC20, | m IgG2a | 43.84 |
| | CD243, CLCS, PGY1 | | |
| CD244 | 2B4, p38, NKLR2B4, NAIL, Nmrk, SLAMF4 | m IgG2a | 55.35 |
| CD247 | CD3-z, CD3H, CD3Q, CD3Z, T3Z, TCRZ, TCRz, | m IgG1 | 10.32 |
| | Zeta chain | | |
| CD252 | OX40L, OX-40L, TNFSF4, GP34, TXGP1, | m IgG2a | 53.27 |
| | CD134L | | |
| CD253 | TRAIL, TNFSF10, TL2, APO2L, Apo-2L | m IgG2a | 5.47 |
| CD254 | TRANCE, RANKL, TNFSF11, OPGL, ODF, sOdf, | m IgG2b | 10.04 |
| | OPTB2, hRANKL2 | | |
| CD255 | TWEAK, TNFSF12, APO3L | m IgG3 | 10.26 |
| CD257 | BAFF, BLyS, TNFSF13b, TALL1, THANK, | m IgG1 | 74.44 |
| | TNFSF20, ZTNF4 | | |
| CD258 | LIGHT, TNFSF14, LTg, TR2, HVEML | m IgG2a | 40.51 |
| CD261 | DR4, TRAIL-R1, TNFRSF10a, APO2, MGC9365 | m IgG2a | 25.10 |
| CD262 | DR5, TRAIL-R2, KILLER, TNFRSF10b, TRICK2, | m IgG2a | 21.38 |
| | TRICK2A, TRICK2B, TRICKB, ZTNFR9 | | |
| CD263 | DcR1, TRAIL-R3, TRID, TNFRSF10c, LIT | m IgG2a | 5.49 |
| CD264 | TRAIL-R4, DcR2, TNFSF10d, TRUNDD | m IgG2a | 1.81 |
| CD265 | TRANCE-R, RANK, TNFRSF11a, EOF, FEO, | m IgG2a | 3.27 |
| | ODFR, OFE, PDB2 | | |
| CD266 | TWEAK Receptor, TWEAK-R, TNFRSF12A, | m IgG2b | 4.12 |
| | FN14, FGFinducible 14 | | |
| CD267 | TACI, TNFRSF13B, CVID, FLJ39942, | m IgG2a | 3.77 |
| | MGC39952, MGC133214, TNFRSF14B | | |
| CD268 | BAFFR, BR3, TNFRSF13C, TR13C, CD268, | m IgG2a | 63.69 |
| | BAFF-R, MGC138235 | | |
| CD269 | BCMA, TNFRSF17, BCM | m IgG2a | 4.71 |
| CD270 | HVEM, TR2, HVEA, TNFRSF14, ATAR | m IgG2a | 99.80 |
| CD271 | NGFR (p75), p75NGFR, p75NTR, TNFRSF16, | m IgG1 | 30.04 |
| | Gp80-LNGFR | | |
| CD272 | BTLA, BTLA1, FLJ16065, MGC129743 | m IgG2a | 93.34 |
| CD273 | B7DC, PDL2, PD-L2, PDCD1L2, PDCD1LG2, | m IgG2a | 1.01 |
| | Btdc, CD273, MGC142238, MGC142240, | | |
| | bA574F11.2 | | |
| CD274 | B7H1, B7-H, PDL1, PD-L1, PDCD1LG1, | m IgG2a | 52.80 |
| | PDCD1L1, MGC142294, MGC142296, CD274 | | |
| CD275 | B7H2, B7-H2, ICOSL, B7RP1, B7h, GL50, | m IgG2a | 1.90 |
| | ICOSLG, CD275, LICOS, B7RP-1, ICOS-L, | | |
| | KIAA0653 | | |
| CD276 | B7RP-2, B7H3, B7-H3, 4Ig-B7-H3 | m IgG1 | 2.40 |
| CD277 | BT3.1, BTN3A1, BTF5, MGC141880 | m IgG2a | 99.90 |
| CD278 | ICOS, AILIM, CD278, MGC39850 | m IgG2a | 7.85 |
| CD279 | PD1, SLEB2, PDC1, CD279, hPD-1, PDCD1 | m IgG2a | 25.56 |
| CD281 | TLR1, TIL, rsc786, KIAA0012, MGC104956, | m IgG2a | 2.68 |
| | MGC126311, MGC126312, TIL.LPRS5, | | |
| | DKFZp547I0610, DKFZp564I0682 | | |
| CD282 | TLR2, TIL4, CD282 | m IgG2a | 2.45 |
| CD283 | TLR3, TOLL-like receptor 3 | m IgG2a | 75.40 |
| CD284 | TLR4, TOLL, hToll, ARMD10 | m IgG2a | 3.29 |
| CD286 | TLR6, TOLL-like receptor 6 | m IgG2a | 2.14 |
| CD289 | TLR9, TOLL-like receptor 9 | m IgG1 | 11.23 |
| CD290 | TLR10, TOLL-like receptor 10 | m IgG1 | 1.10 |
| CD292 | BMPR-IA, BMPR1A, ALK3, BIMPR1A, | m IgG2a | 5.39 |
| | 10q23del, ACVRLK3, SKR5 | | |
| CD294 | CRTH2, DP2, PGRD2, G protein-coupled | r IgG2a | 0.18 |
| | receptor 44 (GPR44), DL1R | | |
| CD295 | Leptin R, LEPR, OBR | m IgG2b | 4.86 |
| CD298 | ATP1B3, Na K ATPase β3 subunit, ATPB-3, | m IgG1 | 99.96 |
| | FLJ29027, ATP1β3 | | |
| CD299 | DC-SIGN/L, DC-SIGNR, L-SIGN, DCSIGN- | m IgG2a | 0.01 |
| | related, DCSIGNR, HP10347, DC-SIGN2, | | |
| | MGC47866, MGC12996, CLEC4M | | |
| CD300a | IRC1, IRC2, CLM-8, IRp60, IGSF12, CMRF35H, | m IgG1 | 15.32 |
| | CMRF-35H, CMRF35-H, CMRF-35-H9 | | |
| CD300c | CMRF35A, CMRF-35A, LIR, CLM-6, CMRF35, | m IgG2a | 0.18 |
| | IGSF16, CMRF-35, CMRF35A1, CMRF35-A1 | | |
| CD300e | CMRF35L1, CMRF-35L1, CLM2, CLM-2, IREM2, | m IgG2a | 0.11 |
| | PIgR2, IREM-2, PIgR-2, CD300LE, CMRF35-A5, | | |
| | CMRF35L | | |
| CD300f | IREM-1, IREM1, MAIR-V | m IgG2a | 0.00 |
| CD301 | CLEC10A, MGL1, CLECSF14, HML, MGL | m IgG2a | 4.93 |
| CD302 | CLEC13A, DCL1, BIMLEC | m IgG1 | 0.24 |
| CD303 | BDCA-2, BDCA2, CLEC4C, HECL | m IgG2a | 1.29 |
| CD304 | Neuropilin-1, BDCA-4, NRP1 | m IgG2a | 0.10 |
| CD305 | LAIR1 | m IgG1 | 83.79 |
| CD306 | LAIR2 | m IgG2b | 6.20 |
| CD307a | FcRH1, FCRL1, FCRH, IFGP1, IRTA5 | m IgG2a | 0.54 |
| CD307b | FCRL2, SPAP1, FcRH2, IFGP4, IRTA4 | m IgG1 | 3.85 |
| CD307c | FcRH3, FCRL3, IFGP3, IRTA3, SPAP2 | m IgG1 | 1.29 |
| CD307d | FCRL4, FCRH4, IGFP2, IRTA1 | m IgG2b | 1.60 |
| CD307e | FCRL5, BXMAS1, FCRH5, IRTA2, CD307 | m IgG2a | 0.03 |
| CD309 | VEGFR2, KDR, Flk1 | m IgG2a | 0.00 |
| CD312 | EMR2 | m IgG2b | 24.79 |
| CD314 | NKG2D, KLRK1 | m IgG1 | 72.35 |
| CD317 | BST2, PDCA-1, Tetherin, HM1.24 | m IgG1 | 68.85 |
| CD318 | CDCP1, SIMA135 | m IgG2b | 1.58 |
| CD319 | CRACC, CS1, SLAMF7 | m IgG2b | 47.43 |
| CD321 | JAM1, JAM, JAM-A, F11R | m IgG1 | 90.34 |
| CD324 | E-Cadherin, CDH1 | m IgG1 | 1.42 |
| CD325 | N-Cadherin, CDH2 CDw325, NCAD, CDH2 | m IgG1 | 1.03 |
| CD326 | Ep-CAM, MK-1, KSA, EGP40, TROP1, TACSTD1 | m IgG2a | 0.05 |
| CD328 | Siglec-7, p75/AIRM, Siglec7, AIRM-1 | m IgG1 | 9.18 |
| CD329 | Siglec-9 | m IgG2a | 9.18 |
| CD332 | FGFR2, BEK, K-SAM, KGFR | m IgG2a | 0.00 |
| CD333 | FGFR3, ACH, CEK2 | m IgG1 | 9.97 |
| CD334 | FGFR4, TKF, JTK2 | m IgG2a | 0.03 |
| CD335 | NKp46, NCR1, Ly94 | m IgG2a | 0.00 |
| CD336 | NKp44, NCR2, Ly-95 homolog, Ly95 | m IgG2b | 0.01 |
| CD337 | NKp30, NCR3, Ly-117 | m IgG2a | 0.13 |
| CD338 | ABCG2, ABCP, MXR, BCRP, Brcp1 | m IgG2b | 0.46 |
| CD339 | Jagged-1, JAG1, JAGL1, hJ1 | m IgG2b | 0.31 |
| CD340 | HER2/neu, Her-2, Neu, p185HER2, ERB-B2, | m IgG1 | 1.20 |
| | erbB2/HER-2 | | |
| CD344 | Frizzled-4, FZD4, EVR1, FEVR, Frizzled | m IgG2a | 18.24 |
| | homolog 4, Fz-4, hFz-4, FzE4 | | |
| CD352 | NTB-A, SLAMF6, Ly108 | m IgG1 | 99.86 |
| CD353 | SLAMF8, BLAME | m IgG2a | 0.70 |
| CD354 | TREM-1, TREM1 | m IgG1 | 1.57 |
| CD355 | CRTAM, Cytotoxic and regulatory T-cell | m IgG2a | 30.27 |
| | molecule | | |
| CD357 | TNFRSF18, Tumor necrosis factor receptor | m IgG1 | 1.33 |
| | superfamily, member 18, GITR, AITR | | |
| CD360 | IL-21R, IL21R | m IgG1 | 9.04 |
| CD362 | Syndecan-2 | r IgG2b | 0.08 |
| CD363 | S1PR1, Sphingosine-1-phosphate receptor 1, | m IgG2b | 1.30 |
| | EDG-1 | | |

Total 349
Positive 291

TABLE 5
Flow cytometric analysis of the multilineage
progenitor cells derived from CD19⁺ PBMCs which have been
cultured according to the method of the present invention
Proteins expression of CD19⁺ PBMCs by Flow Cytometry Analysis

| CD markers | Isotype | % of positive cells |
|---|---|---|
| αβTCR | m IgG3 | 12.28 |
| CLA | m IgG3 | 6.79 |
| EGFR | m IgG3 | 4.93 |
| HER-2 (c-new) | m IgG3 | 14.81 |
| HLA-A, B, C | m IgG3 | 99.64 |
| HLA-A2 | m IgG3 | 63.44 |
| HLA-DQ | m IgG3 | 65.68 |
| HLA-DR | m IgG3 | 98.69 |
| Integrin-β7 | m IgG3 | 19.63 |
| MIC A/B | m IgG3 | 0.26 |
| MHC Class I free chain without | m IgG3 | 0.01 |
| beta2 microglobulin | | |
| SSEA-1 | m IgG3 | 7.78 |
| SSEA-3 | m IgG3 | 11.58 |
| SSEA-4 | m IgG3 | 20.76 |
| TRA-1-60 | m IgG3 | 6.33 |
| TRA-1-81 | m IgG3 | 7.48 |
| Vβ8 | m IgG3 | 0.23 |
| Vβ23 | m IgG3 | 0.34 |

| | |
|---|---|
| Total | 18 |
| Positive | 14 |

TABLE 6
Flow cytometric analysis of the multilineage
progenitor cells derived from CD19⁺ PBMCs which have been
cultured according to the method of the present invention
CD markers expression fo CD19⁺ PBMCs by Flow Cytometry Analysis

| CD markers | Alternate names | Isotype | % of positive cells |
|---|---|---|---|
| CD1a | R4, T6, Leu-6, HTA1 | m IgG1 | 5.83 |
| CD1b | R1, T6 | m IgG1 | 0.01 |
| CD1c | BDCA-1, R7, T6, M241 | m IgG1 | 0.84 |
| CD1d | R3, R3G1 | m IgG2b | 37.13 |
| CD2 | T11, LFA-2, SRBC-R, E-rosette R, Erythrocyte R | m IgG1 | 31.65 |
| CD3 | T3 | m IgG1 | 25.28 |
| CD4 | T4, Leu-3, L3T4, Leu-3a, W3/25 | m IgG1 | 18.39 |
| CD5 | T1, Tp67, Leu-1, Ly-1 | m IgG1 | 47.05 |
| CD6 | T12, TP120 | m IgG1 | 66.66 |
| CD7 | gp40, Leu-9, TP41 | m IgG2a | 32.29 |
| CD8 | T8, Leu-2 | m IgG1 | 12.82 |
| CD8a | type I glycoprotein | m IgG1 | 14.76 |
| CD8b | Lyt3 | m IgG1 | 4.49 |
| CD9 | p24, MRP-1, DRAP-27, DRAP-1 | m IgG1 | 35.30 |
| CD10 | CALLA, NEP, gp100, EC 3.4.24.11, MME | m IgG2b | 3.43 |
| CD11a | LFA-1, integrin αL, ITGAL, LFA-1α | m IgG1 | 44.61 |
| CD11b | Mac-1, integrin αM, CR3, ITGAM, Mo1, C3niR | m IgG1 | 8.69 |
| CD11c | p150, 95, CR4, integrin αX, ITGAX, AXb2 | m IgG1 | 14.31 |
| CD13 | APN, gp150, Amniopeptidase N, ANPEP, AAP, | m IgG1 | 6.54 |
| | APM, LAP1, P150, PEPN, EC 3.4.11.2 | | |
| CD14 | LPS-Receptor | m IgG1 | 28.76 |
| CD15 | Lewis X, Lex, SSEA-1, 3-FAL, X-Hapten, FUT4 | m IgM | 8.45 |
| CD15s | Sialyl Lewis X | m IgM | 0.37 |
| CD16 | FCRIIIA, CD16a | m IgG1 | 6.29 |
| CD16b | FCRIIIB, FcγRIIIB | m IgG2a | 14.72 |
| CD17 | Lactosylceramide, LacCer | m IgG1 | 72.67 |
| CD18 | Integrin β2, ITGB2, CD11a, b, c β-subunit | m IgG1 | 94.74 |
| CD19 | B4 | m IgG1 | 65.77 |
| CD20 | B1, Bp35, Ly-44 | m IgG2b | 89.23 |
| CD21 | CR2, EBV-R, C3dR | m IgG2a | 76.16 |
| CD22 | BL-CAM, Siglec-2 | m IgG1 | 80.40 |
| CD23 | FcεRII, BLAST-2, FceRII, B6, Leu-20 | m IgG1 | 10.20 |
| CD24 | BA-1, HAS, HSA, BBA-1 | m IgG1 | 82.73 |
| CD25 | p55, IL-2Rα, Tac antigen, Tac, TCGFR | m IgG1 | 21.98 |
| CD26 | DPP IV ectoenzyme, DPP IV, ADA binding | m IgG1 | 17.81 |
| | protein, ADCP2, TP103 | | |
| CD27 | T14, S152, TNFRSF7, TP55 | m IgG1 | 43.22 |
| CD28 | Tp44, T44 | m IgG1 | 15.03 |
| CD29 | Integrin β1, platelet GPIIa, ITGB1, GP | m IgG1 | 99.62 |
| CD30 | Ki-1, Ber-H2, TNFRSF8 | m IgG1 | 1.88 |
| CD31 | PECAM-1, endocam, GPIIa, Platelet | m IgG1 | 84.15 |
| | endothelial cell adhesion molecule, PECA1 | | |
| CD32 | FcγRII | m IgG1 | 74.98 |
| CD33 | p67, Siglec-3, My9, gp67, Sialic acid-binding | m IgG1 | 2.39 |
| | Ig-like lectin 3, Myeloid cell surface antigen | | |
| | CD33 | | |
| CD34 | gp105-120, Mucosialin, My10, Hematopoietic | m IgG1 | 67.34 |
| | progenitor cell antigen 1 (HPCA1) | | |
| CD35 | CR1, C3b/C4b receptor, Immune adherence | m IgG1 | 78.09 |
| | receptor, Complement receptor 1 | | |
| CD36 | GPIV, OKM5 antigen, PASIV, Glycoprotein IIIb | m IgM | 63.42 |
| | (GpIIIb), Glycoprotein IV (GPIV), Fatty acid | | |
| | translocase (FAT), SCARB3, GP88, Platelet | | |
| | glycoprotein 4 | | |
| CD37 | gp 52-40, Tspan-26, Leukocyte antigen CD37, | m IgG1 | 24.01 |
| | Tetraspanin-26, TSPAN26 | | |
| CD38 | T10, ADP-ribosyl cyclase, Cyclic ADP-ribose | m IgG1 | 87.54 |
| | hydrolase 1 | | |
| CD39 | NTPDase-1, gp80, EC3.6.1.5, Ectonucleoside | m IgG1 | 81.57 |
| | triphosphate diphosphohydrolase 1 | | |
| | (ENTPD1), ATPdehydrogenase | | |
| CD40 | Bp50, TNFRSF5, MGC9013, Tumor necrosis | m IgG1 | 83.29 |
| | factor receptor superfamily member 5 | | |
| CD41 | ITGA2B, GPIIb, Integrin αIIb, Platelet | m IgG1 | 20.24 |
| | membrane glycoprotein IIb, Integrin α2b, | | |
| | Human Platelet Antigen-3 (HPA-3) | | |
| CD41a | Integrin alpha Iib, platelet GPIIb | m IgG1 | 19.63 |
| CD41b | fibrinogen receptor, gpIIb/IIIa, integrin alpha | mIgG3 | 51.23 |
| | IIb, ITGA2b | | |
| CD42a | GPIX, GP9, Platelet glycoprotein IX | m IgG1 | 22.08 |
| CD42b | gpIbα, GPIba, Platelet glycoprotein Ib α | m IgG1 | 29.28 |
| CD42d | Glycoprotein V, GPV, Platelet glycoprotein V | m IgG1 | 8.91 |
| CD43 | gpL115, Sialophorin, Leukosialin, | mIgG1 | 40.65 |
| | Galactoglycoprotein, SPN | | |
| CD44 | H-CAM, Pgp-1, EMCR III, CD44s, Hermes | m IgG2b | 95.61 |
| | antigen, ECMRII, Phagocytic glycoprotein I, | | |
| | Extracellular matrix receptor III, GP90 | | |
| | Lymphocyte homing/adhesion receptor, | | |
| | Hyaluronate receptor | | |
| CD45 | Leukocyte Common Antigen (LCA), T200, | m IgG1 | 99.82 |
| | B220, Ly5, Protein tyrosine phosphatase | | |
| | receptor type C (PTPRC) | | |
| CD45RA | PTPRC | m IgG2b | 91.55 |
| CD45RB | PTPRC | m IgG2b | 98.22 |
| CD45RO | UCHL-1 | m IgG2a | 13.75 |
| CD46 | Membrane Cofactor Protein (MCP), | m IgG1 | 99.78 |
| | Trophoblast leukocyte common antigen, | | |
| | TRA2.10 | | |
| CD47 | IAP, neurophilin, gp42, OA3, MER6 | m IgG1 | 99.65 |
| CD48 | Blast-1, BCM1, Sgp-60, SLAMF2, Hulym3, OX- | m IgG1 | 96.91 |
| | 45, MEM-102 | | |
| CD49a | VLA-1α, Integrin α1, VLA-1, ITGA1 | m IgG1 | 6.47 |
| CD49b | VLA-2α, gpIa, Integrin α2, VLA-2, ITGA2 | m IgG1 | 65.83 |
| CD49c | VLA-3α, Integrin α3, VLA-3, ITGA3, GAPB3, | m IgG1 | 2.47 |
| | Galactoprotein B3, MSK18, Very Common | | |
| | Antigen-2 (VCA-2) | | |
| CD49d | VLA-4α, Integrin α4, VLA-4, ITGA4 | m IgG1 | 97.64 |
| CD49e | VLA-5α, Integrin α5, VLA-5, ITGA5, | m IgG3 | 20.32 |
| | Fibronectin receptor | | |
| CD49f | VLA-6α, Integrin α6, VLA-6, ITGA6, gpI | r IgG2a | 20.73 |
| CD50 | ICAM-3 | m IgG1 | 99.71 |
| CD51/61 | vitronectin R, Integrin αv, VNR-α, Vitronectin- | m IgG1 | 25.27 |
| | Rα, ITGAV, Integrin αvβ3 | | |
| CD52 | CAMPATH-1, HE5, Epididymal secretory | m IgG2b | 97.79 |
| | protein E52, HES | | |
| CD53 | OX-44, MCR, TSPAN25, MOX44, Tetraspanin- | m IgG1 | 98.31 |
| | 25 | | |
| CD54 | ICAM-1 | m IgG1 | 76.78 |
| CD55 | Decay Accelerating Factor for Complement | m IgG1 | 99.43 |
| | (DAF) | | |
| CD56 | Leu-19, NKH-1, Neural Cell Adhesion | m IgG1 | 5.84 |
| | Molecule (NCAM) | | |
| CD57 | HNK-1, Leu-7, β-1,3-glucuronyltransferase 1, | m IgM | 22.21 |
| | Glucuronosyltransferase P, | | |
| | galactosylgalactosylxylosylprotein 3-β- | | |
| | glucuronosyltransferase 1 | | |
| CD58 | LFA-3 | m IgG1 | 0.04 |
| CD59 | Protectin, H19, 1F-5Ag, MIRL, MACIF, P-18 | m IgG1 | 98.69 |
| CD61 | GP IIIa, Integrin β3 | m IgG1 | 39.96 |
| CD62E | E-selectin, ELAM-1, LECAM-2 | m IgG1 | 0.99 |
| CD62L | L-selectin, LECAM-1, LAM-1, Leu-8, TQ1, MEL- | m IgG1 | 60.41 |
| | 14 | | |
| CD62P | P-selectin, GMP-140, PADGEM | m IgG1 | 4.68 |
| CD63 | LIMP, MLA1, LAMP-3, ME491, gp55, NGA, | m IgG1 | 74.65 |
| | OMA81H, TSPAN30, Granulophysin, | | |
| | Melanoma 1 antigen | | |
| CD64 | FcγRI, FcR I | m IgG1 | 1.53 |
| CD65 | Ceramide-dodecasaccharide, VIM2, | m IgM | 0.48 |
| | Fucoganglioside (Type II) | | |
| CD65s | Sialylated poly-N-acetyllactosamine, | m IgM | 28.97 |
| | Sialylated-CD65, VIM2 | | |
| CD66 | | m IgG2a | 28.93 |
| CD66abce | | m IgG2b | 0.98 |
| CD66a | NCA-160, BGP (Biliary glcoprotein), BGP1, | m IgG2a | 0.48 |
| | BGPI, CEACAM1 | | |
| CD66b | CD67, CGM6, NCA-95, CEACAM8 | m IgM | 0.34 |
| CD66c | NCA, NCA-50/90, CEAL, CEACAM6 | m IgG1 | 8.70 |
| CD68 | gp110, Macrosialin, SCARD1 | m IgG2b | 1.58 |
| CD69 | AIM, VEA, MLR3, EA 1, gp34/28, CLEC2C, BL- | m IgG1 | 1.35 |
| | AP26 | | |
| CD70 | Ki-24, CD27L, TNFSF7, CD27LG | m IgG1 | 15.32 |
| CD71 | TfR, T9, TFRC, Transferrin receptor, TRFR | m IgG1 | 23.86 |
| CD72 | Lyb-2, Ly-32.2, Ly-19.2 | m IgG2b | 29.00 |
| CD73 | NT5E, Ecto-5′-nuclotidase, E5NT, NT5, NTE, | m IgG1 | 45.27 |
| | eN, eNT | | |
| CD74 | Ii, invariant chain, DHLAG, HLADG, Ia-γ | m IgG1 | 38.59 |
| CD75 | lactosamines, ST6GAL1, MGC48859, SIAT1, | m IgM | 2.96 |
| | ST6GALL, ST6N, ST6 β-Galactosamide α-2,6- | | |
| | sialyltranferase, | | |
| | Sialo-masked lactosamine, Carbohydrate of | | |
| | α2,6 sialyltransferase | | |
| CD77 | Pk Ag, BLA, CTH, Gb3, Pk blood groupBLA, | m IgM | 36.61 |
| | A14GALT (α1,4-Galactosyltransferase), | | |
| | A4GALT1, Gb3S, P(k), P1, PK A4GALT, Pk | | |
| | antigen, CTH/Gb3A4GALT1, Gb3S, PK, P1 | | |
| CD79a | Igα, MB1, IGA (Immunoglobulin-associated a), | m IgG1 | 70.77 |
| | MB-1 | | |
| CD79b | B29, Igβ (Immunoglobulin-associated β) | m IgG1 | 86.14 |
| CD80 | B7, B7-1, BB1, CD28LG, CD28LG1, L AB7 | m IgG1 | 4.96 |
| CD81 | TAPA-1, S5.7 | m IgG1 | 2.66 |
| CD83 | HB15, BL11 | m IgG1 | 3.05 |
| CD84 | GR6, SLAMF5, LY9B, p75, hly9-β | m IgG1 | 0.00 |
| CD85a | ILT5, LIR3, HL9, LILRB3 (Leukocyte | r IgG2a | 3.15 |
| | immunoglobulin-like receptor, subfamily B | | |
| | (with TM and ITIM domains), member 3, LIR- | | |
| | 3, MGC138403, PIRB, XXbac-BCX105G6.7 | | |
| CD85d | ILT4, LILRB2 (Leukocyte immunoglobulin-like | r IgG2a | 11.54 |
| | receptor, subfamily B (with TM and ITIM | | |
| | domains), member 2, LIR2, MIR10, MIR-10 | | |
| CD85f | LIT11, LILRA5, XXbac-BCX403H19.2, LIR9, | m IgG2a | 30.85 |
| | LILRB7 (Leukocyte immunoglobulin-like | | |
| | receptor, subfamily B (with TM and ITIM | | |
| | domains), member 7 | | |
| CD85g | ILT7, LILRA4 (Leukocyte immunoglobulin-like | m IgG1 | 0.51 |
| | receptor, subfamily A (with TM domain), | | |
| | member 4, MGC129597, MGC129598, LIR4 | | |
| CD85h | ILT1, LILRA2 (Leukocyte immunoglobulin-like | r IgG2a | 1.35 |
| | receptor, subfamily A (with TM domain), | | |
| | member 2, LIR7, LIR-7, XXbac-BCX85G21.2, | | |
| | ILT-1 | | |
| CD85i | LILRA1 (Leukocyte immunoglobulin-like | m IgG2b | 0.12 |
| | receptor), subfamily A (with TM domain), | | |
| | member 1, LIR6, LIR-6, MGC126563 | | |
| CD85j | ILT2, LILRB1 (Leukocyte immunoglobulin-like | m IgG1 | 46.36 |
| | receptor, subfamily B (with TM and ITIM | | |
| | domains), member 1, FLJ37515, LIR-1, LIR1, | | |
| | MIR-7, MIR7 | | |
| CD85k | ILT3, LILRB4 (Leukocyte immunoglobulin-like | m IgG1 | 0.47 |
| | receptor, subfamily B (with TM and ITIM | | |
| | domains), member 4, LIR-5, HM18, LIR5, | | |
| | LILRB5 | | |
| CD86 | B70, B7-2, CD28LG2, LAB72, MGC34413 | m IgG2b | 7.62 |
| CD87 | UPA-R, PLAUR, URKR | m IgG1 | 0.00 |
| CD88 | C5aR, C5aR C5R1, C5R1, C5AR, C5A | m IgG2a | 2.60 |
| CD89 | FcaR, IgA R | m IgG1 | 3.31 |
| CD90 | Thy-1 | m IgG1 | 0.64 |
| CD91 | α2M-R, LRP, LRP1, α2MR, APOER, APR | m IgG1 | 7.30 |
| CD93 | C1qRp, C1QR1, C1qRP, MXRA4, C1qR(P), | m IgM | 0.51 |
| | Dj737e23.1, GR11 | | |
| CD97 | EMR1, BL-KDD/F12, TM&LN1 | m IgG1 | 0.02 |
| CD98 | 4F2, FRP-1, RL-388, SLC3A2, 4F2HC, 4T2HC, | m IgG1 | 33.69 |
| | MDU1, NACAE | | |
| CD99 | MIC2, E2, MIC2, MIC2X, MIC2Y, HBA71, | m IgG2a | 93.68 |
| | MSK5X | | |
| CD99R | E2, CD99 Mab restricted | m IgM | 63.73 |
| CD100 | SEMA4D, SEMAJ, coll-4, C9orf164, FLJ33485, | m IgM | 51.74 |
| | FLJ34282, FLJ39737, FLJ46484, M-sema-G, | | |
| | MGC169138, MGC169141, SEMAJ | | |
| CD101 | BB27, V7, P126, IGSF2, BA27, BPC#4, V7-LSB | m IgG1 | 0.06 |
| CD102 | ICAM-2, Ly60 | m IgG2a | 91.07 |
| CD103 | HML-1, Integrin αE, aIEL, ITGAE, OX62, HML1 | m IgG1 | 0.84 |
| CD104 | TSP-180, Integrin β4, TSP1180, ITGB4 | r IgG2b | 0.09 |
| CD105 | Endoglin, ENG, HHT1, ORW, SH-2 | m IgG1 | 26.48 |
| CD106 | VCAM-1, INCAM-110, V-CAM, INCAM-100 | m IgG1 | 0.41 |
| CD107a | LAMP-1, LAMPA, CD107a, LGP120 | m IgG1 | 33.76 |
| CD107b | LAMP-2, LAMPB | m IgG1 | 21.79 |
| CD108 | SEMA7A, JMH blood group antigen, JMH | m IgM | 37.91 |
| CD109 | 8A3, 7D1, E123, Platelet activation factor, | m IgG1 | 2.06 |
| | 8As, 150 kD TGF-β-1-binding protein, Platelet- | | |
| | specific Gov antigen | | |
| CD110 | MPL, TPO-R, C-MPL | m IgG2a | 33.27 |
| CD111 | PRR1, Nectin-1, PVRL1, HveC, HIgR, CLPED1, | m IgG1 | 0.88 |
| | Hve C1 | | |
| CD112 | PRR2, Nectin-2, HveB, PVRL2 | m IgG1 | 2.66 |
| CD114 | G-CSFR, CSF3R, HG-CSFR | m IgG1 | 0.02 |
| CD115 | CSF-1R, M-CSFR, c-fms, FMS, FIM2 | r IgG1 | 49.62 |
| CD116 | GM-CSFRα, GM-CSFRa, CDw116, CSF2R, | m IgG1 | 47.97 |
| | CSF2RAX, CSF2RAY, CSF2RX, CSF2RY, GM-CSF- | | |
| | R-α, GMCSFR, GMR, MGC3848, MGC4838 | | |
| CD117 | c-kit, SCFR, PBT | m IgG1 | 0.67 |
| CD118 | LIFR, gp190, SJS2, STWS, SWS | m IgG1 | 3.96 |
| CD119 | IFNγR, IFNγRα, CDw119, IFNGR1, IFNγRa | m IgG1 | 51.86 |
| CD120a | TNFR-I, p55, TNFRSF1A, CD120a, FPF, | m IgG1 | 11.40 |
| | MGC19588, TBP1, TNF-R, TNF-R55, TNFAR, | | |
| | TNFR1, TNFR55, TNFR60, p55-R, p60 | | |
| CD120b | TNFR-II, p80, TNFRSF1B, p75, TNFR p80 | r IgG2b | 25.40 |
| CD121a | IL-1R type I, IL-1RI, IL1R, CD121A, D2S1473, IL- | m IgG1 | 14.55 |
| | IR-α, IL1RA, P80 | | |
| CD121b | IL-1R type II, IL-1RII | m IgG1 | 7.04 |
| CD122 | IL-2Rβ, IL2RB, p70-75 | m IgG2a | 3.16 |
| CD123 | IL-3Rα, IL3RA, CD123, IL3R, IL3RAY, IL3RX, | m IgG1 | 11.83 |
| | IL3RY, MGC34174, hIL-3Ra | | |
| CD124 | IL-4Rα, IL4R | m IgG2a | 13.84 |
| CD125 | IL-5Rα, CDw125, IL5RA | m IgG1 | 36.72 |
| CD126 | IL-6Rα, IL6R | m IgG1 | 0.02 |
| CD127 | IL-7R, IL-7Rα, IL7R, p90 | m IgG1 | 10.66 |
| CD129 | IL-9R, IL-9Rα | m IgG2b | 43.92 |
| CD130 | gp130, IL-6Rβ, IL6ST, IL6ST, IL6-β | m IgG1 | 8.56 |
| CD131 | CSF2RB, IL3RB, IL5RB, CDw131, IL-3Rβ, | m IgG1 | 5.82 |
| | common β chain, IL-3R common β | | |
| CD132 | Common γ chain, IL-2Rγ, IL2RG | r IgG2a | 16.05 |
| CD133 | AC133, PROML1, Prominin 1, Hematopoietic | m IgG1 | 4.69 |
| | stem cell antigen, prominin-like 1 | | |
| CD134 | OX-40, TNFRSF4 | m IgG1 | 39.06 |
| CD135 | Flt3/Flk2, STK-1 | m IgG1 | 6.81 |
| CD136 | MSP-R, RON, p158-ron, CDw136, MST1R | m IgG1 | 65.39 |
| CD137 | 4-1BB, TNFRSF9, ILA | m IgG1 | 5.43 |
| CD137 | 4-1BB Ligand | m IgG1 | 39.43 |
| Ligand | | | |
| CD138 | Syndecan-1, Heparan sulfate proteoglycan | m IgG1 | 56.19 |
| CD140a | PDGFRA, PDGF α Receptor, PDGFRα | m IgG1 | 3.76 |
| CD140b | PDGFRB, PDGF β Receptor, PDGFRβ | m IgG1 | 22.94 |
| CD141 | Thrombomodulin, THBD, Fetomodulin | m IgG1 | 13.33 |
| CD142 | Tissue Factor (TF), Factor III, Thromboplastin | m IgG1 | 0.41 |
| CD144 | VE-Cadherin, Cadherin-5 | m IgG1 | 6.59 |
| CD146 | MUC18, S-endo, MCAM, Mel-CAM, Endo-CAM | m IgG1 | 0.40 |
| CD147 | Neurothelin, basigin, EMMPRIN, BSG, M6, | m IgG1 | 0.06 |
| | OX47, TCSF | | |
| CD148 | HPTP-eta, p260, DEP-1, HPTP-η, SCC1, PTPRJ | m IgG1 | 11.03 |
| CD150 | SLAM, IPO-3 | m IgG1 | 0.18 |
| CD151 | PETA-3, Tspan-24, RAPH, SFA-1 | m IgG1 | 0.00 |
| CD152 | CTLA-4 | m IgG2a | 0.04 |
| CD153 | CD30L, TNFSF8, TNSF8 | m IgG2b | 13.50 |
| CD154 | CD40L, T-BAM, gp39, TRAP, TNFSF5, TRAP-1, | m IgG1 | 8.13 |
| | IMD3 | | |
| CD155 | PVR, Necl-5, PVS, TAGE4, HVED, NECL5 | m IgG2a | 0.24 |
| CD156b | TACE, ADAM17, cSVP | m IgG1 | 16.65 |
| CD156c | ADAM10, MADM, kuz | m IgG2b | 97.99 |
| CD157 | BST-1, Bp3, Mo5 | m IgG1 | 0.17 |
| CD158a | KIR2DL1, p58.1, NKAT1 | m IgG2b | 1.64 |
| CD158b | p58.2, KIR2DL2/L3, NKAT2 | m IgG2a | 0.25 |
| CD158d | KIR2DL4, KIR103AS, KIR103 | m IgG1 | 0.73 |
| CD158e1 | KIR3DL1, NKB1, NKB1B, p70 | m IgG1 | 0.47 |
| CD158f | KIR2DL5A, KIR2DL5 | m IgG1 | 0.33 |
| CD159a | NKG2A, KLRC1 | m IgG2a | 0.20 |
| CD159c | NKG2C, KLRC2 | m IgG1 | 0.15 |
| CD160 | BY55, NK1, NK28 | m IgM | 1.43 |
| CD161 | NKR-P1A, KLRB1, NKR | m IgG1 | 0.54 |
| CD162 | PSGL-1 | m IgG2a | 21.57 |
| CD163 | M130, GHI/61, D11, RM3/1 | m IgG1 | 0.42 |
| CD164 | MGC-24, MUC-24, Endolyn | m IgG1 | 19.88 |
| CD165 | AD2, gp37 | m IgG1 | 44.68 |
| CD166 | ALCAM, KG-CAM, SC-1, BEN, DM-GRASP | m IgG1 | 1.63 |
| CD167a | DDR1, trkE, cak | m IgG3 | 26.65 |
| CD169 | Sialoadhesin, Siglec-1 | m IgG1 | 0.06 |
| CD170 | Siglec-5, CD33-like2 | m IgG1 | 14.75 |
| CD171 | L1CAM, N-CAM L1, L1 antigen, HSAS, HSAS1, | m IgG2a | 0.78 |
| | MASA, MIC5, S10, SPG1, NILE | | |
| CD172a | SIRP alpha, BIT, MFR, MYD-1, P84, SHPS-1, | m IgG2a | 0.00 |
| | SHPS1, SIRPα2, SIRPa | | |
| CD172b | SIRPβ, SIRPβ1 | m IgG1 | 0.02 |
| CD172g | SIRPγ, SIRPβ2, SIRPγ, SIRP-B2, bA77C3.1 | m IgG1 | 0.40 |
| CD177 | NB1, HNA-2a, NB1gp, Neutrophil-specific | m IgG1 | 0.11 |
| | antigen 1, PRV1 | | |
| CD178 | CD95L, TNFSF6, Fas Ligand, FasL, APT1LG1 | m IgG1 | 0.06 |
| CD179a | VpreB, IGVPB, VPREB1 | m IgG1 | 0.36 |
| CD179b | Igλ5, λ 5, 14.1, IGL5, IGGL1, IGO, lambda5 | m IgG1 | 13.78 |
| CD180 | RP105, LY64, Bgp95, Ly78 | m IgG1 | 75.87 |
| CD181 | CDw128A, IL-8RA, (formerly CD128a) CXCR1, | m IgG2b | 1.54 |
| | IL-8Rα | | |
| CD182 | CDw128B, IL-8RB, (formerly CD128b) CXCR2, | m IgG1 | 3.94 |
| | IL-8Rβ, CMKAR2, IL8R2 | | |
| CD183 | CXCR3, GPR9, CKR-L2, CMKAR3, IP10, Mig-R, TAC | m IgG1 | 32.23 |
| CD184 | CXCR4, Fusin, LESTR, NPY3R, CMKAR4, HM89, | m IgG2a | 97.30 |
| | FB22, LCR1 | | |
| CD185 | CXCR5, BLR1, MDR15, MGC117347 | m IgG2b | 90.11 |
| CD186 | CXCR6, CDw186, STRL33, TYMSTR, BONZO | m IgG2b | 5.67 |
| CD191 | CCR1, CKR1, CKR-1, HM145, CMKBR1, | m IgG2b | 0.02 |
| | SCYAR1, MIP-1αR, RANTES-R | | |
| CD192 | CCR2, CKR2, CCR2A, CCR2B, CKR2A, CKR2B, | m IgG2a | 4.42 |
| | CMKBR2, MCP-1-R, CC-CKR-2, FLJ78302, | | |
| | MGC103828, MGC111760, MGC168006 | | |
| CD193 | CCR3, CKR3, CMKBR3, CC-CKR-3, MGC102841 | m IgG2b | 0.56 |
| CD194 | CCR4, CC-CKR-4, CKR4, CMKBR4, ChemR13, | m IgG2b | 5.47 |
| | HGCN | | |
| CD195 | CCR5, CMKBR5, IDDM22, CC-CKR-5, FLJ78003 | m IgG1 | 0.50 |
| CD196 | CCR6, LARC receptor, DRY6, BN-1, DCR2, | m IgG1 | 2.82 |
| | CKRL3, GPR29, CKR-L3, CMKBR6, GPRCY4, | | |
| | STRL22, CC-CKR-6 | | |
| CD197 | EBI-1, BLR-2, CMKBR7, CCR7 (formerly | r IgG2a | 2.09 |
| | CDw197) | | |
| CDw198 | CCR8, CKR-L1, CKRL1, CMKBR8, CMKBRL2, | r IgG2b | 1.90 |
| | CY6, GPR-CY6, TER1 | | |
| CDw199 | CCR9, GPR28, GPR-9-6 | m IgG2a | 20.89 |
| CD200 | OX2, MRC, MOX1, MOX2 | m IgG1 | 28.10 |
| CD201 | EPC-R, PROCR, CCCA, CCD41, MGC23024, | r IgG1 | 0.00 |
| | bA42O4.2 | | |
| CD202b | Tie2 (Tek), TEK, VMCM, TIE-2, VMCM1 | m IgG1 | 0.14 |
| CD203c | E-NPP3, PD-1b, PDNP3, B10, PDIβ | m IgG1 | 0.16 |
| CD204 | MSR, MSR1, SR-A, phSR1, phSR2, SCARA1 | m IgG2b | 0.99 |
| CD205 | DEC-205, CLEC13B, GP200-MR6, LY75 | m IgG2b | 0.23 |
| CD206 | Mannose receptor C type-1 (MRC1), | m IgG1 | 3.56 |
| | Macrophage mannose receptor (MMR), C- | | |
| | type Lectin domain family 13 member D | | |
| | (CLEC13D) | | |
| CD207 | Langerin, C-type Lectin domain family 4 | m IgG1 | 0.49 |
| | member K (CLEC4K) | | |
| CD208 | DC-LAMP, Lysosomal-associated membrane | m IgG2a | 0.00 |
| | protein 3 (LAMP3), DCLAMP, LAMP, TSC403 | | |
| CD209 | Dendritic cell-specifi c ICAM-3-grabbing non- | r IgG2a | 1.63 |
| | integrin (DC-SIGN), DC-SIGN1, CDSIGN, C-type | | |
| | lectin domain family 4 member L (CLEC4L), | | |
| | HIV gp120-binding protein | | |
| CD210 | IL-10R | r IgG2a | 3.34 |
| CD210a | Interleukin 10 Receptor A (IL-10RA, IL-10R1), | m IgG1 | 14.60 |
| | IL-10Rα | | |
| CD210b | CRF2-4, Interleukin 10 Receptor B (IL-10RB, IL- | m IgG1 | 4.95 |
| | 10R2), IL-10Rβ, D21S58, CRFB4 | | |
| CD212 | IL-12Rβ1, IL12RB1, IL-12Rb1, Interleukin 12 | m IgG1 | 0.29 |
| | receptor β1 chain (IL-12β1), IL-12β, CD212b1 | | |
| CD213a1 | Interleukin 13 receptor α1 chain (IL-13Rα1), | m IgG2b | 0.15 |
| | NR4 | | |
| CD213a2 | IL12Rα2, IL-13Ra2, Interleukin 13 receptor α2 | m IgG1 | 2.30 |
| | chain (IL-13Rα2), interleukin-13-binding | | |
| | protein (IL13BP), IL13RA2 | | |
| CD215 | IL-15Rα, Interleukin 15 receptor alpha chain | m IgG2b | 20.35 |
| | (IL-15RA) | | |
| CD217 | IL-17R, CDw217, Interleukin 17 receptor A (IL- | m IgG1 | 0.18 |
| | 17RA) | | |
| CD218a | IL-18 Receptor alpha, IL18Rα, IL-1Rrp1, IL- | m IgG1 | 0.83 |
| | 18R, Interleukin 18 receptor 1 (IL-18R1), IL- | | |
| | 18RA, IL1 receptor-related protein (IL-1Rrp), | | |
| | IL-R5, CDw218a | | |
| CD218b | IL-1RcPL, CDw218b, Interleukin 18 receptor β | m IgG2b | 20.45 |
| | (IL-18Rβ), IL-18 receptor accessory protein (IL- | | |
| | 18RAP, IL-18RAcP), IL-1R accessory protein- | | |
| | like (IL-1RAcPL), IL-1R7 | | |
| CD220 | Insulin R, Insulin receptor (INSR), IR | m IgG2b | 0.34 |
| CD221 | Insulin-like growth factor 1 receptor (IGF1R), | m IgG1 | 0.33 |
| | IGFR, type I IGF receptor (IGF-IR), JTK13 | | |
| CD222 | Cation-independent mannose-6-phosphate | m IgG1 | 27.74 |
| | receptor (M6P-R, CIM6PR, CIMPR, CIMPR), | | |
| | Insulin-like growth factor 2 receptor (IGF2R, | | |
| | IGFIIR, IGF-IIR), MPR1, MPRI | | |
| CD223 | Lymphocyte activation gene 3 (LAG3, LAG-3), | m IgG1 | 0.04 |
| | FDC protein | | |
| CD226 | DNAX accessory molecule 1 (DNAM-1), | m IgG1 | 5.22 |
| | Platelet and T-cell activation antigen 1 (PTA- | | |
| | 1), T lineage-specifi c activation antigen 1 | | |
| | antigen (TLiSA1) | | |
| CD227 | Mucin 1 (MUC1, MUC-1), DF3 antigen, H23 | m IgG1 | 13.88 |
| | antigen, Peanut-reactive urinary mucin | | |
| | (PUM), Polymorphic epithelial mucin (PEM), | | |
| | Epithelial membrane antigen (EMA), Tumor- | | |
| | associated mucin, Episialin | | |
| CD229 | Lymphocyte antigen 9 (Ly9), T-lymphocyte | m IgG1 | 0.00 |
| | surface antigen Ly-9, Signaling lymphocyte | | |
| | activation molecule family member 3 | | |
| | (SLAMF3), Lgp100, T100 | | |
| CD230 | Prion protein (PrP, PRNP), Major prion | m IgG1 | 72.86 |
| | protein, prP27-30, prP33-35C, PrPc | | |
| CD231 | A15, Tetraspanin 7 (TSPAN7), T-cell acute | m IgG1 | 0.46 |
| | lymphoblastic leukemia-associated antigen 1 | | |
| | (TALLA-1), Transmembrane 4 superfamily | | |
| | member 2 (TM4SF2), Membrane component | | |
| | X chromosome surface marker-1 (MXS1) | | |
| CD234 | Duffy, Duffy antigen/chemokine receptor | m IgG2a | 6.81 |
| | (DARC), Duffy blood group antigen (Dfy, FY), | | |
| | Fy-Glycoprotein, Glycoprotein D | | |
| CD235ab | Glycophorin A/B | m IgG2b | 17.82 |
| CD235a | Glycophorin A (GYPA), Sialoglycoprotein α, | m IgG1 | 1.31 |
| | Sialoglycoprotein A, MN blood group antigen, | | |
| | PAS-2 | | |
| CD238 | B-CAM, Kell blood group glycoprotein (Kel), | m IgG1 | 0.09 |
| | Kell blood group antigen, Endothelin-3- | | |
| | converting enzyme (ECE3), Kell | | |
| CD239 | Rh30CE, Basal cell adhesion molecule (BCAM, | m IgG2a | 0.21 |
| | B-CAM), Lutheran blood group glycoprotein, | | |
| | Lutheran blood group antigen (Lu) | | |
| CD243 | MDR-1, P-gp, GP170, p170, ABC-B1, ABC20, | m IgG2a | 2.35 |
| | CD243, CLCS, PGY1 | | |
| CD244 | 2B4, p38, NKLR2B4, NAIL, Nmrk, SLAMF4 | m IgG1 | 0.36 |
| CD247 | CD3-z, CD3H, CD3Q, CD3Z, T3Z, TCRZ, TCRz, | m IgG1 | 1.69 |
| | Zeta chain | | |
| CD252 | OX40L, OX-40L, TNFSF4, GP34, TXGP1, | m IgG1 | 0.53 |
| | CD134L | | |
| CD253 | TRAIL, TNFSF10, TL2, APO2L, Apo-2L | m IgG1 | 0.51 |
| CD254 | TRANCE, RANKL, TNFSF11, OPGL, ODF, sOdf, | m IgG2b | 0.82 |
| | OPTB2, hRANKL2 | | |
| CD255 | TWEAK, TNFSF12, APO3L | m IgG3 | 3.04 |
| CD257 | BAFF, BLyS, TNFSF13b, TALL1, THANK, | m IgG1 | 20.08 |
| | TNFSF20, ZTNF4 | | |
| CD258 | LIGHT, TNFSF14, LTg, TR2, HVEML | m IgG1 | 0.18 |
| CD261 | DR4, TRAIL-R1, TNFRSF10a, APO2, MGC9365 | m IgG1 | 1.52 |
| CD262 | DR5, TRAIL-R2, KILLER, TNFRSF10b, TRICK2, | m IgG1 | 0.33 |
| | TRICK2A, TRICK2B, TRICKB, ZTNFR9 | | |
| CD263 | DcR1, TRAIL-R3, TRID, TNFRSF10c, LIT | m IgG1 | 0.31 |
| CD264 | TRAIL-R4, DcR2, TNFSF10d, TRUNDD | m IgG1 | 0.50 |
| CD265 | TRANCE-R, RANK, TNFRSF11a, EOF, FEO, | m IgG1 | 2.39 |
| | ODFR, OFE, PDB2 | | |
| CD266 | TWEAK Receptor, TWEAK-R, TNFRSF12A, | m IgG2b | 0.45 |
| | FN14, FGFinducible 14 | | |
| CD267 | TACI, TNFRSF13B, CVID, FLJ39942, | m IgG2a | 23.33 |
| | MGC39952, MGC133214, TNFRSF14B | | |
| CD268 | BAFFR, BR3, TNFRSF13C, TR13C, CD268, | m IgG2a | 86.50 |
| | BAFF-R, MGC138235 | | |
| CD269 | BCMA, TNFRSF17, BCM | m IgG2a | 0.97 |
| CD270 | HVEM, TR2, HVEA, TNFRSF14, ATAR | m IgG1 | 95.30 |
| CD271 | NGFR (p75), p75NGFR, p75NTR, TNFRSF16, | m IgG1 | 0.50 |
| | Gp80-LNGFR | | |
| CD272 | BTLA, BTLA1, FLJ16065, MGC129743 | m IgG2a | 76.80 |
| CD273 | B7DC, PDL2, PD-L2, PDCD1L2, PDCD1LG2, | m IgG1 | 1.06 |
| | Btdc, CD273, MGC142238, MGC142240, | | |
| | bA574F11.2 | | |
| CD274 | B7H1, B7-H, PDL1, PD-L1, PDCD1LG1, | m IgG1 | 0.33 |
| | PDCD1L1, MGC142294, MGC142296, CD274 | | |
| CD275 | B7H2, B7-H2, ICOSL, B7RP1, B7h, GL50, | m IgG1 | 3.04 |
| | ICOSLG, CD275, LICOS, B7RP-1, ICOS-L, | | |
| | KIAA0653 | | |
| CD276 | B7RP-2, B7H3, B7-H3, 4Ig-B7-H3 | m IgG1 | 0.35 |
| CD277 | BT3.1, BTN3A1, BTF5, MGC141880 | m IgG1 | 96.98 |
| CD278 | ICOS, AILIM, CD278, MGC39850 | m IgG1 | 0.51 |
| CD279 | PD1, SLEB2, PDC1, CD279, hPD-1, PDCD1 | m IgG1 | 0.83 |
| CD281 | TLR1, TIL, rsc786, KIAA0012, MGC104956, | m IgG1 | 1.07 |
| | MGC126311, MGC126312, TIL.LPRS5, | | |
| | DKFZp547I0610, DKFZp564I0682 | | |
| CD282 | TLR2, TIL4, CD282 | m IgG2a | 0.66 |
| CD283 | TLR3, TOLL-like receptor 3 | m IgG2a | 0.69 |
| CD284 | TLR4, TOLL, hToll, ARMD10 | m IgG2a | 1.11 |
| CD286 | TLR6, TOLL-like receptor 6 | m IgG1 | 0.57 |
| CD289 | TLR9, TOLL-like receptor 9 | m IgG1 | 3.65 |
| CD290 | TLR10, TOLL-like receptor 10 | m IgG1 | 1.62 |
| CD292 | BMPR-IA, BMPR1A, ALK3, BIMPR1A, | m IgG1 | 6.85 |
| | 10q23del, ACVRLK3, SKR5 | | |
| CD294 | CRTH2, DP2, PGRD2, G protein-coupled | r IgG2a | 0.57 |
| | receptor 44 (GPR44), DL1R | | |
| CD295 | Leptin R, LEPR, OBR | m IgG2b | 19.03 |
| CD298 | ATP1B3, Na K ATPase β3 subunit, ATPB-3, | m IgG1 | 99.68 |
| | FLJ29027, ATP1β3 | | |
| CD299 | DC-SIGN/L, DC-SIGNR, L-SIGN, DCSIGN- | m IgG2a | 0.00 |
| | related, DCSIGNR, HP10347, DC-SIGN2, | | |
| | MGC47866, MGC12996, CLEC4M | | |
| CD300a | IRC1, IRC2, CLM-8, IRp60, IGSF12, CMRF35H, | m IgG1 | 4.88 |
| | CMRF-35H, CMRF35-H, CMRF-35-H9 | | |
| CD300c | CMRF35A, CMRF-35A, LIR, CLM-6, CMRF35, | m IgG1 | 0.07 |
| | IGSF16, CMRF-35, CMRF35A1, CMRF35-A1 | | |
| CD300e | CMRF35L1, CMRF-35L1, CLM2, CLM-2, IREM2, | m IgG1 | 0.90 |
| | PIgR2, IREM-2, PIgR-2, CD300LE, CMRF35-A5, | | |
| | CMRF35L | | |
| CD300f | IREM-1, IREM1, MAIR-V | m IgG1 | 0.00 |
| CD301 | CLEC10A, MGL1, CLECSF14, HML, MGL | m IgG2a | 2.88 |
| CD302 | CLEC13A, DCL1, BIMLEC | m IgG1 | 0.32 |
| CD303 | BDCA-2, BDCA2, CLEC4C, HECL | m IgG2a | 0.59 |
| CD304 | Neuropilin-1, BDCA-4, NRP1 | m IgG2a | 0.49 |
| CD305 | LAIR1 | m IgG1 | 61.01 |
| CD306 | LAIR2 | m IgG2b | 6.98 |
| CD307a | FcRH1, FCRL1, FCRH, IFGP1, IRTA5 | m IgG1 | 14.71 |
| CD307b | FCRL2, SPAP1, FcRH2, IFGP4, IRTA4 | m IgG1 | 2.53 |
| CD307c | FcRH3, FCRL3, IFGP3, IRTA3, SPAP2 | m IgG1 | 0.98 |
| CD307d | FCRL4, FCRH4, IGFP2, IRTA1 | m IgG2b | 0.56 |
| CD307e | FCRL5, BXMAS1, FCRH5, IRTA2, CD307 | m IgG2a | 0.00 |
| CD309 | VEGFR2, KDR, Flk1 | m IgG1 | 0.00 |
| CD312 | EMR2 | m IgG2b | 0.58 |
| CD314 | NKG2D, KLRK1 | m IgG1 | 0.60 |
| CD317 | BST2, PDCA-1, Tetherin, HM1.24 | m IgG1 | 39.21 |
| CD318 | CDCP1, SIMA135 | m IgG2b | 0.68 |
| CD319 | CRACC, CS1, SLAMF7 | m IgG2b | 6.73 |
| CD321 | JAM1, JAM, JAM-A, F11R | m IgG1 | 0.54 |
| CD324 | E-Cadherin, CDH1 | m IgG1 | 1.70 |
| CD325 | N-Cadherin, CDH2 CDw325, NCAD, CDH2 | m IgG1 | 0.37 |
| CD326 | Ep-CAM, MK-1, KSA, EGP40, TROP1, TACSTD1 | m IgG1 | 0.29 |
| CD328 | Siglec-7, p75/AIRM, Siglec7, AIRM-1 | m IgG1 | 0.92 |
| CD329 | Siglec-9 | m IgG2a | 0.10 |
| CD332 | FGFR2, BEK, K-SAM, KGFR | m IgG1 | 0.00 |
| CD333 | FGFR3, ACH, CEK2 | m IgG1 | 0.69 |
| CD334 | FGFR4, TKF, JTK2 | m IgG1 | 0.08 |
| CD335 | NKp46, NCR1, Ly94 | m IgG1 | 0.01 |
| CD336 | NKp44, NCR2, Ly-95 homolog, Ly95 | m IgG2b | 0.00 |
| CD337 | NKp30, NCR3, Ly-117 | m IgG1 | 0.22 |
| CD338 | ABCG2, ABCP, MXR, BCRP, Brcp1 | m IgG2b | 0.45 |
| CD339 | Jagged-1, JAG1, JAGL1, hJ1 | m IgG2b | 1.40 |
| CD340 | HER2/neu, Her-2, Neu, p185HER2, ERB-B2, | m IgG1 | 0.19 |
| | erbB2/HER-2 | | |
| CD344 | Frizzled-4, FZD4, EVR1, FEVR, Frizzled | m IgG1 | 5.56 |
| | homolog 4, Fz-4, hFz-4, FzE4 | | |
| CD352 | NTB-A, SLAM F6, Ly108 | m IgG1 | 96.39 |
| CD353 | SLAMF8, BLAME | m IgG1 | 3.00 |
| CD354 | TREM-1, TREM1 | m IgG1 | 0.46 |
| CD355 | CRTAM, Cytotoxic and regulatory T-cell | m IgG2a | 0.41 |
| | molecule | | |
| CD357 | TNFRSF18, Tumor necrosis factor receptor | m IgG1 | 0.78 |
| | superfamily, member 18, GITR, AITR | | |
| CD360 | IL-21R, IL21R | m IgG3 | 33.69 |
| CD362 | Syndecan-2 | m IgG3 | 0.67 |
| CD363 | S1PR1, Sphingosine-1-phosphate receptor 1, | m IgG3 | 10.48 |
| | EDG-1 | | |

| | |
|---|---|
| Total | 344 |
| Positive | 224 |

TABLE 7
Flow cytometric analysis of the multilineage
progenitor cells derived from CD20⁺ PBMCs which have been
cultured according to the method of the present invention
Proteins expression of CD20⁺ PBMCs by Flow Cytometry Analysis

| CD markers | Isotype | % of positive cells |
|---|---|---|
| HLA-A, B, C | m IgG2a | 99.96 |

| | |
|---|---|
| Total | 1 |
| Positive | 1 |

TABLE 8
Flow cytometric analysis of the multilineage
progenitor cells derived from CD20⁺ PBMCs which have been
cultured according to the method of the present invention
CD markers expression of CD20⁺ PBMCs by Flow Cytometry Analysis

| CD markers | Alternate names | Isotype | % of positive cells |
|---|---|---|---|
| CD7 | gp40, Leu-9, TP41 | m IgG2a | 5.13 |
| CD10 | CALLA, NEP, gp100, EC 3.4.24.11, MME | m IgG2b | 11.09 |
| CD11b | Mac-1, integrin αM, CR3, ITGAM, Mo1, | m IgG1 | 35.72 |
| | C3niR | | |
| CD31 | PECAM-1, endocam, GPIIa, Platelet | m IgG1 | 99.51 |
| | endothelial cell adhesion molecule, PECA1 | | |
| CD34 | gp105-120, Mucosialin, My10, | m IgG1 | 2.45 |
| | Hematopoietic progenitor cell antigen 1 | | |
| | (HPCA1) | | |
| CD35 | CR1, C3b/C4b receptor, Immune adherence | m IgG2a | 74.68 |
| | receptor, Complement receptor 1 | | |
| CD37 | gp 52-40, Tspan-26, Leukocyte antigen | m IgG1 | 97.43 |
| | CD37, Tetraspanin-26, TSPAN26 | | |
| CD38 | T10, ADP-ribosyl cyclase, Cyclic ADP-ribose | m IgG1 | 97.30 |
| | hydrolase 1 | | |
| CD44 | H-CAM, Pgp-1, EMCR III, CD44s, Hermes | m IgG2b | 99.83 |
| | antigen, ECMRII, Phagocytic glycoprotein I, | | |
| | Extracellular matrix receptor III, GP90 | | |
| | Lymphocyte homing/adhesion receptor, | | |
| | Hyaluronate receptor | | |
| CD45 | Leukocyte Common Antigen (LCA), T200, | m IgG1 | 99.55 |
| | B220, Ly5, Protein tyrosine phosphatase | | |
| | receptor type C (PTPRC) | | |
| CD49d | VLA-4α, Integrin α4, VLA-4, ITGA4 | m IgG1 | 99.71 |
| CD50 | ICAM-3 | m IgG2a | 99.84 |
| CD53 | OX-44, MCR, TSPAN25, MOX44, | m IgG2a | 98.41 |
| | Tetraspanin-25 | | |
| CD55 | Decay Accelerating Factor for Complement | m IgG1 | 99.56 |
| | (DAF) | | |
| CD57 | HNK-1, Leu-7, β-1,3-glucuronyltransferase 1, | m IgM | 13.45 |
| | Glucuronosyltransferase P, | | |
| | galactosylgalactosylxylosyl protein 3-β- | | |
| | glucuronosyltransferase 1 | | |
| CD59 | Protectin, H19, 1F-5Ag, MIRL, MACIF, P-18 | m IgG1 | 97.65 |
| CD61 | GP IIIa, Integrin β3 | m IgG1 | 22.90 |
| CD62L | L-selectin, LECAM-1, LAM-1, Leu-8, TQ1, | m IgG1 | 98.46 |
| | MEL-14 | | |
| CD63 | LIMP, MLA1, LAMP-3, ME491, gp55, NGA, | m IgG1 | 90.70 |
| | OMA81H, TSPAN30, Granulophysin, | | |
| | Melanoma 1 antigen | | |
| CD64 | FcγRI, FcR I | m IgG1 | 0.72 |
| CD65s | Sialylated poly-N-acetyllactosamine, | m IgM | 29.97 |
| | Sialylated-CD65, VIM2 | | |
| CD66a | NCA-160, BGP (Biliary glcoprotein), BGP1, | m IgG2a | 15.95 |
| | BGPI, CEACAM1 | | |
| CD77 | Pk Ag, BLA, CTH, Gb3, Pk blood groupBLA, | m IgM | 61.12 |
| | A14GALT (α1,4-Galactosyltransferase), | | |
| | A4GALT1, Gb3S, P(k), P1, PK A4GALT, Pk | | |
| | antigen, CTH/Gb3A4GALT1, Gb3S, PK, P1 | | |
| CD84 | GR6, SLAMF5, LY9B, p75, hly9-β | m IgG1 | 21.56 |
| CD85a | ILT5, LIR3, HL9, LILRB3 (Leukocyte | r IgG2a | 0.93 |
| | immunoglobulin-like receptor, subfamily B | | |
| | (with TM and ITIM domains), member 3, | | |
| | LIR-3, MGC138403, PIRB, XXbac- | | |
| | BCX105G6.7 | | |
| CD89 | FcaR, IgA R | m IgG1 | 5.01 |
| CD90 | Thy-1 | m IgG1 | 0.07 |
| CD91 | α2M-R, LRP, LRP1, α2MR, APOER, APR | m IgG1 | 99.99 |
| CD92 | SLC44A1, CTL1, CHTL1, RP11-287A8.1, p70, | m IgG2b | 78.49 |
| | CDw92 | | |
| CD105 | Endoglin, ENG, HHT1, ORW, SH-2 | m IgG1 | 62.12 |
| CD107a | LAMP-1, LAMPA, CD107a, LGP120 | m IgG1 | 72.81 |
| CD112 | PRR2, Nectin-2, HveB, PVRL2 | m IgG1 | 23.06 |
| CD116 | GM-CSFRα, GM-CSFRa, CDw116, CSF2R, | m IgG1 | 2.31 |
| | CSF2RAX, CSF2RAY, CSF2RX, CSF2RY, GM- | | |
| | CSF-R-α, GMCSFR, GMR, MGC3848, | | |
| | MGC4838 | | |
| CD117 | c-kit, SCFR, PBT | m IgG1 | 1.04 |
| CD120a | TNFR-I, p55, TNFRSF1A, CD120a, FPF, | m IgG1 | 8.85 |
| | MGC19588, TBP1, TNF-R, TNF-R55, TNFAR, | | |
| | TNFR1, TNFR55, TNFR60, p55-R, p60 | | |
| CD123 | IL-3Rα, IL3RA, CD123, IL3R, IL3RAY, IL3RX, | m IgG1 | 18.02 |
| | IL3RY, MGC34174, hIL-3Ra | | |
| CD124 | IL-4Rα, IL4R | m IgG2a | 13.78 |
| CD127 | IL-7R, IL-7Rα, IL7R, p90 | m IgG1 | 4.97 |
| CD129 | IL-9R, IL-9Rα | m IgG2b | 12.91 |
| CD131 | CSF2RB, IL3RB, IL5RB, CDw131, IL-3Rβ, | m IgG1 | 6.45 |
| | common β chain, IL-3R common β | | |
| CD135 | Flt3/Flk2, STK-1 | m IgG2a | 2.27 |
| CD141 | Thrombomodulin, THBD, Fetomodulin | m IgG2a | 26.66 |
| CD144 | VE-Cadherin, Cadherin-5 | m IgG2a | 32.53 |
| CD150 | SLAM, IPO-3 | m IgG2a | 2.63 |
| CD153 | CD30L, TNFSF8, TNSF8 | m IgG2b | 39.50 |
| CD159c | NKG2C, KLRC2 | m IgG2a | 2.46 |
| CD164 | MGC-24, MUC-24, Endolyn | m IgG2a | 87.27 |
| CD170 | Siglec-5, CD33-like2 | m IgG2a | 53.52 |
| CD183 | CXCR3, GPR9, CKR-L2, CMKAR3, IP10, Mig-R, TAC | m IgG2a | 83.13 |
| CD192 | CCR2, CKR2, CCR2A, CCR2B, CKR2A, CKR2B, | m IgG2a | 43.42 |
| | CMKBR2, MCP-1-R, CC-CKR-2, FLJ78302, | | |
| | MGC103828, MGC111760, MGC168006 | | |
| CD199 | CCR9, GPR28, GPR-9-6 | m IgG2a | 61.03 |
| CD218b | IL-1RcPL, CDw218b, Interleukin 18 receptor | m IgG2b | 51.79 |
| | β (IL-18Rβ), IL-18 receptor accessory protein | | |
| | (IL-18RAP, IL-18RAcP), IL-1R accessory | | |
| | protein-like (IL-1RAcPL), IL-1R7 | | |
| CD227 | Mucin 1 (MUC1, MUC-1), DF3 antigen, H23 | m IgG2a | 38.12 |
| | antigen, Peanut-reactive urinary mucin | | |
| | (PUM), Polymorphic epithelial mucin (PEM), | | |
| | Epithelial membrane antigen (EMA), Tumor- | | |
| | associated mucin, Episialin | | |
| CD235ab | Glycophorin A/B | m IgG2b | 39.04 |
| CD238 | B-CAM, Kell blood group glycoprotein (Kel), | m IgG2a | 0.48 |
| | Kell blood group antigen, Endothelin-3- | | |
| | converting enzyme (ECE3), Kell | | |
| CD243 | MDR-1, P-gp, GP170, p170, ABC-B1, ABC20, | m IgG2a | 60.67 |
| | CD243, CLCS, PGY1 | | |
| CD257 | BAFF, BLyS, TNFSF13b, TALL1, THANK, | m IgG2a | 85.74 |
| | TNFSF20, ZTNF4 | | |
| CD264 | TRAIL-R4, DcR2, TNFSF10d, TRUNDD | m IgG2a | 1.24 |
| CD270 | HVEM, TR2, HVEA, TNFRSF14, ATAR | m IgG2a | 98.87 |
| CD281 | TLR1, TIL, rsc786, KIAA0012, MGC104956, | m IgG2a | 5.10 |
| | MGC126311, MGC126312, TIL.LPRS5, | | |
| | DKFZp547I0610, DKFZp564I0682 | | |
| CD286 | TLR6, TOLL-like receptor 6 | m IgG2a | 3.20 |
| CD318 | CDCP1, SIMA135 | m IgG2b | 0.57 |
| CD324 | E-Cadherin, CDH1 | m IgG2a | 11.74 |
| CD333 | FGFR3, ACH, CEK2 | m IgG2a | 10.55 |

| | |
|---|---|
| Total | 64 |
| Positive | 59 |

TABLE 9
Flow cytometric analysis of the multilineage
progenitor cells derived from CD25⁺ PBMCs which have been
cultured according to the method of the present invention
Proteins expression of CD25⁺ PBMCs by Flow Cytometry Analysis

| CD markers | Isotype | % of positive cells |
|---|---|---|
| αβTCR | m IgG3 | 64.64 |
| HLA-A, B, C | m IgG3 | 98.70 |

| | |
|---|---|
| Total | 2 |
| Positive | 2 |

TABLE 10
Flow cytometric analysis of the multilineage
progenitor cells derived from CD25⁺ PBMCs which have been
cultured according to the method of the present invention
CD markers expression of CD25⁺ PBMCs by Flow Cytometry Analysis

| CD markers | Alternate names | Isotype | % of positive cells |
|---|---|---|---|
| CD7 | gp40, Leu-9, TP41 | m IgG2a | 70.34 |
| CD9 | p24, MRP-1, DRAP-27, DRAP-1 | m IgG1 | 36.24 |
| CD11b | Mac-1, integrin αM, CR3, ITGAM, Mo1, | m IgG1 | 3.19 |
| | C3niR | | |
| CD11c | p150, 95, CR4, integrin αX, ITGAX, AXb2 | m IgG1 | 1.59 |
| CD18 | Integrin β2, ITGB2, CD11a, b, c β-subunit | m IgG1 | 99.46 |
| CD26 | DPP IV ectoenzyme, DPP IV, ADA binding | m IgG1 | 56.44 |
| | protein, ADCP2, TP103 | | |
| CD30 | Ki-1, Ber-H2, TNFRSF8 | m IgG1 | 9.90 |
| CD31 | PECAM-1, endocam, GPIIa, Platelet | m IgG1 | 100.00 |
| | endothelial cell adhesion molecule, PECA1 | | |
| CD34 | gp105-120, Mucosialin, My10, | m IgG1 | 0.38 |
| | Hematopoietic progenitor cell antigen 1 | | |
| | (HPCA1) | | |
| CD38 | T10, ADP-ribosyl cyclase, Cyclic ADP-ribose | m IgG1 | 71.72 |
| | hydrolase 1 | | |
| CD44 | H-CAM, Pgp-1, EMCR III, CD44s, Hermes | m IgG2b | 96.62 |
| | antigen, ECMRII, Phagocytic glycoprotein I, | | |
| | Extracellular matrix receptor III, GP90 | | |
| | Lymphocyte homing/adhesion receptor, | | |
| | Hyaluronate receptor | | |
| CD45 | Leukocyte Common Antigen (LCA), T200, | m IgG1 | 99.20 |
| | B220, Ly5, Protein tyrosine phosphatase | | |
| | receptor type C (PTPRC) | | |
| CD49a | VLA-1α, Integrin α1, VLA-1, ITGA1 | m IgG1 | 4.64 |
| CD49b | VLA-2α, gpIa, Integrin α2, VLA-2, ITGA2 | m IgG1 | 89.72 |
| CD49c | VLA-3α, Integrin α3, VLA-3, ITGA3, GAPB3, | m IgG1 | 54.36 |
| | Galactoprotein B3, MSK18, Very Common | | |
| | Antigen-2 (VCA-2) | | |
| CD49d | VLA-4α, Integrin α4, VLA-4, ITGA4 | m IgG1 | 83.76 |
| CD49e | VLA-5α, Integrin α5, VLA-5, ITGA5, | m IgG3 | 68.72 |
| | Fibronectin receptor | | |
| CD49f | VLA-6α, Integrin α6, VLA-6, ITGA6, gpI | r IgG2a | 25.08 |
| CD50 | ICAM-3 | m IgG1 | 99.46 |
| CD55 | Decay Accelerating Factor for Complement | m IgG1 | 99.66 |
| | (DAF) | | |
| CD56 | Leu-19, NKH-1, Neural Cell Adhesion | m IgG1 | 25.58 |
| | Molecule (NCAM) | | |
| CD59 | Protectin, H19, lF-5Ag, MIRL, MACIF, P-18 | m IgG1 | 98.94 |
| CD62L | L-selectin, LECAM-1, LAM-1, Leu-8, TQ1, | m IgG1 | 95.06 |
| | MEL-14 | | |
| CD63 | LIMP, MLA1, LAMP-3, ME491, gp55, NGA, | m IgG1 | 69.00 |
| | OMA81H, TSPAN30, Granulophysin, | | |
| | Melanoma 1 antigen | | |
| CD71 | TfR, T9, TFRC, Transferrin receptor, TRFR | m IgG1 | 25.00 |
| CD73 | NT5E, Ecto-5′-nuclotidase, E5NT, NT5, NTE, | m IgG1 | 16.36 |
| | eN, eNT | | |
| CD84 | GR6, SLAMF5, LY9B, p75, hly9-β | m IgG1 | 51.12 |
| CD90 | Thy-1 | m IgG1 | 0.34 |
| CD95 | Fas, APO-1, TNFRSF6, CD178, FASLG, CD95L, | m IgG1 | 84.06 |
| | APT1LG1, APT1, FAS1, FASTM, ALPS1A, | | |
| | TNFSF6, FASL | | |
| CD99 | MIC2, E2, MIC2, MIC2X, MIC2Y, HBA71, | m IgG2a | 98.71 |
| | MSK5X | | |
| CD100 | SEMA4D, SEMAJ, coll-4, C9orf164, FLJ33485, | m IgM | 98.31 |
| | FLJ34282, FLJ39737, FLJ46484, M-sema-G, | | |
| | MGC169138, MGC169141, SEMAJ | | |
| CD102 | ICAM-2, Ly60 | m IgG2a | 96.69 |
| CD105 | Endoglin, ENG, HHT1, ORW, SH-2 | m IgG1 | 95.96 |
| CD108 | SEMA7A, JMH blood group antigen, JMH | m IgM | 75.65 |
| CD116 | GM-CSFRα, GM-CSFRa, CDw116, CSF2R, | m IgG1 | 2.48 |
| | CSF2RAX, CSF2RAY, CSF2RX, CSF2RY, GM- | | |
| | CSF-R-α, GMCSFR, GMR, MGC3848, | | |
| | MGC4838 | | |
| CD117 | c-kit, SCFR, PBT | m IgG1 | 2.19 |
| CD123 | IL-3Rα, IL3RA, CD123, IL3R, IL3RAY, IL3RX, | m IgG1 | 2.28 |
| | IL3RY, MGC34174, hIL-3Ra | | |
| CD124 | IL-4Rα, IL4R | m IgG2a | 3.70 |
| CD125 | IL-5Rα, CDw125, IL5RA | m IgG1 | 5.55 |
| CD127 | IL-7R, IL-7Rα, IL7R, p90 | m IgG1 | 100.00 |
| CD134 | OX-40, TNFRSF4 | m IgG1 | 4.29 |
| CD135 | Flt3/Flk2, STK-1 | m IgG1 | 100.00 |
| CD138 | Syndecan-1, Heparan sulfate proteoglycan | m IgG1 | 3.13 |
| CD140b | PDGFRB, PDGF β Receptor, PDGFRβ | m IgG1 | 3.11 |
| CD144 | VE-Cadherin, Cadherin-5 | m IgG1 | 24.54 |
| CD148 | HPTP-eta, p260, DEP-1, HPTP-η, SCC1, PTPRJ | m IgG1 | 3.78 |
| CD150 | SLAM, IPO-3 | m IgG1 | 7.42 |
| CD153 | CD30L, TNFSF8, TNSF8 | m IgG2b | 11.85 |
| CD156c | ADAM10, MADM, kuz | m IgG2b | 99.84 |
| CD162 | PSGL-1 | m IgG2a | 94.58 |
| CD164 | MGC-24, MUC-24, Endolyn | m IgG1 | 97.76 |
| CD167a | DDR1, trkE, cak | m IgG3 | 75.47 |
| CD172g | SIRPγ, SIRPβ2, SIRPγ, SIRP-B2, bA77C3.1 | m IgG1 | 100.00 |
| CD183 | CXCR3, GPR9, CKR-L2, CMKAR3, IP10, Mig-R, TAC | m IgG1 | 99.96 |
| CD184 | CXCR4, Fusin, LESTR, NPY3R, CMKAR4, | m IgG2a | 95.90 |
| | HM89, FB22, LCR1 | | |
| CD223 | Lymphocyte activation gene 3 (LAG3, LAG- | m IgG1 | 5.35 |
| | 3), FDC protein | | |
| CD226 | DNAX accessory molecule 1 (DNAM-1), | m IgG1 | 57.33 |
| | Platelet and T-cell activation antigen 1 (PTA- | | |
| | 1), T lineage-specifi c activation antigen 1 | | |
| | antigen (TLiSA1) | | |
| CD230 | Prion protein (PrP, PRNP), Major prion | m IgG1 | 100.00 |
| | protein, prP27-30, prP33-35C, PrPc | | |
| CD235ab | Glycophorin A/B | m IgG2b | 16.62 |
| CD247 | CD3-z, CD3H, CD3Q, CD3Z, T3Z, TCRZ, TCRz, | m IgG1 | 100.00 |
| | Zeta chain | | |
| CD257 | BAFF, BLyS, TNFSF13b, TALL1, THANK, | m IgG1 | 9.62 |
| | TNFSF20, ZTNF4 | | |
| CD268 | BAFFR, BR3, TNFRSF13C, TR13C, CD268, | m IgG2a | 34.56 |
| | BAFF-R, MGC138235 | | |
| CD270 | HVEM, TR2, HVEA, TNFRSF14, ATAR | m IgG1 | 97.81 |
| CD272 | BTLA, BTLA1, FLJ16065, MGC129743 | m IgG2a | 54.26 |
| CD274 | B7H1, B7-H, PDL1, PD-L1, PDCD1LG1, | m IgG1 | 100.00 |
| | PDCD1L1, MGC142294, MGC142296, CD274 | | |
| CD277 | BT3.1, BTN3A1, BTF5, MGC141880 | m IgG1 | 96.99 |
| CD278 | ICOS, AILIM, CD278, MGC39850 | m IgG1 | 100.00 |
| CD298 | ATP1B3, Na K ATPase β3 subunit, ATPB-3, | m IgG1 | 99.37 |
| | FLJ29027, ATP1β3 | | |
| CD305 | LAIR1 | m IgG1 | 74.46 |
| CD317 | BST2, PDCA-1, Tetherin, HM1.24 | m IgG1 | 20.00 |
| CD318 | CDCP1, SIMA135 | m IgG2b | 2.44 |
| CD321 | JAM1, JAM, JAM-A, F11R | m IgG1 | 41.04 |

| | |
|---|---|
| Total | 72 |
| Positive | 70 |

TABLE 11
CD4+-PBMC shown various proteins expression by western blot analysis. A-F shown
proteins expression of CD4⁺-PBMC via semi-quantitative analysis of the percentage increase
in expression was determined by internal control beta-actin normalization. Five levels were
respectively exhibited by 0-20%, 21-40%, 41-60%, 61-80%, and 81-100%, represented as
“+”, “++”, “+++”, “++++”, and “+++++”.
A. Actin, Perforin, HLA Class I ABC, TAZ, Collagen I, ALP, and IGFBP3 of CD4⁺-PBMC
proteins expression

| | Proteins | | | | | | |
|---|---|---|---|---|---|---|---|
| % | | | HLA Class | | | | |
| Cells | Actin | Perforin | 1 ABC | TAZ | Collagen I | ALP | IGFBP3 |
| CD4⁺-PBMC | +++++ | +++++ | +++++ | +++++ | +++++ | +++++ | +++ |

B. Actin, PAX5, Fibronectin, TNFSF18, FLT-1, HIF-1 alpha, and WASP of CD4⁺-PBMC
proteins expression

| % | Proteins | | | | | | |
|---|---|---|---|---|---|---|---|
| Cells | Actin | PAX5 | Fibronectin | TNFSF18 | FLT-1 | HIF-1 alpha | WASP |
| CD4⁺-PBMC | +++++ | ++++ | ++++ | +++ | +++ | +++++ | +++++ |

C. Actin, CDX2, Annexin VI, GAD2, CAMK4, alpha-Actinin and Neuropilin-2 of CD4⁺-PBMC
proteins expression

| % | Proteins | | | | | | |
|---|---|---|---|---|---|---|---|
| Cells | Actin | CDX2 | Annexin VI | GAD2 | CAMK4 | α-Actinin | Neuropilin-2 |
| CD4⁺-PBMC | +++++ | +++++ | +++++ | +++++ | +++++ | ++++ | +++++ |

D. Actin, M-Cadherin, Sca-1, Notch 1, P-gp, NFYA, and MyoD1 of CD4⁺-PBMC proteins
expression

| | Proteins | | | | | | |
|---|---|---|---|---|---|---|---|
| % | | M- | | | | | |
| Cells | Actin | Cadherin | Sca-1 | Notch1 | P-gp | NFYA | MyoD1 |
| CD4⁺-PBMC | +++++ | +++++ | +++++ | ++++ | +++ | +++ | +++++ |

E. Actin, MAC-1, PU.1, Granulysin, Runx3, ASCL1, and Myogenin of CD4⁺-PBMC proteins
expression

| % | Proteins | | | | | | |
|---|---|---|---|---|---|---|---|
| Cells | Actin | MAC-1 | PU.1 | Granulysin | Runx3 | ASCL1 | Myogenin |
| CD4⁺-PBMC | +++++ | ++++ | ++ | ++ | ++ | + | ++ |

F. Actin, CRABP2, MRP1, Nestin, GATA4, G-CSF, and Caveolin-2 of CD4⁺-PBMC proteins
expression

| % | Proteins | | | | | | |
|---|---|---|---|---|---|---|---|
| Cells | Actin | CRABP2 | MRP1 | Nestin | GATA-4 | G-CSF | Caveolin-2 |
| CD4+-PBMC | +++++ | ++++ | +++++ | ++++ | +++++ | ++++ | ++++ |

G. Actin, Synaptophysin, Neurogenin 3, β Enolase, Granzyme B and NGF of CD4⁺-PBMC
proteins expression

| % | Proteins | | | | | |
|---|---|---|---|---|---|---|
| Cells | Actin | Synaptophysin | Neurogenin 3 | β Enolase | Granzyme B | NGF |
| CD4⁺-PBMC | +++++ | +++ | +++++ | ++++ | +++ | ++ |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Arthritis Res., 2, 477-488, 2000
Blood, 98, 2396-2402, 2001
Br. J. Haematol., 109, 235-242, 2000
C. Clare Blackburn & Nancy R. Manley “Developing a new paradigm for thymus organogenesis” Nature Reviews Immunology April 2004 278-289-Retrieved Oct. 4, 2012 [3]
J. Hepatol., 29, 676-682, 1998
Kim S. S. and Vacanti J. P., 1999. Semin Pediatr Surg. 8:119
Science, 284, 143-147, 1999
Science, 287, 1433-1438, 2000
Science, 287, 1442-1446, 2000
Sleckman B P, Lymphocyte antigen receptor gene assembly: multiple layers of regulation. Immunol Res 32:153-8, 2005 (full text (http://journals.humanapress.com/index.php?
U.S. Pat. No. 6,387,369 to Osiris, Therapeutics, Inc.
U.S. Pat. App. No. US20020094573A1 to Bell E

1.-27. (canceled)

28. A method of generating mammalian multilineage potential cells, comprising establishing an in vitro cell culture which proportionally comprises:
(a) 10-40% v/v of a mononuclear cell suspension which comprises one or more mononuclear cells that express CD4, CD8, CD25, CD20 or CD19;
(b) 5-40% v/v of an albumin solution; and
(c) 30-80% v/v of a cell culture medium,
wherein said in vitro cell culture is maintained for a time and under conditions sufficient to induce the transition of one or more of said mononuclear cells to one or more cells exhibiting multilineage differentiative potential.

29. The method of claim 28 in which (i) said mononuclear cell suspension is 20%-40% v/v of the in vitro cell culture, or (ii) said mononuclear cell suspension is 15% v/v of the in vitro cell culture.

30. The method of claim 28 wherein said mononuclear cell is a lymphocyte.

31. The method of claim 28 wherein said one or more mononuclear cells that express CD4 or said one or more mononuclear cells that express CD8 is a thymocyte, a T cell, a natural killer cell, a natural killer T cell, a macrophage or a dendritic cell.

32. The method of claim 31 wherein:
(a) said mononuclear cell suspension that comprises CD4⁺ or CD8⁺ mononuclear cells is present in the in vitro cell culture at 30% v/v,
(b) said albumin solution is present in the in vitro cell culture at 40% v/v, and
(c) said culture medium is present in the in vitro cell culture at 30% v/v.

33. The method of claim 28 wherein said mononuclear cell that expresses CD25 is a CD25⁺ regulatory T cell or a CD25⁺ memory T cell.

34. The method of claim 33 wherein:
(a) said mononuclear cell suspension that comprises CD25⁺ mononuclear cells is present in the in vitro cell culture at 20% v/v,
(b) said albumin solution is present in the in vitro cell culture at 40% v/v, and
(c) said culture medium is present in the in vitro cell culture at 40% v/v.

35. The method of claim 28 wherein said mononuclear cell that expresses CD19 or said mononuclear cell that expresses CD20 is a B cell at any stage of differentiation.

36. The method of claim 35 wherein:
(a) said mononuclear cell suspension that comprises CD19⁺ or CD20⁺ mononuclear cells is present in the in vitro cell culture at 40% v/v,
(b) said albumin solution is present in the in vitro cell culture at 20% v/v, and
(c) said culture medium is present in the in vitro cell culture at 40% v/v.

37. The method of claim 31 wherein said thymocyte is a double positive CD4⁺/CD8⁺ thymocyte.

38. The method of claim 30 wherein:
(i) the lymphocyte is a single positive CD4⁺ or CD8⁺ T cell or a CD8⁺ NK cell;
(ii) the lymphocyte is a CD25⁺ T regulatory cell;
(iii) the lymphocyte is a CD19⁺ B cell; or
(iv) the lymphocyte is a CD20⁺ B cell.

39. The method of claim 28 wherein said mononuclear cells are derived from peripheral blood or spleen.

40. The method of claim 28 wherein said cell exhibiting multilineage differentiative potential cell exhibits haematopoietic potentiality or mesenchymal potentiality.

41. The method of claim 28 wherein:
(i) said cell exhibiting multilineage differentiative potential cell is derived from a CD4⁺ mononuclear cell and expresses CD44 and CD45;
(ii) said cell exhibiting multilineage differentiative potential cell is derived from a CD8⁺ mononuclear cell and expresses CD45 and CD47;
(iii) said cell exhibiting multilineage differentiative potential cell is derived from a CD25⁺ mononuclear cell and expresses CD23; or
(iv) said cell exhibiting multilineage differentiative potential cell is derived from a CD19⁺ mononuclear cell and expresses CD44 and CD45.

42. The method of claim 40 wherein:
(i) haematopoietic potentiality is a potential to differentiate into a lymphocyte, monocyte, neutrophil, basophil, eosinophil, red blood cell or platelet; and
(ii) mesenchymal potentiality is a potential to differentiate into a bone, cartilage, smooth muscle, tendon, ligament, stroma, marrow, dermis or fat cell.

43. The method of claim 28 wherein said in vitro cell culture further comprises 10 mg/L insulin.

44. The method of claim 28 in which:
(i) the cell culture is maintained for 4-7 days; or
(ii) the cell culture is maintained for 4-5 days; or
(iii) the cell culture is maintained for 3-6 days.

45. The method of claim 28 wherein said mononuclear cells are human mononuclear cells.

46. The method of claim 28 which further comprises a step of contacting the cell exhibiting multilineage differentative potential (MLPC) with a stimulus to direct differentiation of said MLPC to a MLPC-derived phenotype.

47. The method of claim 46 wherein said MLPC-derived phenotype is a haematopoietic or mesenchymal phenotype.

48. The method of claim 47 wherein at least one of:
(i) the cell that has been directed to differentiate to a haemaopoietic phenotype has differentiated into a red blood cell, platelet, lymphocyte, monocyte, neutrophil, basophil or eosinophil;
(ii) the cell that has been directed to differentiate to a mesenchymal phenotype has differentiated into a bone, cartilage, smooth muscle, tendon, ligament, stroma, marrow, dermis or fat cell.

49. A method of therapeutically and/or prophylactically treating a condition in a mammal, comprising administering to said mammal:
(i) an effective number of cells exhibiting multilineage differentiative potential (MLPCs) that have been generated by the method of claim 28, or
(ii) an effective number of cells that have been partially or fully differentiated from MLPCs that have been generated by the method of claim 28.

50. The method of claim 49 wherein the condition is characterized by aberrant haematopoietic or mesenchymal function in the mammal.

51. The method of claim 50 wherein said condition is selected from a haematopoietic disorder, circulatory disorder, stroke, myocardial infarction, hypertension bone disorder, type II diabetes, infertility, damaged or morphologically abnormal cartilage or other tissue, hernia repair, pelvic floor prolapse surgery using supportive mesh and biological scaffolds, cell therapy for other musculoskeletal disorders, and replacement of defective supportive tissues in a context of aging, surgery or trauma.

52. A population of cells that is selected from (i) cells exhibiting multilineage differentiative potential (MLPCs) generated by the method of claim 28, or (ii) MLPC-derived cells obtained from the cells of (i).

53. A method of assessing an effect of a treatment or culture regime on a phenotypic or functional state of a cell exhibiting multilineage differentiative potential (MLPC) or a MLPC-derived cell, comprising:
(a) treating, by subjecting to said treatment or culture regime, a MLPC generated by the method of claim 28 or a MLPC-derived cell obtained therefrom, to obtain a treated MLPC or MLPC-derived cell; and
(b) screening the treated MLPC or MLPC-derived cell for an altered functional or phenotypic state, relative to the functional or phenotypic state of the MLPC or MLPC-derived cell prior to the step of treating, and therefrom assessing the effect of the treatment or culture regime.
